(19) United States
(12) Patent Application Publication
(10) Pub. No.: US 2009/0018150 A1
(43) Pub. Date: Jan. 15, 2009

(54) 5-Ht2b Receptor Antagonists

(75) Inventors: Richard Anthony Borman, Sawston (GB); Robert Alexander Coleman, Royston (GB); Kenneth Lyle Clark, Linton (GB); Alexander William Oxford, Royston (GB); George Hynd, Harlow (GB); Janet Ann Archer, Harlow (GB); Amanda Aley, Harlow (GB); Neil Victor Harris, Harlow (GB)

Correspondence Address: NIXON & VANDERHYE, PC, 901 NORTH GLEBE ROAD, 11TH FLOOR, ARLINGTON, VA, 22203, US

(73) Assignee: ASTERAND UK LIMITED, Royston, Hertfordshire (GB)

(21) Appl. No.: 10/564,010

(22) PCT Filed: Jul. 23, 2004
(86) PCT No.: PCT/GB2004/003184

§ 371 Date: Jan. 11, 2006

Related U.S. Application Data
(60) Provisional application No. 60/490,286, filed on Jul. 28, 2003.

(30) Foreign Application Priority Data
Jul. 24, 2003 (GB) 0317346.5

(51) Int. Cl. A61K 31/505; C07D 239/42; C07D 233/88; A61K 31/4168; A61K 31/421; C07D 263/48; C07D 249/14; A61K 31/4196; A61P 1/00

(52) U.S. Cl. 514/275; 544/297; 548/331.5; 514/398; 514/377; 548/233; 548/264.8; 514/383

(57) ABSTRACT

Compounds of formulae: (I), (II), (IIIa), (IIIb), (IVa) and (IVb): or a pharmaceutically acceptable salt thereof, for use as pharmaceuticals, in particular for the treatment of a condition alleviated by antagonism of a 5-HT_{2B} receptor.

This invention relates to 5-HT_{2B} receptor antagonists, pharmaceutical compositions comprising such compounds, and the use of such compounds and compositions to treat various diseases.

### BACKGROUND TO THE INVENTION

Serotonin, also referred to as 5-hydroxytryptamine (5-HT), is a neurotransmitter with mixed and complex pharmacological characteristics. 5-HT acts via a number of discrete 5-HT receptors. Currently, fourteen subtypes of serotonin receptor are recognised and delineated into seven families, 5-HT₁ to 5-HT₇. Within the 5-HT₂ family, 5-HT_{2A}, 5-HT_{2B} and 5-HT_{2C} subtypes are known to exist. The nomenclature and classification of 5-HT receptors has been reviewed by Martin and Humphrey, Neuropharm., 33, 261-273 (1994) and Hoyer, et al., Pharm. Rev., 46, 157-203 (1994).

There is evidence to suggest a role for 5-HT_{2B} receptors in a number of medical disorders, and therefore 5-HT_{2B} receptor antagonists are likely to have a beneficial effect on patients suffering these disorders. They include, but are not limited to: disorders of the GI tract, and especially disorders involving altered motility, and particularly irritable bowel syndrome (WO 01/08668); disorders of gastric motility, dyspepsia, GERD, tachygastria; migraine/neurogenic pain (WO 97/44326); pain (U.S. Pat. No. 5,958,934); anxiety (WO 97/44326); depression (WO 97/44326); benign prostatic hyperplasia (U.S. Pat. No. 5,952,331); sleep disorder (WO 97/44326); panic disorder, obsessive compulsive disorder, alcoholism, hypertension, anorexia nervosa, and priapism (WO 97/44326); asthma and obstructive airway disease (U.S. Pat. No. 5,952,331); incontinence and bladder dysfunction (WO 96/24351); disorders of the uterus, such as dysmenorrhoea, pre-term labour, post-partum remodelling, endometriosis and fibrosis; pulmonary hypertension (Launay, J. M., et al., Nature Medicine, 8(10), 1129-1135 (2002)).

WO 97/44326 describes aryl pyrimidine derivatives and their use as selective 5-HT_{2B} antagonists. However, although this application discloses a number of compounds, it is desirable to find further classes of compounds to act as 5-HT_{2B} antagonists, which are preferably selective against 5-HT_{2A} and 5-HT_{2C} receptors.

The present inventors have previously described such compounds in co-pending applications PCT/GB2003/000567 and PCT/GB2003/000552, filed 11 Feb. 2003 and U.S. Ser. No. 10/364,672, filed 12 Feb. 2003, which are all incorporated herein by reference.

### SUMMARY OF THE INVENTION

A first aspect of the present invention provides the use of a compound of formula I:

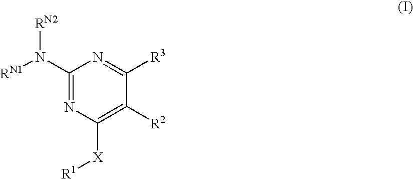

or a pharmaceutically acceptable salt thereof, in the preparation of a medicament for the treatment of a condition alleviated by antagonism of a 5-HT_{2B} receptor, wherein:

### X is O or NH;

R² and R³ are independently selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl; R¹ is an optionally substituted C₉₋₁₄ aryl group or an optionally substituted C₅₋₇ aryl group (which includes an optionally substituted bi-C₅₋₇ aryl group);
R^{N1} and R^{N2} are either:
(i) independently selected from H, R, R′, SO₂R′, C(═O)R, (CH₂)ₙNR^{N3}R^{N4}, where n is from 1 to 4 and R^{N3} and R^{N4} are independently selected from H and R, where R is optionally substituted C₁₋₄ alkyl, and R′ is optionally substituted phenyl-C₁₋₄ alkyl, or
(ii) together with the nitrogen atom to which they are attached, form an optionally substituted C₅₋₇ heterocyclic group.

A second aspect of the present invention provides a method of treating a condition which can be alleviated by antagonism of a 5-HT_{2B} receptor, which method comprises administering to a patient in need of treatment an effective amount of a compound of formula I as defined in the first aspect, or a pharmaceutically acceptable salt thereof.

Conditions which can be alleviated by antagonism of a 5-HT_{2B} receptor are discussed above, and particularly include disorders of the GI tract.

A third aspect of the present invention provides the use of a compound of formula I as defined in the first aspect or a pharmaceutically acceptable salt thereof in a method of therapy, with the proviso that when R^{N1}, R^{N2} and R² are H, R³ is methyl, and X is NH, then R¹ is not: phenyl; 3-1,4-Me-phenyl; 3,5-diacetyl-phenyl, 3-acetyl-phenyl; 4-acetyl-phenyl; and 2-carboxy-phenyl.

A fourth aspect of the present invention provides a pharmaceutical composition comprising a compound of formula I as defined in the first aspect or a pharmaceutically acceptable salt thereof together with a pharmaceutically acceptable carrier or diluent, with the proviso that when R^{N1}, R^{N2} and R² are H, R³ is methyl, and X is NH, then R¹ is not: phenyl; 3-I, 4-Me-phenyl; 3,5-diacetyl-phenyl, 3-acetyl-phenyl; 4-acetyl-phenyl; and 2-carboxy-phenyl.

A fifth aspect of the present invention provides a compound of formula I as defined in the first aspect, except that R¹ can be an optionally substituted C₉₋₁₄ aryl group or an optionally substituted bi-C₅₋₇ aryl group, or a salt, solvate and chemically protected form thereof, with the proviso that when R^{N1}, R^{N2} and R² are H, R³ is methyl, and X is NH, then R¹ is not:

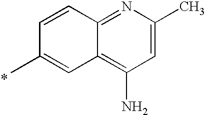

It is preferred that the compounds described above are selective as against 5-HT_{2A} and 5-HT_{2C} receptors.

A sixth aspect of the present invention provides the use of a compound of formula II:

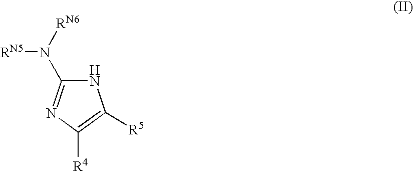

or a pharmaceutically acceptable salt thereof, in the preparation of a medicament for the treatment of a condition alleviated by antagonism of a 5-HT_{2B} receptor, wherein:
R⁵ is selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl;
R⁴ is an optionally substituted C₉₋₁₄ aryl group or an optionally substituted bi-C₅₋₇ aryl group;
R^{N5} and R^{N6} are either:
(i) independently selected from H, R, R′, SO₂R, C(═O)R, (CH₂)ₙNR^{N7}R^{N8}, where n is from 1 to 4 and R^{N7} and R^{N8} are independently selected from H and R, where R is optionally substituted C₁₋₄ alkyl, and R′ is optionally substituted phenyl-C₁₋₄ alkyl, or
(ii) together with the nitrogen atom to which they are attached, form an optionally substituted C₅₋₇ heterocyclic group.

A seventh aspect of the present invention provides a method of treating a condition which can be alleviated by antagonism of a 5-HT_{2B} receptor, which method comprises administering to a patient in need of treatment an effective amount of a compound of formula II as defined in the sixth aspect, or a pharmaceutically acceptable salt thereof.

Conditions which can be alleviated by antagonism of a 5-HT_{2B} receptor are discussed above, and particularly include disorders of the GI tract.

An eighth aspect of the present invention provides the use of a compound of formula II as defined in the sixth aspect, with the proviso that when R^{N5}, R^{N6} and R⁵ are H, R⁴ is not unsubstituted 2-naphthyl or unsubstituted 4-phenyl-phenyl, or a pharmaceutically acceptable salt thereof, in a method of therapy.

A ninth aspect of the present invention provides a pharmaceutical composition comprising a compound of formula II as defined in the eighth aspect or a pharmaceutically acceptable salt thereof together with a pharmaceutically acceptable carrier or diluent.

A tenth aspect of the present invention provides a compound of formula II as defined in the sixth aspect or a salt, solvate and chemically protected form thereof, with the proviso that when R^{N5}, R^{N6} and R⁵ are H, R⁴ is not unsubstituted 1- or 2-naphthyl or unsubstituted 4-phenyl-phenyl.

It is preferred that the compounds described above are selective as against 5-HT_{2A} and 5-HT_{2C} receptors.

An eleventh aspect of the present invention provides the use of a compound of formula IIIa or IIIb:

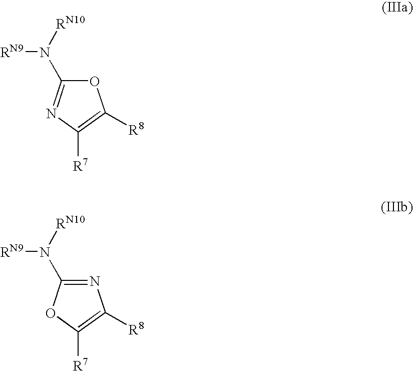

or a pharmaceutically acceptable salt thereof, in the preparation of a medicament for the treatment of a condition alleviated by antagonism of a 5-HT_{2B} receptor, wherein:
R⁸ is selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl;
R⁷ is an optionally substituted bi-C₅₋₇ aryl group;
R^{N9} and R^{N10} are either:
(i) independently selected from H, R, R′, SO₂R, C(═O)R, (CH₂)ₙNR^{N11}R^{N12}, where n is from 1 to 4 and R^{N11} and R^{N12} are independently selected from H and R, where R is optionally substituted C₁₋₄ alkyl, and R′ is optionally substituted phenyl-C₁₋₄ alkyl, or
(ii) together with the nitrogen atom to which they are attached, form an optionally substituted C₅₋₇ heterocyclic group.

A twelfth aspect of the present invention provides a method of treating a condition which can be alleviated by antagonism of a 5-HT_{2B} receptor, which method comprises administering to a patient in need of treatment an effective amount of a compound of formula I as defined in the eleventh aspect, or a pharmaceutically acceptable salt thereof.

Conditions which can be alleviated by antagonism of a 5-HT_{2B} receptor are discussed above, and particularly include disorders of the GI tract.

A thirteenth aspect of the present invention provides the use of a compound of formula IIIa or IIIb as defined in the eleventh aspect, or a pharmaceutically acceptable salt thereof, in a method of therapy.

A fourteenth aspect of the present invention provides a pharmaceutical composition comprising a compound of formula IIIa or IIIb as defined in the eleventh aspect, or a pharmaceutically acceptable salt thereof, together with a pharmaceutically acceptable carrier or diluent.

A fifteenth aspect of the present invention provides a compound of formula IIIa or IIIb as defined in the eleventh aspect, or a salt, solvate and chemically protected form thereof, with the proviso that in formula IIIb, when R^{N9}, R^{N10} and R⁸ are H, R⁷ is not 4-phenyl-phenyl.

It is preferred that the compounds described above are selective as against 5-HT_{2A} and 5-HT_{2C} receptors.

A sixteenth aspect of the present invention provides a compound of formula IVa or IVb:

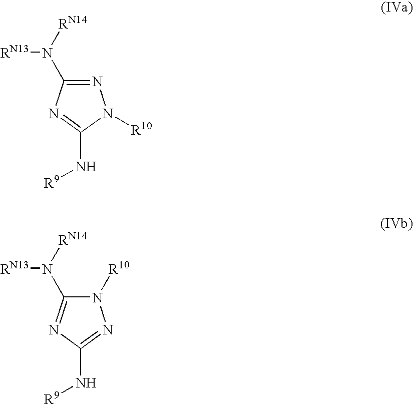

or a salt, solvate and chemically protected form thereof, wherein:
R¹⁰ is selected from the group consisting of H and optionally substituted C₁₋₆ alkyl;
R⁹ is an optionally substituted C₉₋₁₄ aryl group or an optionally substituted bi-C₅₋₇ aryl group;
R^{N13} and R^{N14} are either:
(i) independently selected from H, R, R′, SO₂R, C(═O)R, (CH₂)ₙNR^{N15}R^{N16}, where n is from 1 to 4 and R^{N15} and R^{N16} are independently selected from H and R, where R is optionally substituted C₁₋₄ alkyl, and R′ is optionally substituted phenyl-C₁₋₄ alkyl, or
(ii) together with the nitrogen atom to which they are attached, form an optionally substituted C₅₋₇ heterocyclic group.

A seventeenth aspect of the present invention provides the use of a compound of formula IVa or IVb as defined in the sixteenth aspect or a pharmaceutically acceptable salt thereof in a method of therapy.

An eighteenth aspect of the present invention provides a pharmaceutical composition comprising a compound of formula IVa or IVb as defined in the sixteenth aspect or a pharmaceutically acceptable salt thereof together with a pharmaceutically acceptable carrier or diluent.

A nineteenth aspect of the present invention provides the use of a compound of formula IVa or IVb as defined in the sixteenth aspect or a pharmaceutically acceptable salt thereof in the preparation of a medicament for the treatment of a condition alleviated by antagonism of a 5-HT_{2B} receptor.

A twentieth aspect of the present invention provides a method of treating a condition which can be alleviated by antagonism of a 5-HT_{2B} receptor, which method comprises administering to a patient in need of treatment an effective amount of a compound of formula IVa or IVb as defined in the sixteenth aspect, or a pharmaceutically acceptable salt thereof.

Conditions which can be alleviated by antagonism of a 5-HT_{2B} receptor are discussed above, and particularly include disorders of the GI tract.

It is preferred that the compounds described above are selective as against 5-HT_{2A} and 5-HT_{2C} receptors.

### DEFINITIONS

C₁₋₆ alkyl group: The term “C₁₋₆ alkyl”, as used herein, pertains to a monovalent moiety obtained by removing a hydrogen atom from a carbon atom of a non-cyclic hydrocarbon compound having from 1 to 6 carbon atoms, and which may be saturated or unsaturated.

Examples of saturated C₁₋₆ alkyl groups include methyl (C₁); ethyl (C₂); propyl (C₃), which may be linear (n-propyl) or branched (iso-propyl); butyl (C₄), which may be linear (n-butyl) or branched (iso-butyl, sec-butyl and tert-butyl); pentyl (C₅), which may be linear (n-pentyl, amyl) or branched (iso-pentyl, neo-pentyl); hexyl (C₆), which may be linear (n-hexyl) or branched.

Examples of unsaturated C₁₋₆ alkyl groups, which may be referred to as C₁₋₆ alkenyl (if they included a double bond) or C₁₋₆ alkynyl (if they include a triple bond) groups, include ethenyl(vinyl, —CH═CH₂), ethynyl(ethinyl, —C≡CH), 1-propenyl(—CH═CH—CH₃), 2-propenyl(allyl, —CH—CH═CH₂), 2-propynyl(propargyl, —CH₂—C≡CH), isopropenyl(—C(CH₃)═CH₂), butenyl(C₄), pentenyl(C₅), and hexenyl(C₆).

C₃₋₇ Cycloalkyl: The term “C₃₋₇ cycloalkyl”, as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 ring atoms

Examples of saturated cycloalkyl groups include, but are not limited to, those derived from: cyclopropane (C₃), cyclobutane (C₄), cyclopentane (C₅), cyclohexane (C₆), and cycloheptane (C₇).

Examples of unsaturated cylcoalkyl groups include, but are not limited to, those derived from: cyclobutene (C₄), cyclopentene (C₅), cyclohexene (C₆), and cycloheptene (C₇)

C₃₋₇ cycloalkyl-C₁₋₄ alkyl: The term “C₃₋₇ cycloalkyl-C₁₋₄ alkyl”, as used herein, pertains to a monovalent moiety obtained by removing a hydrogen atom from a carbon atom of a non-cyclic hydrocarbon compound having from 1 to 4 carbon atoms (C₁₋₄ alkyl), which may be saturated or unsaturated, which itself is substituted by a C₃₋₇ cycloalkyl group.

Examples of C₃₋₇ cycloalkyl-C₁₋₄ alkyl groups include, but are not limited to, those derived from: cyclohexylethane (C₆-C₂) and cyclopentylpropene (C₅-C₃).

Phenyl-C₁₋₄ alkyl: The term “phenyl-C₁₋₄ alkyl”, as used herein, pertains to a monovalent moiety obtained by removing a hydrogen atom from a carbon atom of a non-cyclic hydrocarbon compound having from 1 to 4 carbon atoms (C₁₋₄ alkyl), which may be saturated or unsaturated, which itself is substituted by a phenyl group (C₆H₅—).

Examples of phenyl-C₁₋₄ alkyl groups include, but are not limited to, benzyl(phenyl-CH₂—) and those derived from: phenylethane (phenyl-C₂) and phenylpropane (phenyl-C₃).

C₅₋₇ Heterocyclyl: The term “C₅₋₇ heterocyclyl”, as used herein, pertains to a monovalent moiety obtained by removing a hydrogen atom from a ring atom of a heterocyclic compound, which moiety has from 5 to 7 ring atoms, of which from 1 to 4 are ring heteroatoms. In particular, when R² and R³ together with the nitrogen atom to which they are attached form a C₅₋₇ heterocyclic ring, at least one ring atom will be nitrogen.

Examples of C₅₋₇ heterocyclyl groups having at least one nitrogen atom, include, but are not limited to, those derived from:

GN₁: pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇); N₂: imidazolidine (C₅), pyrazolidine (diazolidine) (C₅), imidazoline (C₅), pyrazoline (dihydropyrazole) (C₆), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
N₁O₁S₁: oxathiazine (C₆).

C₉₋₁₄ Aryl: The term “C₉₋₁₄ aryl”, as used herein, pertains to a monovalent moiety obtained by removing a hydrogen atom from an aromatic ring atom of an aromatic compound with at least two fused rings, which moiety has from 9 to 14 ring atoms. Preferably, each ring has from 5 to 7 ring atoms.

The ring atoms may be all carbon atoms, as in “carboaryl groups” (e.g. C₉₋₁₄ carboaryl).

Examples of carboaryl groups include, but are not limited to, those derived from naphthalene (C₁₀), azulene (C₁₀), anthracene (C₁₄) and phenanthrene (C₁₄).

Examples of aryl groups which comprise fused rings, at least one of which is an aromatic ring, include, but are not limited to, groups derived from indene (C₉), isoindene (C₉) tetralin (C₁₀) and fluorene (C₁₃).

Alternatively, the ring atoms may include one or more heteroatoms, as in “heteroaryl groups” (e.g. C₉₋₁₄ heteroaryl).

Examples of heteroaryl groups, include, but are not limited to:
C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g. adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiophene (S₁), benzothiazole (N₁S₁), benzothiadiazole (N₂S);
C₁₀ heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ heteroaryl groups (with 2 fused rings) derived from benzoazepine (N₁), 5-oxa-9-aza-benzocycloheptene (N₁O₁);
C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,
C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above described C₉₋₁₄ aryl group includes the radical formed by removal of a hydrogen atom from any of the possible aromatic ring atoms. The groups formed by this removal can be described by the number of the ring atom from which the hydrogen is removed, if there is more than one possibility. The carboaryl groups derived from, for example, naphthalene (C₁₀) can be either napth-1-yl or nath-2-yl; and from azulene (C₁₀) can be azul-1-yl, azul-2-yl, azul-4-yl, azul-5-yl and azul-6-yl. The heteroaryl groups derived, for example, from isoquinoline can be isoquinol-x-yl α-isoquinolyl), where x can be 1, 3, 4, 5, 6, 7 or 8.

Bi—C₅₋₇ aryl: The term “Bi—C₅₋₇ aryl”, as used herein, pertains to a monovalent moiety obtained by removing a hydrogen atom from an aromatic ring atom of an aromatic compound with two aromatic rings, where each ring has from 5 to 7 ring atoms, and the rings are linked by a single bond.

If the ring atoms of an aromatic ring are all carbon atoms, as in a “carboaryl ring”, then that ring will be derived from benzene.

One or more of the ring atoms may be a heteroatom, as in a “heteroaryl ring”. Examples of heteroaryl rings include, but are not limited to:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅)
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₅);
N₃: triazole (C₅), triazine (C₆); and,
N₄: tetrazole (C₅).

The bi-C₅₋₇ aryl group includes the radical formed by removal of a hydrogen atom from any of the possible aromatic ring atoms of the ‘first’ aromatic ring, i.e. the ring from which the hydrogen atom is removed, and the ‘second’ aromatic ring, i.e. the ring from which the hydrogen atom is not removed, may be bonded to the first aromatic ring at any position in relation to the ring atom from which the hydrogen atom has been removed. For example, if both aromatic rings are unsubstituted benzene rings, then the following groups are possible:

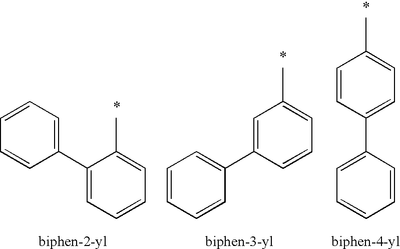

The phrase “optionally substituted”, as used herein, pertains to a parent group, as above, which may be unsubstituted or which may be substituted by one of the following substituent groups:

C₁₋₂₀ alkyl group: The term “C₁₋₂₀ alkyl”, as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated, partially unsaturated, or fully unsaturated. Thus, the term “alkyl” includes the sub-classes alkenyl, alkynyl and cycloalkyl discussed below.

In this context, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl,” as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl.

Examples of saturated alkyl groups include, but are not limited to, methyl(C₁), ethyl(C₂), propyl(C₃), butyl(C₄), pentyl(C₅), hexyl(C₆), heptyl(C₇), octyl(C₈), nonyl(C₉), decyl(C₁₀), n-undecyl(C₁₁), dodecyl(C₁₂), tridecyl(C₁₃), tetradecyl(C₁₄), pentadecyl(C₁₅), and eicodecyl(C₂₀).

Examples of saturated linear alkyl groups include, but are not limited to, methyl(C₁), ethyl(C₂), n-propyl(C₃), n-butyl(C₄), n-pentyl(amyl) (C₅), n-hexyl(C₆)—, and n-heptyl (C₇).

Examples of saturated branched alkyl groups include iso-propyl(C₃), iso-butyl(C₄), sec-butyl(C₄), tert-butyl (C₄), iso-pentyl(C₅), and neo-pentyl(C₅).

Cycloalkyl: The term “cycloalkyl”, as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 20 ring atoms (unless otherwise specified). Preferably, each ring has from 3 to 7 ring atoms.

Examples of saturated cycloalkyl groups include, but are not limited to, those derived from: cyclopropane (C₃), cyclobutane (C₄), cyclopentane (C₅), cyclohexane (C₆), cycloheptane (C₇), norbornane (C₇), norpinane (C₇), norcarane (C₇), adamantane (C₁₀), and decalin (decahydronaphthalene) (C₁₀).

Examples of saturated cycloalkyl groups, which are also referred to herein as “alkyl-cycloalkyl” groups, include, but are not limited to, methylcyclopropyl, dimethylcyclopropyl, methylcyclobutyl, dimethylcyclobutyl, methylcyclopentyl, dimethylcyclopentyl, methylcyclohexyl, and dimethylcyclohexyl, menthane, thujane, carane, pinane, bornane, norcarane, and camphene.

Examples of unsaturated cyclic alkenyl groups, which are also referred to herein as “alkyl-cycloalkenyl” groups, include, but are not limited to, methylcyclopropenyl, dimethylcyclopropenyl, methylcyclobutenyl, dimethylcyclobutenyl, methylcyclopentenyl, dimethylcyclopentenyl, methylcyclohexenyl, and dimethylcyclohexenyl.

Examples of cycloalkyl groups, with one or more other rings fused to the parent cycloalkyl group, include, but are not limited to, those derived from: indene (C₉), indan (e.g., 2,3-dihydro-1H-indene) (C₉), tetraline (1,2,3,4-tetrahydronaphthalene (C₁₀), acenaphthene (C₁₂), fluorene (C₁₃), phenalene (C₁₃), acephenanthrene (C₁₅), aceanthrene (C₁₆). For example, 2H-inden-2-yl is a C₅cycloalkyl group with a substituent (phenyl) fused thereto.

Alkenyl: The term “alkenyl,” as used herein, pertains to an alkyl group having one or more carbon-carbon double bonds.

Examples of groups of alkenyl groups include C₂₋₄ alkenyl, C₂₋₇ alkenyl, C₂₋₂₀ alkenyl.

Examples of alkenyl groups include, but are not limited to, ethenyl(vinyl, —CH═CH₂), 1-propenyl(—CH═CH—CH₃), 2-propenyl(allyl, —CH—CH═CH₂), isopropenyl(—C(CH₃)═CH₂), butenyl(C₄), pentenyl(C₅), and hexenyl(C₆).

Examples of cyclic alkenyl groups, which are also referred to herein as “cycloalkenyl” groups, include, but are not limited to, cyclopropenyl(C₃), cyclobutenyl(C₄), cyclopentenyl(C₅), and cyclohexenyl(C₆).

Alkynyl: The term “alkynyl,” as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of alkynyl groups include, but are not limited to, ethynyl(ethinyl, —C≡CH) and 2-propynyl(propargyl, —CH₂—C≡CH).

C₃₋₂₀ heterocyclyl group: The term “C₃₋₂₀ heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆ heterocyclyl,” as used herein, pertains to a heterocyclyl group having 5 or 6 ring atoms. Examples of groups of heterocyclyl groups include C₃₋₂₀ heterocyclyl, C₃₋₇ heterocyclyl, C₅₋₇ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene)(C₅), thiane (tetrahydrothiopyran) (C₆), thiepane (C₇);
O₂: dioxolane (C₅), dioxane (C₆), and dioxepane (C₇);
O₂: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

C₅₋₂₀ Aryl: The term “C₅₋₂₀ aryl,” as used herein, pertains to a monovalent moiety obtained by removing a hydrogen atom from an aromatic ring atom of an aromatic compound, which moiety has from 5 to 20 ring atoms (unless otherwise specified). Preferably, each ring has from 5 to 7 ring atoms. The term “C₅₋₇ aryl” is a subset of the term “C₅₋₂₀ aryl” and refers to monovalent moieties obtained by removing a hydrogen atom from an aromatic compound which has from 5 to 7 ring atoms.

The ring atoms may be all carbon atoms, as in “carboaryl groups.” Examples of carboaryl groups include, but are not limited to, those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆).

Alternatively, the ring atoms may include one or more heteroatoms, as in “heteroaryl groups.” Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₃: thiazole (C₅), isothiazole (C₅)
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl groups which comprise fused rings, include, but are not limited to:
C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ heteroaryl groups (with 2 fused rings) derived from benzodiazepine (N₂);
C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,
C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

### Halo: —F, —Cl, —Br, and —I.

### Hydroxy: —OH.

Ether: —OR, wherein R is an ether substituent, for example, a C₁₋₇alkyl group (also referred to as a C₁₋₇alkoxy group, discussed below), a C₃₋₂₀heterocyclyl group (also referred to as a C₃₋₂₀heterocyclyloxy group), or a C₅₋₂₀aryl group (also referred to as a C₅₋₂₀aryloxy group), preferably a C₁₋₇alkyl group.

C₁₋₇alkoxy: —OR, wherein R is a C₁₋₇alkyl group. Examples of C₁₋₇alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Oxo(keto, -one): ═O.
Thione (thioketone): ═S.

Imino (imine): ═NR, wherein R is an imino substituent, for example, hydrogen, C₁₋₇alkyl group, a C₃₋₂₀heterocyclyl group, or a C₅₋₂₀aryl group, preferably hydrogen or a C₁₋₇alkyl group. Examples of imino groups include, but are not limited to, ═NH, ═NMe, ═NEt, and ═NPh.

Formyl(carbaldehyde, carboxaldehyde): —C(═O)H.

Acyl(keto): —C(═O)R, wherein R is an acyl substituent, for example, a C₁₋₇alkyl group (also referred to as C₁₋₇alkylacyl or C₁₋₇alkanoyl), a C₃₋₂₀heterocyclyl group (also referred to as C₃₋₂₀heterocyclylacyl), or a C₅₋₂₀aryl group (also referred to as C₅₋₂₀arylacyl), preferably a C₁₋₇alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy(carboxylic acid): —C(═O)OH.
Thiocarboxy(thiocarboxylic acid): —C(═S)SH.
Thiolocarboxy(thiolocarboxylic acid): —C(═O)SH.
Thionocarboxy(thionocarboxylic acid): —C(═S)OH.
Imidic acid: —C(═NH)OH.
Hydroxamic acid: —C(═NOH)OH.

Ester (carboxylate, carboxylic acid ester, oxycarbonyl): —C(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.

Acyloxy(reverse ester): —OC(—O)R, wherein R is an acyloxy substituent, for example, a C₁₋₇alkyl group, a C₃₋₂₀heterocyclyl group, or a C₅₋₂₀aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O) Ph, and —OC(═O)CH₂Ph.

Oxycarbonyloxy: —OC(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —OC(═O)OCH₃, —OC(═O)OCH₂CH₃, —OC(═O)OC(CH₃)₃, and —OC(═O)OPh.

Carbamate: —OC(═O)NR¹R², wherein R¹ and R² are independently amino substituents, as defined for amino groups. Examples of carbamate groups include, but are not limited to, —OC(═O)NH₂, —OC(═O)NHCH₃, —OC(═O)N(CH₃)₂, —OC(═O)NHCH₂CH₃, and —OC(═O)N(CH₂CH₃)₂.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, hydrogen, a C₁₋₇alkyl group, a C₃₋₂₀heterocyclyl group, or a C₅₋₂₀aryl group, preferably hydrogen or a C₁₋₇alkyl group, and R² is an acyl substituent, for example, a C₁₋₇alkyl group, a C₃₋₂₀heterocyclyl group, or a C₅₋₂₀aryl group, preferably hydrogen or a C₁₋₇alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

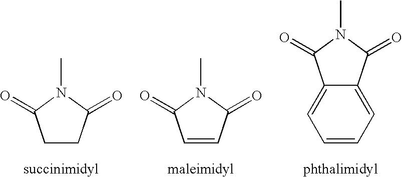

Thioamido (thiocarbamyl): —C(═S)NR¹R², wherein R¹ and R² are independently amino substituents, as defined for amino groups. Examples of thioamido groups include, but are not limited to, —C(═S)NH₂, —C(═S)NHCH₃, —C(═S)N(CH₃)₂, and —C(═S)NHCH₂CH₃.

Ureido: —N(R¹)CONR²R³ wherein R² and R³ are independently amino substituents, as defined for amino groups, and R¹ is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂

### Guanidino: —NH—C(═NH)NH₂.

Tetrazolyl: a five membered aromatic ring having four nitrogen atoms and one carbon atom,

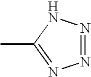

Amino: —NR¹R², wherein R¹ and R² are independently amino substituents, for example, hydrogen, a C₁₋₇alkyl group (also referred to as C₁₋₇alkylamino or di-C₁₋₇alkylamino), a C₃₋₂₀heterocyclyl group, or a C₅₋₂₀aryl group, preferably H or a C₁₋₇alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amidine (amidino): —C(═NR)NR₂, wherein each R is an amidine substituent, for example, hydrogen, a C₁₋₇alkyl group, a C₃₋₂₀heterocyclyl group, or a C₅₋₂₀aryl group, preferably H or a C₁₋₇alkyl group. Examples of amidine groups include, but are not limited to, —C(═NH)NH₂, —C(═NH)NMe₂, and —C(═NMe)NMe₂.

### Nitro: —NO₂.

### Nitroso: —NO.

Cyano(nitrile, carbonitrile): —CN.
Sulfhydryl(thiol, mercapto): —SH.

Thioether (sulfide): —SR, wherein R is a thioether substituent, for example, a C₁₋₇alkyl group (also referred to as a C₁₋₇alkylthio group), a C₃₋₂₀heterocyclyl group, or a C₅₋₂₀aryl group, preferably a C₁₋₇alkyl group. Examples of C₁₋₇alkylthio groups include, but are not limited to, —SCH₃ and —SCH₂CH₃.

Disulfide: —SS—R, wherein R is a disulfide substituent, for example, a C₁₋₇alkyl group, a C₃₋₂₀heterocyclyl group, or a C₅₋₂₀aryl group, preferably a C₁₋₇alkyl group (also referred to herein as C₁₋₇alkyl disulfide). Examples of C₁₋₇alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇alkyl group, a C₃₋₂₀heterocyclyl group, or a C₅₋₂₀aryl group, preferably a C₁₋₇alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇alkyl group, a C₃₋₂₀ heterocyclyl group, or a C₅₋₂₀aryl group, preferably a C₁₋₇ alkyl group, including, for example, a fluorinated or perfluorinated C₁₋₇alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl(tosyl), 4-chlorophenylsulfonyl (closyl), 4-bromophenylsulfonyl(brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.
Sulfonic acid (sulfo): —S(═O)₂OH, —SO₃H.

Sulfinate (sulfinic acid ester): —S(═O)OR; wherein R is a sulfinate substituent, for example, a C₁₋₇alkyl group, a C₃₋₂₀heterocyclyl group, or a C₅₋₂₀aryl group, preferably a C₁₋₇alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇alkyl group, a C₃₋₂₀heterocyclyl group, or a C₅₋₂₀aryl group, preferably a C₁₋₇alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇alkyl group, a C₃₋₂₀heterocyclyl group, or a C₅₋₂₀aryl group, preferably a C₁₋₇alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇alkyl group, a C₃₋₂₀heterocyclyl group, or a C₅₋₂₀aryl group, preferably a C₁₋₇alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇alkyl group, a C₃₋₂₀heterocyclyl group, or a C₅₋₂₀aryl group, preferably a C₁₋₇alkyl group. Examples of sulfate groups include, but are not limited to, —OS(═O)₂OCH₃ and —SO(═O)₂OCH₂CH₃.

Sulfamyl(sulfamoyl; sulfinic acid amide; sulfinamide): —S(═O)NR¹R¹, wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R³ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃) S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇alkyl group, a C₃₋₂₀heterocyclyl group, or a C₅₋₂₀aryl group, preferably a C₁₋₇alkyl group. Examples of sulfinamino groups include, but are not limited to, —NHS(═O)CH₃ and —N(CH₃)S(═O)C₆H₅.

The above listed substituent groups, may themselves be further substituted, where appropriate, by one or more of themselves.

### Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Isomers, Salts, Solvates and Protected Forms Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; α- and β-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

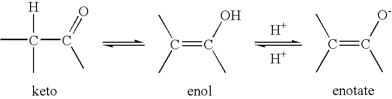

Tautomeric forms of particular relevance to the present invention include those of formula II, as illustrated below:

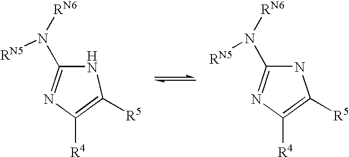

Note that specifically included in the term “isomer” are compounds with one or more isotopic substitutions. For example, H may be in any isotopic form, including ¹H, ²H (D), and ³H(T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc. It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

A wide variety of such “protecting”, “blocking”, or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl(diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇-trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. Suitable dose ranges will typically be in the range of from 0.01 to 20 mg/kg/day, preferably from 0.1 to 10 mg/kg/day.

### Compositions and Their Administration

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium carbonate, and the like. Such compositions take the form of solutions, suspensions, tablets, pills, capsules, powders, sustained release formulations and the like. Such compositions may contain 1-95% active ingredient, more preferably 2-50%, most preferably 5-8.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

### Acronyms

For convenience, many chemical moieties are represented using well known abbreviations, including but not limited to, methyl (Me), ethyl (Et), n-propyl (nPr), iso-propyl (iPr), n-butyl (nBu), sec-butyl (sBu), iso-butyl (iBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), acetonitrile (ACN), trifluoroacetic acid (TFA), dimethylformamide (DMF), tetrahydrofuran (THF), and dimethylsulfoxide (DMSO).

### General Synthesis Methods

Compounds of formula I can be synthesised by the following route:

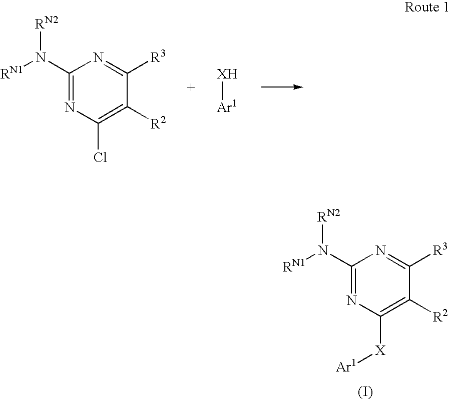

wherein R², R³, R^{N1} and R^{N2} are as defined above, and Ar¹ is either R¹, as defined above (i.e. an optionally substituted C₉₋₁₄ aryl group or an optionally substituted C₅₋₇ aryl group, which includes an optionally substituted bi-C₅₋₇ aryl group) or the first aromatic ring of the bi-C₅₋₇ aryl group with a moiety for attaching the second aromatic ring of the bi-C₅₋₇ aryl group. In the latter case, the method of route 1 is followed by a further step of joining the second aromatic ring of the bi-C₅₋₇ aryl group to the first aromatic ring.

The method of route 1 is carried out in solution (for example, aqueous) optionally in the presence of base with heating (for example, microwave heating).

If Ar¹ is only the first aromatic ring of the bi-C₅₋₇ aryl group, then it preferably bears either:

i) a halogen, such as bromo, iodo or chloro, or a group which is subsequently converted into a triflic group, for example a protected alcohol; or (ii) a group, such as bromo or iodo, which is subsequently converted into, for example, a boronic acid group or derivative thereof, or certain magnesium, tin or zinc containing organometallic reagents.

The second aromatic ring of the bi-C₅₋₇ aryl group bears the other of the final groups of (i) and (ii) above, such that the two rings may be joined by a palladium catalysed coupling reaction. The palladium catalyst may be tetrakis(triphenylphosphine)palladium(0), and the reaction may be carried out in the presence of an inorganic base, such as sodium carbonate. The reaction is usually carried out by heating at about 80-90° C. for several hours.

Compounds of formula II, where R^{NS} and R^{N6} are H, can be synthesised by the following route:

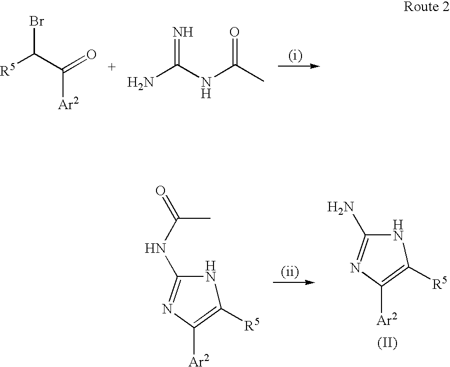

wherein R⁵ is as defined above, and Ar² is either R⁴, as defined above (i.e. an optionally substituted C₉₋₃₄ aryl group or an optionally substituted bi-C₅₋₇ aryl group) or the first aromatic ring of the bi-C₅₋₇ aryl group with a moiety for attaching the second aromatic ring of the bi-C₅₋₇ aryl group. In the latter case, the method of route 2 includes a further step of joining the second aromatic ring of the bi-C₅₋₇ aryl group to the first aromatic ring. This further step may occur between steps (i) and (ii), or after step (ii).

Step (i) is usually carried out by heating the two reactants in organic solvent (for example, DMF). The second step, which is the removal of the acetyl group is carried out under standard conditions, for example, in a 5:1 mixture of industrial methylated spirits and water in the presence of concentrated sulfuric acid, followed by basification.

If Ar² is only the first aromatic ring of the bi-C₅₋₇ aryl group, then its preferred substituents and method of joining the second aromatic ring are as above for Ar¹.

Compounds of formula IIIa and IIIb where at least one of R^{N9} and R^{N10} is hydrogen can be synthesised following the route disclosed by Cockerill (Cockerill, A. F., et al., Synthesis, 1976, 591-593 which is incorporated herein by reference).

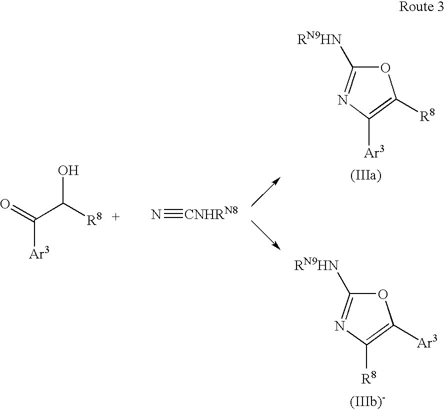

where R⁸ and R^{N9} are as defined), and Ar³ is either R⁷, as defined above (i.e. an optionally substituted bi-C₅₋₇ aryl group) or the first aromatic ring of the bi-C₅₋₇ aryl group with a moiety for attaching the second aromatic ring of the bi-C₅₋₇ aryl group. In the latter case, the method of route 3 includes a further step of joining the second aromatic ring of the bi-C₅₋₇ aryl group to the first aromatic ring. In this method the 2-amino oxazole is produced by the condensation of the appropriate α-hydroxy ketone with cyanamide or alkylcyanamide, which reaction can be carried out in aqueous solution or in the presence of a mineral acid or a base catalyst (e.g. sodium hydroxide).

The inventors have found that product of the reaction may be either the 2-amino-4-aryl oxazole, the 2-amino-5-aryl oxazole, or a mixture of the two, with the 2-amino-5-aryl oxazole being favoured. It is thought that carrying the reaction out under milder conditions may increase the amount of the 2-amino-4-aryl oxazole produced.

If the product of the method is a mixture of compounds of formula IIIa and IIIb these may be separated by column chromatography.

Without wishing to be bound by theory, the product of formula IIIb results from the reaction of the tautomeric form of the starting material:

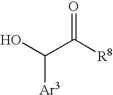

The two tautometric forms of the starting material exist in equilibrium, which under the conditions of the reaction tends to favour the formation of IIIb rather than IIIa.

The starting α-hydroxyketones can be synthesised via α-bromo and α-acetoxy intermediates, some of which are commercially available, from the parent ketones.

The substitution on the 2-amino group can be introduced using a substituent on the cyanamide, or may be introduced later in the reaction scheme, again with, if necessary, protection of other functional groups in the molecule.

The compounds of formula IIIb when R^{N9} and R^{N10} represent hydrogen may also be obtained regio-specifically be reacting an α-bromoketone with cyanamide in ethanol in the presence of sodium ethoxide and proceeds via a cyano α-aminoketone, as shown in Route 4:

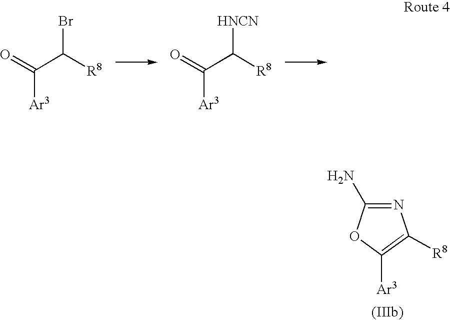

Compounds of formula IIIb where R⁸ is hydrogen and R^{N9} and R^{N10} are hydrogen or an alkyl group may also be prepared by a stereoselective method described by van Leusen, et al., J. Org. Chem., 46, 2069-2072 (1981), which is incorporated herein by reference, that employs the reaction of an N-tosylmethylcarbodiimide with an aromatic aldehyde in a solvent, such as methylene chloride, in the presence of a base (e.g. aqueous sodium hydroxide) and a phase transfer catalyst (e.g. tetrabutylammonium bromide), as shown in Route 5. For compounds where R^{N9} is hydrogen, the group R^{N9} in the carbodiimide is a trityl group that is removed after condensing with the aldehyde by treatment with mineral acid.

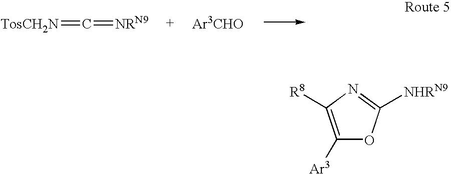

Compounds of formula IIIa can be prepared by following the route (Route 6) described by Gompper, R., and Christmann, O., Chem. Ber. 92, 1944-1949 (1959), which is incorporated herein by reference, in which the 2-amino or 2-alkylamino oxazole is produced by condensing the appropriate α-bromo ketone with urea or substituted urea, which reaction is carried out in an organic solvent, e.g. dimethylformamide.

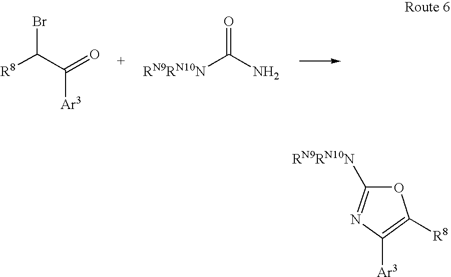

The 5-substituent on the oxazole ring is present in the starting material as the alkyl chain of the α-bromo alkylarylketone, which can be obtained from the parent alkylarylketone if necessary.

This route can be used for compounds of formula IIIa where R⁷ is an optionally substituted C₉₋₁₄ aryl group and R^{N9} and R^{N10} are hydrogen or alkyl groups but is less preferred for these compounds.

The starting ketones for both routes are either commercially available or accessible by, for example, Grignard reactions on the corresponding nitrites or Friedal Crafts reaction of substituted aryls.

A further method of preparing compounds of formula IIIa and IIIb respectively is by a palladium catalysed coupling reaction of a 2-amino-4-substituted oxazole or 2-amino-5-substituted oxazole with an aryl boronic acid, or derivative thereof. The 4- or 5-substituent on the oxazole ring may typically be a halogen, such as bromo, iodo or chloro, or a group such as trifluoromethanesulfonate or a phosphate ester. The aryl boronic acid may also be replaced by certain magnesium, tin or zinc containing organometallic reagents. For example, a 2-amino-4-bromo-oxazole may be reacted with an aryl boronic acid derivative in an aqueous solvent, for example a mixture of ethanol, water and dimethoxyethane, containing a palladium catalyst such as tetrakis(triphenylphosphine)palladium(0) and an inorganic base such as sodium carbonate. The reaction is carried out by heating at about 80-90° C. for several hours.

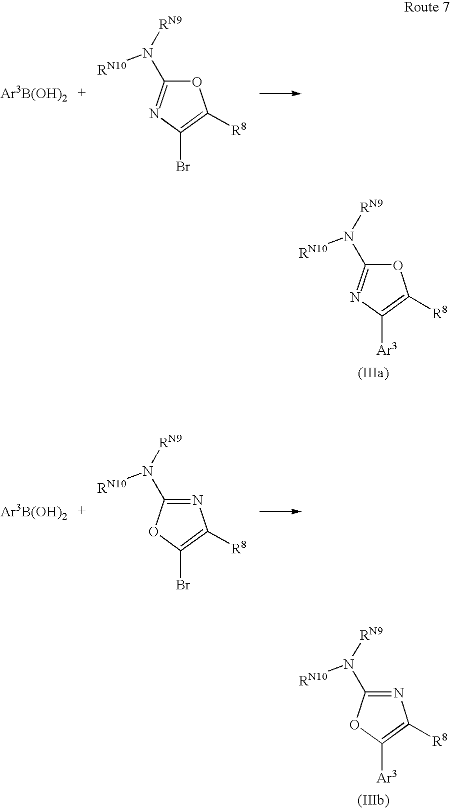

Alternatively, the boronic acid residue, or equivalent, may be on the 4-position of the oxazole ring and the halogen, or equivalent, on the aryl group.

If Ar³ in the above route represents only the first aromatic ring of the bi-C₅₋₇ aryl group, then appropriate protection, or the use of precursor groups, may be required to prevent unwanted side reactions.

Compounds of formulae IIIa and IIIb may also be prepared by nucleophilic displacement of the intermediate chloro compounds with ammonia or amines as described, for example, by Marchetti, E., et al., J. Med. Chem., 11, 1092-1093 (1968), which are incorporated herein by reference.

Compounds of formulae IVa and IVb, where R^{N13} and R^{N14} are both hydrogen, may be synthesised by the following route following that described in Reiter, J. and Pongo, L., J. Het. Chem., 23, 401-408 (1986), which is incorporated herein by reference:

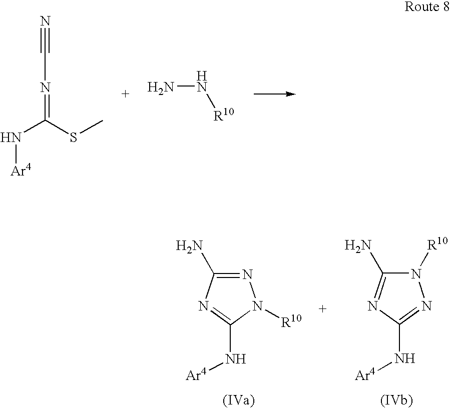

where A/r⁴ is either R⁷, as defined above (i.e. an optionally substituted C₉₋₁₄ aryl group or an optionally substituted bi-C₅₋₇ aryl group) or the first aromatic ring of the bi-C₅₋₇ aryl group with a moiety for attaching the second aromatic ring of the bi-C₅₋₇ aryl group. In the latter case, the method of route 2 includes a further step of joining the second aromatic ring of the bi-C₅₋₇ aryl group to the first aromatic ring.

If the reaction does result in a mixture of a compound of formula IVa and a compound of formula IVb, then these may be separated using, for example, column chromatography.

In any of the above routes, any substitution on the C₉₋₁₄ aryl group or bi-C₅₋₇ aryl group is preferably present in the relevant starting material, but could be introduced later in the reaction scheme, with, if necessary, appropriate protection of other functional groups present in the molecule. Derivation of the amino group attached to the central ring of the compound is possible to provide varied groups at that position.

### Preferences

The following preferences may be combined with one another, and may be different for each aspect of the present invention.

The optional substituents for all groups are preferably independently selected from halo, hydroxy, alkoxy (more preferably C₁₋₄ alkoxy), amino (more preferably NH₂, C₁₋₄ alkyl amino, C₁₋₄ dialkyl amino), and amido (more preferably CONH₂, C₁₋₄ alkyl amido, C₁₋₄ dialkyl amido)

### Pyrimidines

R^{N1} and R^{N2}

In some embodiments it is preferred that both R^{N1} and R^{N2} are substituted, and in other embodiments that only one or neither of R^{N1} and R^{N2} are substituted. Each of R^{N1} and R^{N2} are preferably independently selected from H, R, R′v, where R and R′ are as defined above, and more preferably selected from H and R. R is preferably an optionally substituted C₁₋₄ alkyl group. The preferred substituents for R and R′ include halo, hydroxy, amino and acetyl. R^{N1} and R^{N2} are more preferably independently selected from H and methyl, and are most preferably both H.

### R²

R² is preferably selected from H, optionally substituted C₁₋₆ alkyl and optionally substituted C₃₋₇ cycloalkyl, more preferably from H and unsubstituted C₁₋₆ alkyl (preferably methyl) and is most preferably H.

### R³

R³ is preferably selected from H, optionally substituted C₁₋₆ alkyl and optionally substituted C₃₋₇ cycloalkyl(especially when X is NH), more preferably from H and optionally substituted C₁₋₆ alkyl (preferably methyl and ethyl) and is most preferably methyl.

### X

X is preferably NH.

### R¹

R¹ is preferably an optionally substituted C₉₋₁₄ aryl group (more preferably naphthyl) or an optionally substituted bi-C₅₋₇ aryl group (more preferably bi-C₆ aryl, most preferably bi-phenyl). This preference for R¹ is especially preferred when R^{N1}, R^{N2} and R² are H, R³ is methyl and X is NH.

If R¹ is an optionally substituted C₅₋₇ aryl group (preferably phenyl), then it preferably bears an halo group at the meta position, and may be further substituted, in particular with halo groups.

If R¹ is an optionally substituted C₅₋₇ aryl group, then it is preferred that is not substituted by a carbonyl based group, for example amido. It is also preferred that the sole substituent is not in the ortho position.

If X is O, then it is preferred that R¹ is a C₉₋₁₄ aryl group or a bi-C₅₋₇ aryl group, where the second aryl group is meta to the first.

If R¹ is an optionally substituted bi-C₅₋₇ aryl group, then preferred substituents include, but are not limited to, C₁₋₄ alkyl (preferably methyl), hydroxy, C₁₋₄ alkoxy (preferably methoxy) and NH₂. It is preferred that the substituent is not acylamido or a sulfur based group (e.g. sulfonyl).

If R¹ is an optionally substituted bi-C₅₋₇ aryl group, then it is preferably a bi-C₆ aryl group and is more preferably a bi-phenyl group. Most preferably R¹ is a 3-phenyl-phenyl group. It is preferred that any substituent is on the distal phenyl ring, preferably at the 2-position.

If R¹ is an optionally substituted C₉₋₁₄ aryl group, preferred substituent groups for the C₉₋₁₄ aryl group (especially when X is O) include halo, hydroxy, C₁₋₄ alkoxy, cyano, amino, amido and C₁₋₄ alkyl, of which hydroxy, and C₁₋₄ alkoxy are more preferred. It is also preferred that the C₉₋₁₄ aryl group bears no oxo substituents.

If the C₉₋₁₄ aryl group is a naphth-1-yl group, preferred substituent positions are 2, 4 and 7, with 2 being most preferred. The preferred substituents at the 2-position are hydroxy, C₁₋₄ alkyl and C₁₋₄ alkoxy, with C₁₋₁₄ alkoxy (e.g. methoxy and ethoxy) being most preferred.

### Imidazoles

### R^{N5} and R^{N6}

In some embodiments it is preferred that both R^{N5} and R^{N6} are substituted, and in other embodiments that only one or neither of R^{N5} and R^{N6} are substituted. Each of R^{N5} and R^{N6} are preferably independently selected from H, R, R′ and C(═O)R, where R and R′ are as defined above, and more preferably selected from H, R and C(═O)R. R is preferably an optionally substituted C₁₋₄ alkyl group. The preferred substituents for R and R′ include halo, hydroxy, amino and acetyl. More preferably, at least one of R^{N5} and R^{N6} is H, and the other is selected from H and C(═O)Me. There is a preference for at least one of R^{N5} and R^{N6} to be R, R′, SO₂R, C(═O)R, (CH₂)ₙNR^{N7}R^{N8}, when R⁴ is an unsubstituted 4-phenyl-phenyl group.

### R⁵

R⁵ is preferably selected from H, optionally substituted C₁₋₆ alkyl and optionally substituted C₃₋₇ cycloalkyl, more preferably from H and unsubstituted C₁₋₆ alkyl (preferably methyl, and —C(CH₃)₂) and is most preferably H. There is a preference for R⁵ to be an optionally substituted C₁₋₆ alkyl, C₃₋₇ cycloalkyl, C₃₋₇ cycloalkyl, C₃₋₇ cycloalkyl-C₁₋₄ alkyl and phenyl-C₁₋₄ alkyl, with a further preference for C₁₋₆ alkyl, especially C₁₋₃ alkyl (e.g. methyl, iso-propyl), when R⁴ is an unsubstituted naphthyl group.

### R⁴

R⁴ is preferably an optionally substituted C₉₋₁₄ aryl group or an optionally substituted 3- or 4-C₅₋₆aryl-C₅₋₆ aryl group (for example, 3-phenyl-phenyl and 4-phenyl-phenyl).

R⁴ is preferably optionally substituted C₉₋₁₄ carboaryl group, for example, naphth-1-yl, naphth-2-yl, anthracen-1-yl, anthracen-2-yl, anthracen-9-yl, phenanthren-1-yl, phenanthren-2-yl, phenanthren-3-yl and phenanthren-4-yl, phenanthren-9-yl. Of these napth-1-yl and napth-2-yl are preferred, with naphthy-1-yl being most preferred. Other preferred R⁴ groups include benzo[b]thiophen-2-yl, benzo[b]thiophen-4-yl and benzo[1,4]dioxin-5-yl.

Preferred substituent groups for the C₉₋₁₄ aryl group include halo, hydroxy, C₁₋₄ alkoxy, cyano, amino, amido and C₁₋₄ alkyl, of which hydroxy, fluoro and C₁₋₄ alkoxy are more preferred. It is also preferred that the C₉₋₁₄ aryl group bears no oxo substituents.

If the C₉₋₁₄ aryl group is a naphth-1-yl group, preferred substituent positions are 2, 4 and 7, with 2 being most preferred. The preferred substituents at the 2-position are hydroxy, C₁₋₄ alkyl and C₁₋₄ alkoxy, with C₁₋₄ alkoxy (e.g. methoxy and ethoxy) being most preferred.

### Oxazoles

It is preferred that the compounds are of formula (IIIb).

R⁸ is preferably selected from H and optionally substituted C₁₋₆ alkyl and C₃₋₇ cycloalkyl, more preferably H and optionally substituted C₁₋₆ alkyl. Especially preferred are H, and C₁₋₄ alkyl (e.g. methyl, iso-propyl). In some embodiments the group may be unsubstituted, but when the group is substituted, preferred substituent groups include halo, hydroxy, and amino. Most preferably, R⁸ is H or methyl.

In some embodiments it is preferred that both R^{N9} and R^{N10} are substituted, and in other embodiments that only one or neither of R^{N9} and R^{N10} are substituted. Each of R^{N9} and R^{N10} are preferably independently selected from H, R, R′, where R and R′ are as defined above, and more preferably selected from H and R. R is preferably an optionally substituted C₁₋₄ alkyl group. The preferred substituents for R and R′ include halo, hydroxy, amino and acetyl.

R⁷ is preferably an optionally substituted bi-C₆ aryl group and is more preferably a bi-phenyl group. Most preferably R⁷ is a 3-phenyl-phenyl group or a 2-phenyl-phenyl group. The phenyl groups are preferably either unsubstituted or substituted with an alkoxy (preferably methoxy), halo (preferably chloro), C₁₋₄ alkyl (preferably methyl or iso-propyl) or hydroxy. It is preferred that the substituent is on the distal phenyl ring, preferably at the 2-position.

### Triazoles

It is preferred that the compounds are of formula (IVb).

R¹⁰ is preferably selected from H, and C₁₋₄ alkyl (e.g. methyl, iso-propyl) and more preferably C₁₋₄ alkyl. In some embodiments the group may be unsubstituted, but when the group is substituted, preferred substituent groups include halo, hydroxy, and amino. Most preferably, R¹⁰ is methyl.

In some embodiments it is preferred that both R^{N13} and R^{N14} are substituted, and in other embodiments that only one or neither of R^{N9} and R^{N10} are substituted. Each of R^{N13} and R^{N14} are preferably independently selected from H, R, R′, where R and R′ are as defined above, and more preferably selected from H and R. R is preferably an optionally substituted C₁₋₄ alkyl group. The preferred substituents for R and R′ include halo, hydroxy, amino and acetyl.

R⁹ is preferably an optionally substituted bi-C₆ aryl group and is more preferably a bi-phenyl group. Most preferably R⁹ is a 3-phenyl-phenyl group. The phenyl groups are preferably either unsubstituted or substituted with an alkoxy (preferably methoxy), halo (preferably chloro), C₁₋₄ alkyl (preferably methyl or iso-propyl) or hydroxy. It is preferred that the substituent is on the distal phenyl ring, preferably at the 2-position.

The selectivity of the compound for antagonizing 5-HT_{2B} receptors over 5-HT_{2A} and/or 5-HT_{2C} receptors can be quantified by dividing the Ki for 5-HT_{2B} (see below) by the Ki for 5-HT_{2A/2C} (see below). The resulting ratio is preferably 10 or more, more preferably 100 or more.

The following examples illustrate the invention.

### Preparative HPLC System

Preparative HPLC was carried out on a C18-reverse-phase column (10×2.1 cm i.d Genesis column with 7 μm particle size), eluting with a gradient of acetonitrile (containing 0.1 trifluoroacetic acid) in water (containing 0.1% trifluoroacetic acid) at a flow rate of 5 ml/min. UV detection at 230 nm was used unless otherwise stated.

### LC/MS Systems

The Liquid Chromatography Mass Spectroscopy (LC/MS) systems used:

### LC/MS System A:

Mass Spectrometer—Platform LC with electrospray source operating in positive and negative Ion mode. HP1100 system running at 2.0 mL/min, 200 μL/min split to the ESI source with inline HP1100 DAD detection and SEDEX ELS detection.

Mobile Phase: A) Water 0.1% Formic Acid
B) Acetonitrile 0.1% Formic Acid

### Gradient

| Time | Flow | | |
|---|---|---|---|
| (min) | (mL/min) | % A | % B |
| 0.00 | 2.0 | 95 | 5 |
| 0.50 | 2.0 | 95 | 5 |
| 4.50 | 2.0 | 5 | 95 |
| 5.00 | 2.0 | 5 | 95 |
| 5.50 | 2.0 | 95 | 5 |

### Column—Luna 3u C18(2) 30×4.6 mm

### LC/MS System B:

Mass Spectrometer—Finnigan TSQ700 with electrospray source operating in positive or negative ion mode. HP1050 system running at 2.0 mL/min, 200 μL/min split to the ESI source with inline HP1050 Single Wavelength UV detector at 254 nm.

Mobile Phase: A) Water 0.1% formic Acid
B) Acetonitrile 0.1% formic Acid

### Gradient:

| Time | Flow | | |
|---|---|---|---|
| (min) | (mL/min) | % A | % B |
| 0.00 | 2.0 | 95 | 5 |
| 1.00 | 2.0 | 95 | 5 |
| 15.00 | 2.0 | 5 | 95 |
| 17.00 | 2.0 | 5 | 95 |
| 18.00 | 2.0 | 95 | 5 |
| 20.00 | 2.0 | 95 | 5 |

### Column—Higgins Clipius C18 5 um 100×3.0 mm

¹H NMR system

The ¹H NMR spectra were recorded on a Varian Unity Inova 400, which operates at 400 MHz for ¹H. It is equipped with a 5 mm inverse detection triple resonance probe for detection of ¹H. The magnetic field is provided by a 9.4 Tesla Oxford instruments super-conducting magnet. The host computer is a Sun Microsystems SunBlade 1000 workstation.

### Microwave System

Where microwave heating is specified, the Smith Synthesizer™ was used.

### EXAMPLE 1

### Example 1(a) (i)

### Synthesis of N⁴-aryl-6-methyl-pyrimidine-2,4-diamines

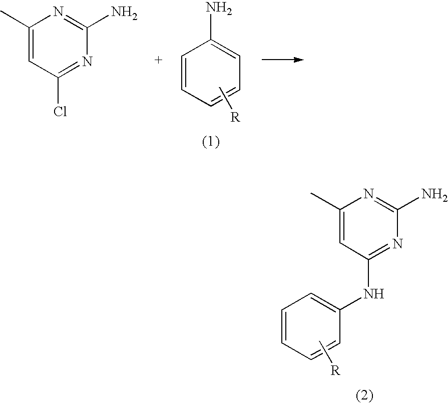 [0222] N⁴-phenyl-6-methyl-pyrimidine-2,4-diamine hydrochloric acid (2A)

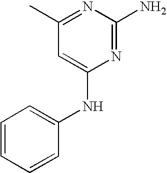

In a microwave vial (5 mL) was placed 2-amino-4-chloro-6-methylpyrimidine (143 mg), aniline (1A, 92 μL) and water (3 mL). The vessel was sealed with a crimped septum cap, and placed in the microwave cavity. The vial was heated at 165° C. for 10 minutes, after this time the vial was allowed to cool to room temperature, whereupon the title compound was purified by RP-HPLC (24 mg, 6%) as a pale pink solid. LC/MS System B: Rₜ=2.75 min, m/z (ES⁺)=201 ((M+H) for C₁₁H₁₂N₄).

### Compounds 2E-2K, 2M-2AK

Similarly, replacing aniline with other compounds of formula (1):
4-iodoaniline (Compound 1E);
4-bromoaniline (Compound 1F);
3-iodoaniline (Compound 1G);
3-(trifluoromethyl)aniline (Compound 1H);
4-fluoroaniline (Compound 1I);
5-aminoindane (Compound 1J);
4-morpholinoaniline (Compound 1K);
3,4-difluoroaniline (Compound 1M);
3,4-dichloroaniline (Compound 1N);
2-amino-4-bromophenol (Compound 1O);
3,4-dimethoxyaniline (Compound 1P);
3-aminophenol (Compound 1Q);
4-aminoindane (Compound 1R);
3-bromo-4-methylaniline (Compound 1S);
3-bromo-2-methylaniline (Compound 1T);
4-methylaniline (Compound 1U);
4′-aminoacetanilide (Compound 1V);
4-amino-benzamide (Compound 1W);
3-aminobenzylalcohol (Compound 1X);
3-chloro-4-iodoaniline (Compound 1Y);
3-amino-benzamide (Compound 1Z);
6-amino-indan-1-one (Compound 1AA);
6-aminobenzothiazole (Compound 1AB);
3-chloro-4-methoxyaniline (Compound 1AC);
3-phenoxyaniline (Compound 1AD);
4-phenoxyaniline (Compound 1AE);
3-bromoaniline (Compound 1AF);
2-iodoaniline (Compound 1AG);
2-phenoxyaniline (Compound 1AH);
4-(trifluoromethyl)aniline (Compound 1AI);
2,5-dibromoaniline (Compound 1AJ);
3-iodo-4-methylaniline (Compound 1AK);
   and following the procedures of preparation of 2A above, the following compounds of the formula (2) were prepared:
N⁴-(4-iodo-phenyl)-6-methyl-pyrimidine-2,4-diamine hydrochloride (2E)

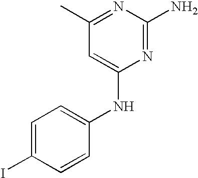

(170 mg, 47%) as an olive coloured solid. LC/MS System B: Rₜ=4.69 min, m/z (ES⁺)=327 ((M+H) for C₁₁H₁₁IN₄).
N⁴-(4-bromo-phenyl)-6-methyl-pyrimidine-2,4-diamine hydrochloride (2F)

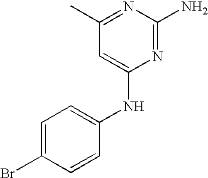

(191 mg, 61%) as a white solid. LC/MS System B: Rₜ=4.23 min, m/z (ES⁺)=279, 281 for ((M+H) for C₁₁H₁₁BrN₄).
N⁴-(3-iodo-phenyl)-6-methyl-pyrimidine-2,4-diamine hydrochloride (2G)

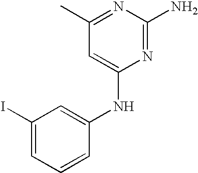

(204 mg, 56%) as a white solid. LC/MS System B: Rₜ=4.48 min, m/z (ES⁺)=327 ((M+H) for C₁₁H₁₁N₄).
6-Methyl-N⁴-(3-trifluoromethyl-phenyl)-pyrimidine-2,4-diamine, hydrochloride (2H)

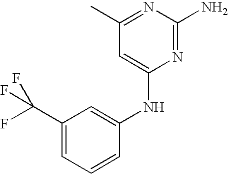

(207 mg, 68%) as a white solid. LC/MS System B: Rₜ=4.68 min, m/z (ES⁺)=269 ((M+H) for C₁₂H₁₁F₃N₄).
N⁴-(4-fluoro-phenyl)-6-methyl-pyrimidine-2,4-diamine hydrochloride (2I)

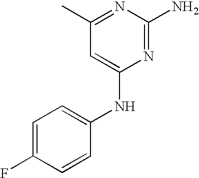

(92 mg, 36%) as a white solid. LC/MS System B: Rₜ=3.4 min, m/z (ES⁺)=219 ((M+H) for C₁₁H₁₁FN₄).
N⁴-indan-5-yl-6-methyl-pyrimidine-2,4-diamine hydrochloride (2J)

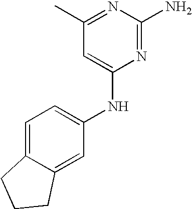

(220 mg, 79%) as a fawn coloured solid. LC/MS System B: Rₜ=4.64 min, m/z (ES⁺)=241 ((M+H) for C₁₄H₁₆N₄).
6-methyl-N⁴-(4-morpholin-4-yl-phenyl)-pyrimidine-2,4-diamine hydrochloride (2K)

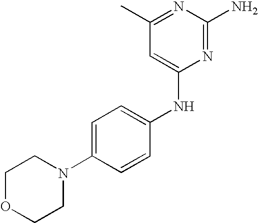

(254 mg, 79%) as a grey coloured solid. LC/MS System B: Rₜ=3.19 min, m/z (ES⁺)=286 ((M+H) for C₁₅H₁₉N₅O)
N⁴-(3,4-difluoro-phenyl)-6-methyl-pyrimidine-2,4-diamine hydrochloride (2M)

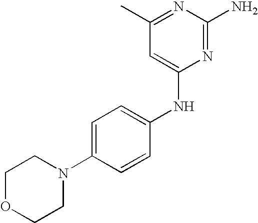

(51 mg, 19%) as a cream coloured solid. LC/MS System B: Rₜ=3.67 min, m/z (ES⁺)=237 ((M+H) for C₁₁H₁₀F₂N₄).
N⁴-(3,4-dichloro-phenyl)-6-methyl-pyrimidine-2,4-diamine hydrochloride (2N)

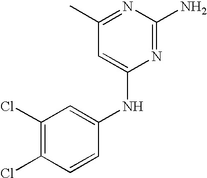

(246 mg, 80%) as a fawn-coloured solid. LC/MS System B: Rₜ=4.42 min, m/z (ES⁺)=269, 271 for ((M+H) for C₁₁H₁₀Cl₂N₄).
2-(2-amino-6-methyl-pyrimidin-4-ylamino)-4-bromo-phenol hydrochloride (20)

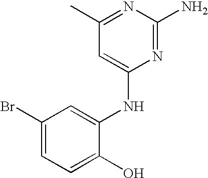

(127 mg, 38%) as a brown coloured solid. LC/MS System B: Rₜ=3.59 min, m/z (ES⁺)=295, 297 ((M+H) for C₁₁H₁₁BrN₄O).
N⁴-(3,4-dimethoxy-phenyl)-6-methyl-pyrimidine-2,4-diamine hydrochloride (2P)

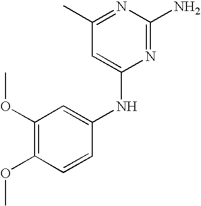

(66 mg, 22%) as a white solid. LC/MS System B: Rₜ=3.26 min, m/z (ES⁺)=261 ((M+H) for C₁₃H₁₆N₄O₂).
3-(2-amino-6-methyl-pyrimidin-4-ylamino)-phenol hydrochloride (2Q)

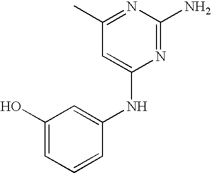

(123 mg, 49%) as a white solid. LC/MS System B: Rₜ=2.77 min, m/z (ES⁺)=217 ((M+H) for C₁₁H₁₂N₄O).
N⁴-indan-4-yl-6-methyl-pyrimidine-2,4-diamine hydrochloride (2R)

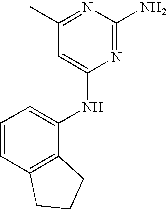

(182 mg, 66%) as a beige coloured solid. LC/MS System B: Rₜ=4.03 min, m/z (ES⁺)=241 ((M+H) for C₁₄H₁₆N₄).
N⁴-(3-bromo-4-methyl-phenyl)-6-methyl-pyrimidine-2,4-diamine hydrochloride (2S)

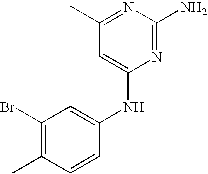

(270 mg, 82%) as a fawn coloured solid. LC/MS System B: Rₜ=4.39 min, m/z (ES⁺)=293, 295 ((M+H) for C₁₂H₁₃BrN₄).
N⁴-(3-bromo-2-methyl-phenyl)-6-methyl-pyrimidine-2,4-diamine hydrochloride (2T)

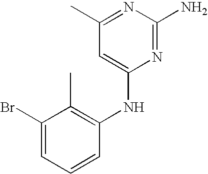

(289 mg, 88%) as a white solid. LC/MS System B: Rₜ=4.12 min, m/z (ES⁺)=293, 295 ((M+H) for C₁₂H₁₃BrN₄).
6-methyl-N⁴-p-tolyl-pyrimidine-2,4-diamine hydrochloride (2U)

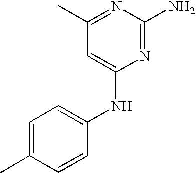

(203 mg, 81%) as pale yellow crystalline needles. LC/MS System B: Rₜ=3.71 min, m/z (ES⁺)=215 ((M+H) for C₁₂H₁₄N₄).
N-[4-(2-amino-6-methyl-pyrimidin-4-ylamino)-phenyl]-acetamide hydrochloride (2V)

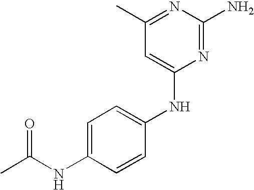

(152 mg, 52%) as a pink solid. LC/MS System B: Rₜ=2.82 min, m/z (ES⁺)=258 ((M+H) for C₁₃H₃₅N₅O).
4-(2-amino-6-methyl-pyrimidin-4-ylamino)-benzamide hydrochloride (2W)

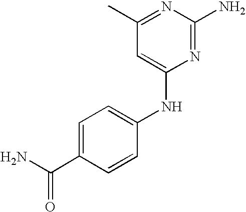

(239 mg, 85%) as a white solid. LC/MS System B: Rₜ=2.50 min, m/z (ES⁺)=244 ((M+H) for C₁₂H₁₃N₅O).
[3-(2-amino-6-methyl-pyrimidin-4-ylamino)-phenyl]-methanol hydrochloride (2×)

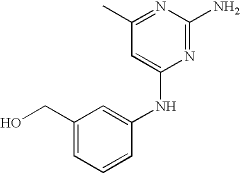

(138 mg, 52%) as a cream coloured solid. LC/MS System B: Rₜ=2.67 min, m/z (ES⁺)=231 ((M+H) for C₁₂H₁₄N₄O).
N-(3-chloro-4-iodo-phenyl)-6-methyl-pyrimidine-2,4-diamine hydrochloride (2Y)

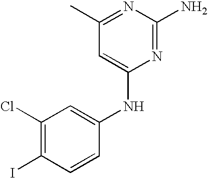

(321 mg, 81%) as a pale yellow solid. LC/MS System B: Rₜ=4.63 min, m/z (ES⁺)=361 ((M+H) for C₁₁H₁₀Cl₁N₄).
3-(2-amino-6-methyl-pyrimidin-4-ylamino)-benzamide hydrochloride (2Z)

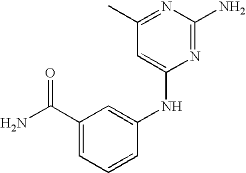

(178 mg, 64%) as a pale pink solid. LC/MS System B: Rₜ=2.46 min (weak), m/z (ES⁺)=244 ((M+H) for C₁₂H₁₃N₅O).
6-(2-amino-6-methyl-pyrimidin-4-ylamino)-indan-1-one hydrochloride (2A-A)

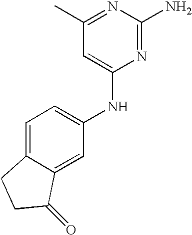

(205 mg, 71%) as a tan coloured solid. LC/MS System B: Rₜ=3.16 min, m/z (ES⁺)=255 ((M+H) for C₁₄H₁₄N₄O).
N⁴-benzothiazol-6-yl-6-methyl-pyrimidine-2,4-diamine hydrochloride (2AB)

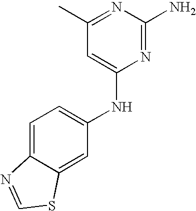

(246 mg, 84%) as a pale yellow solid. LC/MS System B: Rₜ=3.19 min, m/z (ES⁺)=258 ((M+H) for C₁₂H₁₁N₅S).
N⁴-(3-chloro-4-methoxy-phenyl)-6-methyl-pyrimidine-2,4-diamine, hydrochloride (2AC)

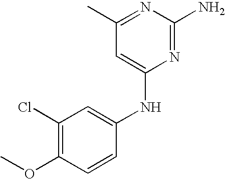

(188 mg, 62%) as a lilac solid. LC/MS System B: Rₜ=3.91 min, m/z (ES⁺)=265 ((M+H) for C₁₂H₁₃ClN₄O).
6-methyl-N⁴-(3-phenoxy-phenyl)-pyrimidine-2,4-diamine hydrochloride (2AD)

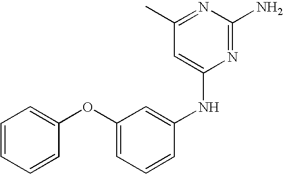

(234 mg, 71%) as a tan coloured solid. LC/MS System B: Rₜ=5.40 min, m/z (ES⁺)=293 ((M+H) for C₁₇H₃₆N₄O).
6-methyl-N⁴-(4-phenoxy-phenyl)-pyrimidine-2,4-diamine hydrochloride (2AE)

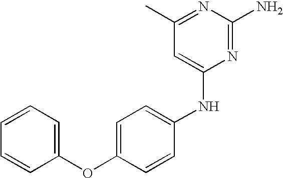

(214 mg, 65%) as a pale pink solid. LC/MS System B: Rₜ=5.57 min, m/z (ES⁺)=293 ((M+H) for C₁₇H₁₆N₄O).
N⁴-(3-bromo-phenyl)-6-methyl-pyrimidine-2,4-diamine trifluoroacetic acid (2AF)

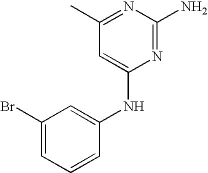

was purified by RP-HPLC to give the title compound (24 mg, 6%) as a pale pink solid. LC/MS System B: Rₜ=4.19 min, m/z (ES⁺)=279, 281 ((M+H) for C₁₁H₁₁BrN₄).
N⁴-(2-iodo-phenyl)-6-methyl-pyrimidine-2,4-diamine (2AG)

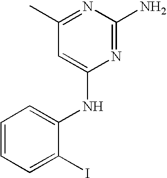

trifluoroacetic acid was purified by RP-HPLC to give the title compound (137 mg, 310) as a tan coloured solid. LC/MS System B: Rₜ=3.57 min, m/z (ES⁺)=327 ((M+H) for C₁₁H₁₁N₄).
6-methyl-N⁴-(2-phenoxy-phenyl)-pyrimidine-2,4-diamine trifluoroacetic acid (2AH)

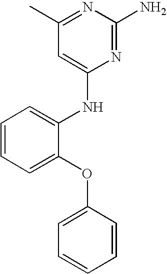

was purified by RP-HPLC to give the title compound (40 mg, 10%) as a beige coloured solid. LC/MS System B: Rₜ=4.90 min, m/z (ES⁺)=293 ((M+H) for C₁₇H₁₆N₄O).
6-methyl-N-(4-trifluoromethyl-phenyl)-pyrimidine-2,4-diamine trifluoroacetic acid (2AI)

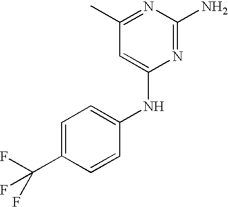

was purified by RP-HPLC to the title compound (62 mg, 16%) as a give a pale fawn coloured solid. LC/MS System B: Rₜ=4.65 min, m/z (ES⁺)=269 ((M+H) for C₁₂H₁₁F₃N₄).
N⁴-(2,5-dibromo-phenyl)-6-methyl-pyrimidine-2,4-diamine trifluoroacetic acid (2AJ)

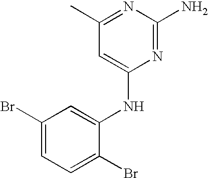

was purified by RP-HPLC to give the title compound (10 mg, 2 t) as a tan coloured solid. LC/MS System A: Rₜ=2.05 min, m/z (ES⁺)=359 ((M+H) for C₁₁H₁₀Br₂N₄).
N⁴-(3-iodo-4-methyl-phenyl)-6-methyl-pyrimidine-2,4-diamine trifluoroacetic acid (2AK)

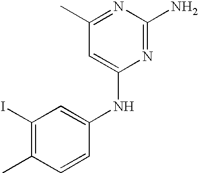

was purified by RP-HPLC to give the title compound (16 mg, 4%) as a white solid. LC/MS System A: Rₜ=2.15 min, m/z (ES⁺)=341 ((M+H) for C₁₂H₁₃N₁₄).
N⁴-(2-bromo-phenyl)-6-methyl-pyrimidine-2,4-diamine (2AM)

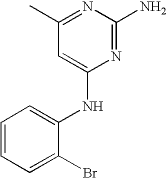

In a Microwave vial (5 mL) was placed 2-amino-4-chloro-6-methylpyrimidine (143 mg), 2-bromoaniline (Compound 1AM, 88 μL) and water (3 mL). The vessel was sealed with a crimped septum cap, and placed in the microwave cavity. The vial was heated to 165° C. for 10 minutes, after this time the vial was allowed to cool to room temperature. The reaction mixture was treated with solid sodium carbonate (106 mg) and then diluted with water (15 mL), the aqueous solution was then extracted with ethyl acetate (3×20 mL), dried over magnesium sulfate, filtered and the solvent removed under reduced pressure to give the title compound (117 mg, 42%) as a white solid. LC/MS System B: Rₜ=3.22 min, m/z (ES⁺)=279, 281 ((M+H) for C₁₁H₃₁BrN₄).

Similarly, replacing 2-bromoaniline with other compounds of formula (1):
3-fluoroaniline (Compound 1AN);
N-(4-aminophenyl)-N-methylacetamide (Compound 1AO); and
5-bromo-2-methyl-phenylamine (Compound 1AP);
   and following the procedures of preparation of Compound 2AL above, the following compounds of the formula (2) were prepared:
N⁴-(3-fluoro-phenyl)-6-methyl-pyrimidine-2,4-diamine (2AN)

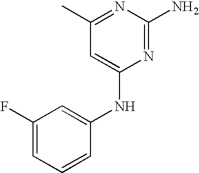

(123 mg, 56%) as a white solid. LC/MS System B: Rₜ=3.49 min, m/z (ES⁺)=219 ((M+H) for C₁₁H₁₁FN₄).
N-[4-(2-amino-6-methyl-pyrimidin-4-ylamino)-phenyl]-N-methyl-acetamide (2AO)

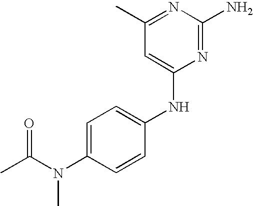

(294 mg, 94%) as a fawn coloured solid. LC/MS System B: Rₜ=2.92 min, m/z (ES⁺)=272 ((M+H) for C₁₄H₁₇N₅O).
N⁴-(5-bromo-2-methyl-phenyl)-6-methyl-pyrimidine-2,4-diamine (2AP)

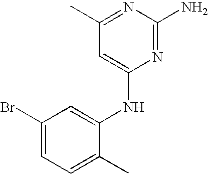

(262 mg, 89%) as a white solid. LC/MS System B: Rₜ=3.92 min, m/z (ES⁺)=293, 295 ((M+H) for C₁₂H₁₃BrN₄).

### Example 1(A)(II)

### Synthesis of N⁴-biaryl-6-methyl-pyrimidine-2,4-diamines

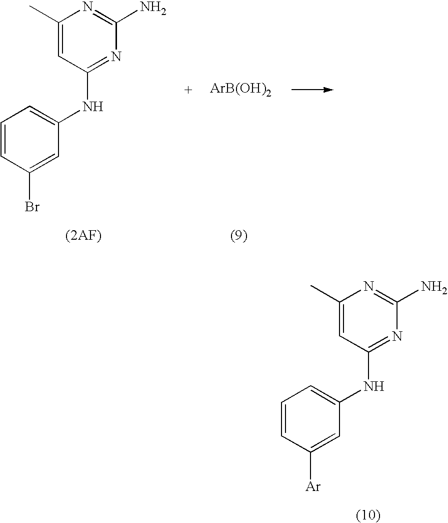 [0299] N⁴-biphenyl-3-yl-6-methyl-pyrimidine-2,4-diamine trifluoroacetic acid (10A)

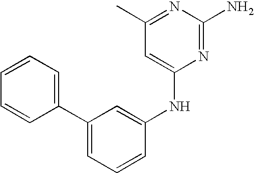

In a microwave vial (5 mL) was placed N⁴-(3-bromo-phenyl)-6-methyl-pyrimidine-2,4-diamine (2AF, 279 mg), benzeneboronic acid (9A, 122 mg), palladium (0) tetrakis(triphenylphosphine) (46 mg), 2M cesium carbonate (2 mL), and N,N-dimethylformamide (3 mL). The vial was heated to 140° C. for 3 minutes, allowed to cool to room temperature and then filtered through a short pad of hyflo to remove palladium residues. The filtrate was concentrated under reduced pressure and partitioned between water (25 mL) and ethyl acetate (25 mL), the aqueous was further extracted with ethyl acetate (2×25 mL). The combined ethyl acetate extracts were dried over magnesium sulfate, and filtered, the filtrate was concentrated under reduced pressure to afford an oil. Purification by RP-HPLC to give the title compound (185 mg, 47%) a cream solid. LC/MS System B: Rₜ=4.92 min, m/z (ES⁺)=277 ((M+H) for C₁₇H₁₆N₄).

### Compounds 10B-10J

Similarly, replacing benzeneboronic acid with other compounds of formula (9):
3,4-dimethoxybenzeneboronic acid (9B);
3-acetylbenzeneboronic acid (9C);
3-pyridylboronic acid (9D);
3-methylbenzeneboronic acid (9E);
2-methoxybenzeneboronic acid (9F);
3-hydroxybenzeneboronic acid (9G);
4-(N,N-dimethylaniline)boronic acid (9H);
3-acetamidobenzeneboronic acid (9I);
4-(methanesulphonyl)benzeneboronic acid (9J);
   and following the procedures of preparation of 10A above, the following compounds of the formula (10) were prepared:
N-(3′,4′-dimethoxy-biphenyl-3-yl)-6-methyl-pyrimidine-2,4-diamine trifluoroacetic acid (10B)

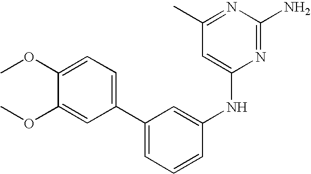

(81 mg, 45%) as a white solid. LC/MS System B: Rₜ 5.17 min, m/z (ES⁺)=337 ((M+H) for C₁₉H₂₀N₄O₂).
1-[3′-(2-amino-6-methyl-pyrimidin-4-ylamino)-biphenyl-3-yl]-ethanone trifluoroacetic acid (10C)

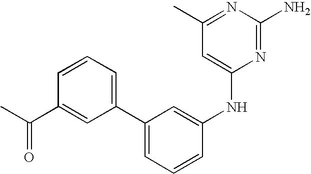

(47 mg, 27%) as a cream solid. LC/MS System B: Rₜ=5.24 min, m/z (ES⁺)=319 ((M+H) for C₁₉H₁₈N₄O).
6-methyl-N⁴-(3-pyridin-3-yl-phenyl)-pyrimidine-2,4-diamine bis trifluoroacetic acid (10D)

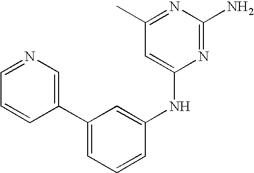

(39 mg, 20%) as a white solid. LC/MS System B: Rₜ=2.88 min, m/z (ES⁺)=278 ((M+H) for C₁₆H₁₅N₅).
6-methyl-N⁴-(3′-methyl-biphenyl-3-yl)-pyrimidine-2,4-diamine trifluoroacetic acid (10E)

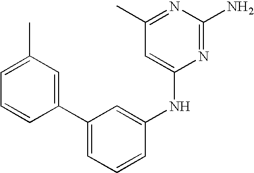

(96 mg, 59%) as a cream solid. LC/MS System B: Rₜ=5.71 min, m/z (ES⁺)=291 ((M+H) for C₁₈H₁₈N₄).
N⁴-(2′-methoxy-biphenyl-3-yl)-6-methyl-pyrimidine-2,4-diamine trifluoroacetic acid (10F)

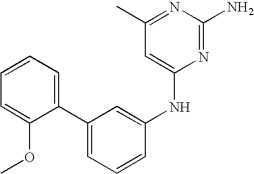

(93 mg, 55%) as a cream solid. LC/MS System B: Rₜ=5.13 min, m/z (ES⁺)=307 ((M+H) for C₁₈H₁₈N₄O).
3′-(2-amino-6-methyl-pyrimidin-4-ylamino)-biphenyl-3-ol trifluoroacetic acid (10G)

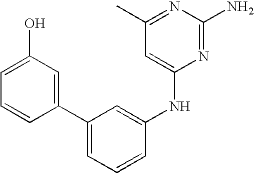

(11 mg, 7%) as a white solid. LC/MS System B: Rₜ 4.50 min, m/z (ES⁺)=293 ((M+H) for C₁₇H₁₆N₄O)
N⁴-(4′-dimethylamino-biphenyl-3-yl)-6-methyl-pyrimidine-2,4-diamine bis trifluoroacetic acid (10H)

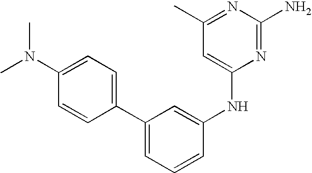

(81 mg, 37%) as a white solid. LC/MS System B: Rₜ=4.20 min, m/z (ES⁺)=320 ((M+H) for C₁₉H₂₁N₅).
N-[3′-(2-amino-6-methyl-pyrimidin-4-ylamino)-biphenyl-3-yl]-acetamide hydrochloride (10I)

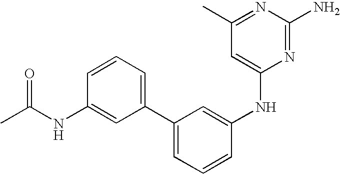

was purified by RP-HPLC, which required a small amount of concentrated hydrochloric acid to aid solubility in acetonitrile/water mixture, upon standing a solid precipitated. The solid was collected by filtration and dried to give the title compound (75 mg, 511) as a pale pink solid. LC/MS System B: Rₜ=4.83 min, m/z (ES⁺)=334 ((M+H) for C₁₉H₁₉N₅O).
N⁴-(4′-methanesulfonyl-biphenyl-3-yl)-6-methyl-pyrimidine-2,4-diamine hydrochloride (10J)

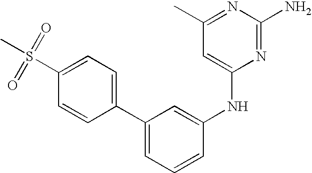

was purified by RP-HPLC, which required a small amount of concentrated hydrochloric acid to aid solubility in acetonitrile/water mixture, upon standing a solid precipitated. The solid was collected by filtration and dried to give the title compound (78 mg, 50%) as a grey solid. LC/MS System B: Rₜ=4.39 min, m/z (ES⁺)=355 ((M+H) for C₁₈H₁₈N₄O₂S).

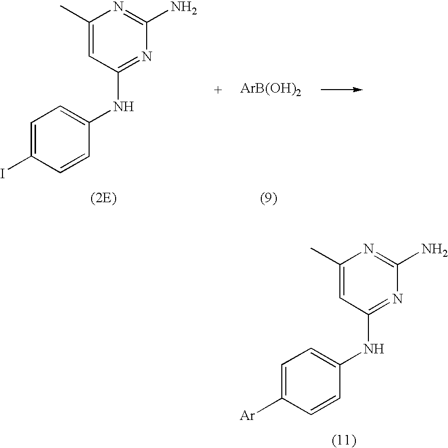

N⁴-biphenyl-4-yl-6-methyl-pyrimidine-2,4-diamine trifluoroacetic acid (11A)

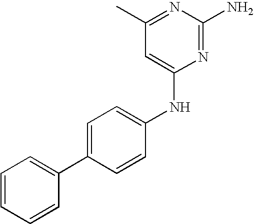

In a microwave vial (5 mL) was placed N⁴-(4-iodo-phenyl)-6-methyl-pyrimidine-2,4-diamine (2E, 362 mg), benzeneboronic acid (9A, 122 mg), palladium (0) tetrakis(triphenylphosphine) (46 mg), 2M cesium carbonate (2 mL), and N,N-dimethylformamide (3 mL). The vial was heated to 140° C. for 3 minutes, allowed to cool to room temperature and then filtered through a short pad of hyflo to remove palladium residues. The filtrate was concentrated under reduced pressure and partitioned between water (25 mL) and ethyl acetate (25 mL), the aqueous was further extracted with ethyl acetate (2×25 mL). The combined ethyl acetate extracts were dried over magnesium sulfate, and filtered, the filtrate was concentrated under reduced pressure to afford an oil. Purification by RP-HPLC gave the title compound (136 mg, 35%) as a cream solid. LC/MS System B: Rₜ=4.93 min, m/z (ES⁺)=277 ((M+H) for C₁₇H₁₆N₄).
6-methyl-N⁴-(3-methyl-biphenyl-4-yl)-pyrimidine-2,4-diamine (11B)

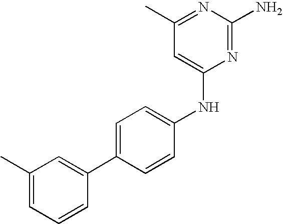

Similarly, replacing benzeneboronic acid with 3-methyl benzeneboronic acid (9E) and following the procedures of preparation of 11A above, the title compound (29.5 mg, 27%) was isolated as a pale tan coloured solid. LC/MS System B: Rₜ=5.38 min, m/z (ES⁺)=291 ((M+H) for C₁₈H₁₈N₄).
N⁴-(6,3′-dimethyl-biphenyl-3-yl)-6-methyl-pyrimidine-2,4-diamine (12)

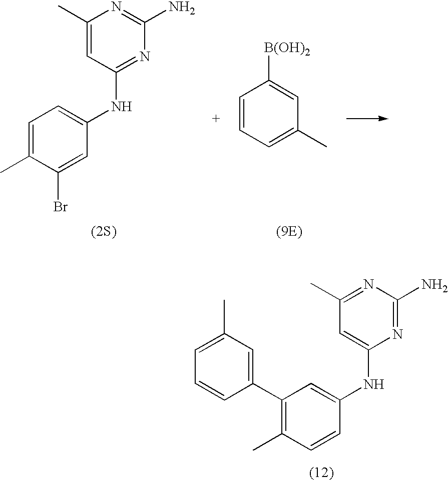

In a microwave vial (5 mL) was placed N⁴-(3-bromo-4-methyl-phenyl)-6-methyl-pyrimidine-2,4-diamine (2S, 100 mg), 3-methyl benzeneboronic acid (9E, 45 mg), palladium (0) tetrakis(triphenylphosphine) (20 mg), 2M cesium carbonate (800 μL), and N,N-dimethylformamide (3 mL). The vial was heated to 140° C. for 3 minutes, allowed to cool to room temperature and then filtered through a short pad of hyflo to remove palladium residues. The filtrate was concentrated under reduced pressure and partitioned between water (25 mL) and ethyl acetate (25 mL), the aqueous was further extracted with ethyl acetate (2×25 ml). The combined ethyl acetate extracts were dried over magnesium sulfate, and filtered, the filtrate was concentrated under reduced pressure to give crude product. The solid was purified by recrystallisation with ethyl acetate to give the title compound (15 mg, 16%) as a pink solid. LC/MS System B: Rₜ=5.43 min, m/z (ES⁺)=305 ((M+H) for C₁₉H₂₀N₄).

### Example 1(b)

### Synthesis of 4-methyl-6-aryloxy-pyrimidin-2-ylamines

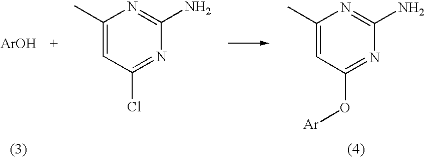 [0327] 4-methyl-6-phenoxy-pyrimidin-2-ylamine (4A)

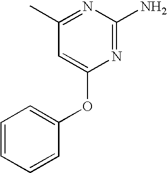

In a Microwave vial (5 mL) was placed 2-amino-4-chloro-6-methylpyrimidine (143 mg), phenol (3A, 94 mg), 2M potassium hydroxide (500 μL) and water (3.5 mL). The vessel was sealed with a crimped septum cap, and placed in the microwave cavity. The vial was heated to 165° C. for 5 minutes, after this time the vial was allowed to cool to room temperature, whereupon a solid precipitated. The solid was collected by filtration and dried in a heated desiccator to give the title compound (180 mg, 89%) as a white solid. LC/MS System B: Rₜ=3.07 min, m/z (ES⁺)=202 ((M+H) for C₁₁H₁₁N₃O).

### Compounds 4B-4E

Similarly, replacing phenol with other compounds of formula (3):
Naphthalene-1-ol (3B)
biphenyl-3-ol (3C);
biphenyl-2-ol (3D);
biphenyl-4-ol (3E);
   and following the procedures of preparation of 4A above, the following compounds of the formula (4) were prepared:
4-methyl-6-(naphthalen-1-yloxy)-pyrimidin-2-ylamine (4B)

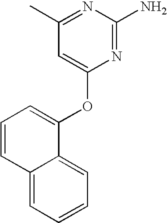

(119 mg, 33%) as a yellow solid. LC/MS System B: Rₜ=5.04 min, m/z (ES⁺)=252 ((M+H) for C₁₅H₁₃N₃O).
4-(biphenyl-3-yloxy)-6-methylpyrimidin-2-ylamine (4C)

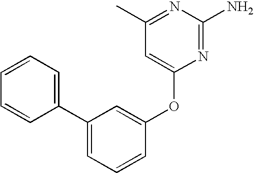

(109 mg, 28%) as a pale yellow solid. LC/MS System B: Rₜ=5.88 min, m/z (ES⁺)=278 ((M+H) for C₁₇H₁₅N₃O).
4-(biphenyl-2-yloxy)-6-methylpyrimidin-2-ylamine (4D)

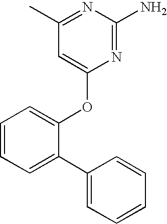

(65 mg, 17%) as a pink solid. LC/MS System B: Rₜ=5.52 min, m/z (ES⁺)=278 ((M+H) for C₁₇H₁₅N₃O).
4-(biphenyl-4-yloxy)-6-methylpyrimidin-2-ylamine (4E)

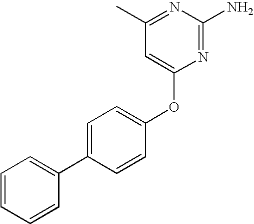

(70 mg, 25%) as a white solid. LC/MS System B: Rₜ=6.02 min, m/z (ES⁺)=278 ((M+H) for C₁₇H₁₅N₃O).

### Example 1(c)

### Synthesis of N⁴— (4-iodo-phenyl)-pyrimidin-2,4-diamine

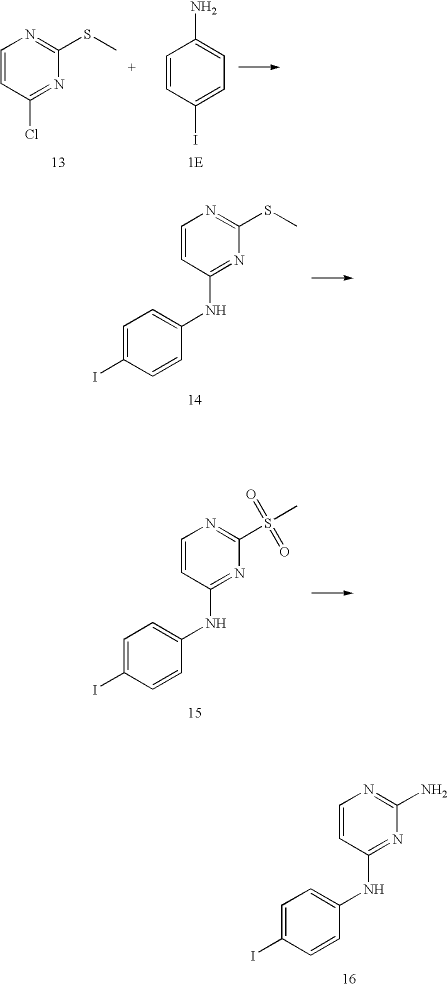 [0339] (4-Iodo-phenyl)-(2-methylsulfanyl-pyrimidin-4-yl)-amine hydrochloride (14)

In a Microwave vial (5 mL) was placed 4-chloro-2-methylsulfanyl-pyrimidine (13, 124 mg), 4-iodoaniline (Compound 1E, 177 mg) and water (2 mL). The vessel was sealed with a crimped septum cap, and placed in the microwave cavity. The vial was heated at 165° C. for 10 minutes, after this time the vial was allowed to cool to room temperature, whereupon the title compound (236 mg, 80%) precipitated from solution as a white solid. LC/MS System B: Rₜ=3.02 min, m/z (ES⁺)=344 ((M+H) for C₁₁H₁₀IN₃S).
(4-Iodo-phenyl)-(2-methanesulfonyl-pyrimidin-4-yl)-amine (15)

A mixture of (4-Iodo-phenyl)-(2-methylsulfanyl-pyrimidin-4-yl)-amine hydrochloride (14, 100 mg), meta-chloroperoxybenzoic acid (160 mg) and chloroform was stirred at room temperature for 18 hours. The mixture was diluted with chloroform (8 mL), washed with a saturated solution of sodium thiosulfate (10 mL) and a saturated solution of sodium carbonate (10 mL). The organics were dried over magnesium sulfate and the solvent removed under reduced pressure to afford the title compound (81 mg, 81%) as a peach solid. LC/MS System B: Rₜ=3.10 min, m/z (ES⁺)=374 ((M+H) for C₁₁H₁₀IN₃O₂S).
N″-(4-Iodo-phenyl)-pyrimidine-2,4-diamine (16)

In a bomb at −80° C. was added (4-Iodo-phenyl)-(2-methanesulfonyl-pyrimidin-4-yl)-amine (15, 100 mg), and liquid ammonia (10 mL). The vessel was sealed, allowed to warm to room temperature and then heated at 90° C. at 400 psi for 18 hours. The valve was opened at −80° C. and the liquid ammonia was allowed to evaporate as the bomb slowly warmed to room temperature. The residue was dissolved in ethyl acetate, washed with a saturated solution of sodium hydrogen carbonate and dried over magnesium sulfate. The solvent was removed under reduced pressure to afford the title compound (37 mg, 45%) as an orange solid. LC/MS System B: Rₜ=4.20 min, m/z (ES⁺)=313 ((M+H) for C₁₀H₉IN₄).

### EXAMPLE 2

### Example 2(a)

### Synthesis of optionally 5-substituted, 4-aryl-1H-imidazol-2-ylamines

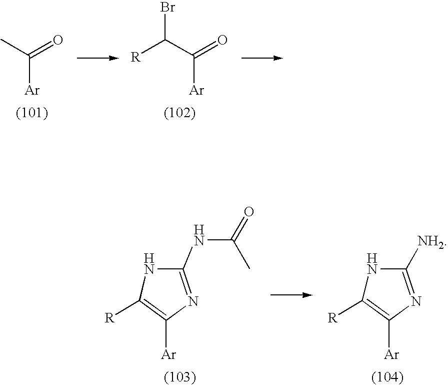 [0346] 2-Bromo-1-(2-ethoxy-naphthalen-1-yl)-ethanone (102A)

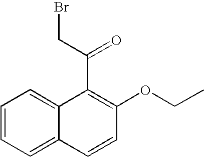

To a solution of 1-(2-ethoxy-naphthalen-1-yl)-ethanone (Compound 101A, 26 g) in tetrahydrofuran (200 mL) at 0° C. was added phenyl trimethylammonium tribromide (50 g). The mixture was stirred at 0° C. for 10 minutes and then at room temperature for 4.5 hours. The mixture was washed with water (200 mL) and the aqueous phase was extracted with diethyl ether. The combined organics were washed with water (200 mL), dried over magnesium sulfate, filtered and the solvent removed under reduced pressure to afford a dark green sticky solid. The sticky solid was triturated with diethyl ether (100 mL) and filtered to give 2,2-dibromo-1-(2-ethoxy-naphthalen-1-yl)-ethanone (12.6 g, 35%) as an off-white solid. The filtrate was evaporated to a dark green oil and purified by column chromatography, elution with 40% to 60′ dichloromethane in cyclohexane, affording 2-bromo-1-(2-ethoxy-naphthalen-1-yl)-ethanone (15.8 g, 44%) as an off-white solid. ¹H NMR (CDCl₃): 1.45 (3H, m), 4.2 (2H, m), 4.5 (2H, m), 7.2 (1H, m), 7.4 (1H, m), 7.5 (1H, m), 7.8 (2H, m), 7.9 (1H, m).

### Compounds 102B-102M

Similarly, replacing 1-(2-ethoxy-naphthalen-1-yl)-ethanone with other compounds of formula (101):
1-(4-methoxy-naphthalen-1-yl)-ethanone (101B);
1-(2-methoxy-naphthalen-1-yl)-ethanone (101C);
1-biphenyl-2-yl-ethanone (101D);
1-(1-methoxy-naphthalen-2-yl)-ethanone (101E);
1-(4-fluoro-naphthalen-1-yl)-ethanone (101F);
1-(7-bromo-naphthalen-1-yl)-ethanone (101G);
1-(5-bromo-naphthalen-1-yl)-ethanone (101H);
1-naphthalen-1-yl-propan-1-one (101I);
1-(2-methoxy-naphthalen-1-yl)-propan-1-one (101J);
3-methyl-1-naphthalen-1-yl-butan-1-one (101K);
1-benzo[b]thiophen-4-yl-ethanone (101L);
1-(2-benzyloxy-naphthalen-1-yl)-ethanone (101M);
   and following the procedures of preparation of Compound 102A above, the following compounds of the formula (102) were prepared:
2-Bromo-1-(4-methoxy-naphthalen-1-yl)-ethanone (102B)

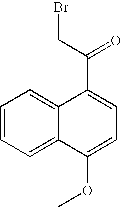

(3.0 g, 86%) as a yellow/green oil, ¹H NMR (CDCl₃): 4.05 (3H, s), 4.5 (2H, s), 6.8 (1H, d, J=8.2 Hz), 7.5 (1H, ddd, J=8.4, 7.0, 1.2 Hz), 7.6 (1H, ddd, J=8.6, 7.0, 1.5 Hz), 8.0 (1H, d, J=8.4 Hz), 8.3 (1H, d, J=8.4 Hz), 8.9 (1H, d, J=8.6 Hz).
2-Bromo-1-(2-methoxy-naphthalen-1-yl)-ethanone (102C)

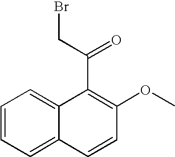

(5.25 g, 75%) as a yellow oil, ¹H NMR (DMSO-D6): 4.0 (3H, s), 4.75 (2H, s), 7.45 (1H, ddd, J=8.1, 6.7, 1.3 Hz), 7.55-7.65 (3H, m), 7.95 (1H, d, J=8.1 Hz), 8.2 (1H, d, J=9.2 Hz);
1-Biphenyl-2-yl-2-bromo-ethanone (102D)

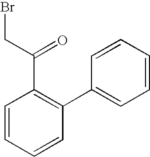

### (2.7 g, 48%), ¹H NMR (DMSO-D6): 4.4 (2H, s), 7.25-7.30 (2H, m), 7.35-7.45 (4H, m), 7.45 (1H, td, J=7.6, 1.3 Hz), 7.6 (1H, td, J=7.6, 1.4 Hz), 7.65 (1H, dd, J=7.7, 1.3 Hz).

2-Bromo-1-(1-methoxy-naphthalen-2-yl)-ethanone (102E)

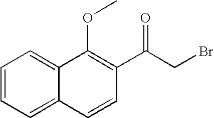

(13.2 g, 65%) as a white solid, ¹H NMR (DMSO-D6): 3.95 (3H, s), 4.95 (2H, s), 7.60-7.75 (4H, m), 7.95-7.80 (1H, m), 8.15-8.20 (1H, m).
2-Bromo-1-(4-fluoro-naphthalen-1-yl)-ethanone (102F)

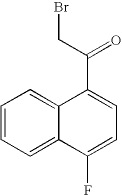

(5.7 g, 100%) as a colourless oil, ¹H NMR (CDCl₃): 4.5 (2H, s), 7.15 (1H, dd, J=9.7, 8.1 Hz), 7.55-7.70 (2H, m), 7.9-8.0 (1H, m), 8.15 (1H, m), 8.75 (1H, m).
2-Bromo-1-(7-bromo-naphthalen-1-yl)-ethanone (102G)

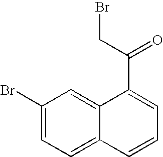

(29.7 g, 96%) as an off-white solid, ¹H NMR (CDCl₃): 4.55 (2H, s), 7.5 (1H, m), 7.6 (1H, m), 7.75 (1H, d, J=8.8 Hz), 7.95-8.0 (2H, m), 8.9 (1H, d, J=1.3 Hz).
2-Bromo-1-(5-bromo-naphthalen-1-yl)-ethanone (102H)

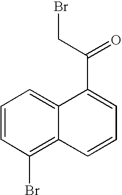

(10.9 g, 100%) as an off-white solid, ¹H NMR (CDCl₃): 4.5 (2H, s), 7.4 (1H, dd, J=8.9, 7.6 Hz), 7.6 (1H, dd, J=8.7, 7.1 Hz), 7.8-7.9 (2H, m), 8.5 (2H, m).
2-Bromo-1-naphthalen-1-yl-propan-1-one (102I)

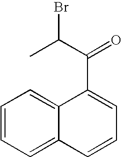

(6.0 g, 78%) as an off-white solid, ¹H NMR (CDCl₃): 1.95 (3H, d, J=6.6 Hz), 5.35 (1H, q, J=6.6 Hz), 7.45-7.60 (3H, m), 7.85-7.90 (2H, m), 8.0 (1H, d, J=8.4 Hz), 8.4 (1H, m).
2-Bromo-1-(2-methoxy-naphthalen-1-yl)-propan-1-one (102J)

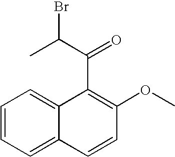

(2.87 g, 25%) as a cream solid, ¹H NMR (CDCl₃): 1.9 (3H, d, J=6.7 Hz), 3.95 (3H, s), 5.25 (1H, q, J=6.7 Hz), 7.25 (1H, d, J=9.2 Hz), 7.35 (1H, m), 7.5 (1H, m), 7.75 (2H, m), 7.9 (1H, d, J=9.0 Hz).
2-Bromo-3-methyl-1-naphthalen-1-yl-butan-1-one (102K)

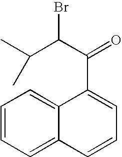

(3.9 g, 60%) as a yellow oil, ¹H NMR (CDCl₃): 1.1 (3H, d, J=6.6 Hz), 1.2 (3H, d, J=6.6 Hz), 2.5 (1H, m), 5.0 (1H, d, J=8.3 Hz), 7.5 (1H, dd, J=8.1, 7.2 Hz), 7.55 (1H, ddd, J=8.1, 6.9, 1.2 Hz), 7.6 (1H, ddd, J=8.5, 6.9, 1.5 Hz), 7.8-7.9 (2H, m), 8.0 (1H, d, J=8.3 Hz), 8.4 (1H, d, J=8.8 Hz).
1-Benzo[b]thiophen-4-yl-2-bromo-ethanone (102L)

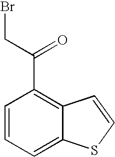

(6.8 g, 92%) as an orange oil, ¹H NMR (CDCl₃): 4.6 (2H, s), 7.4 (1H, t, J=7.8 Hz), 7.65 (1H, d, J=5.7 Hz), 7.95 (1H, dd, J=7.8, 0.9 Hz), 8.1 (1H, dt, J=7.8, 0.9 Hz), 8.3 (1H, dd, J=5.7, 0.9 Hz).
1-(2-Benzyloxy-naphthalen-1-yl)-2-bromo-ethanone (102M)

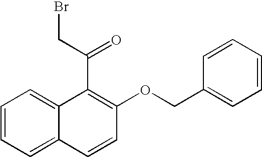

(10.2 g, 74%) as a white solid, ¹H NMR (DMSO-D6): 4.7 (2H, s), 5.35 (2H, s), 7.3-7.6 (9H, m), 7.9 (1H, d, J=8.1 Hz), 8.05 (1H, d, J=9.0 Hz).
N-[4-(2-Ethoxy-naphthalen-1-yl)-1H-imidazol-2-yl]-acetamide (103A)

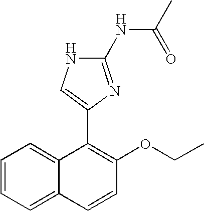

A solution of 2-bromo-1-(2-ethoxy-naphthalen-1-yl)-ethanone (Compound 102A, 4.0 g), 1-acetylguanidine (4.1 g) and N,N-dimethylformamide (24 mL) was split equally between 8 microwave vials. These vials were heated at 180° C. and treated with microwave irradiation for 180 seconds. The contents from each of the vials were combined in a round-bottomed flask and the N,N-dimethylformamide was removed under reduced pressure. The brown residue precipitated from a mixture of ethyl acetate (30 mL) and water (30 mL) to afford N-[4-(2-ethoxy-naphthalen-1-yl)-1H-imidazol-2-yl]-acetamide (1.4 g, 35%) as a cream solid. ¹H NMR (DMSO-D6) 1.25 (3H, m), 2.05 (3H, s), 4.1 (2H, m), 6.95 (1H, m), 7.3-8.3 (6H, m), 11.2-11.6 (2H, m). Mass Spectrum (m/z): 296 (M+H)⁺.

### Compounds 103B-103P

Similarly, replacing 2-bromo-1-(2-ethoxy-naphthalen-1-yl)-ethanone with other compounds of the formula (102):
2-bromo-1-(4-methoxy-naphthalen-1-yl)-ethanone (102B);
2-bromo-1-(2-methoxy-naphthalen-1-yl)-ethanone (102C);
1-biphenyl-2-yl-2-bromo-ethanone (102D);
2-bromo-1-(1-methoxy-naphthalen-2-yl)-ethanone (102E);
2-bromo-1-(4-fluoro-naphthalen-1-yl)-ethanone (102F);
2-bromo-1-(7-bromo-naphthalen-1-yl)-ethanone (102G);
2-bromo-1-(5-bromo-naphthalen-1-yl)-ethanone (102H);
2-bromo-1-naphthalen-1-yl-propan-1-one (102I);
2-bromo-1-(2-methoxy-naphthalen-1-yl)-propan-1-one (102J)
2-bromo-3-methyl-1-naphthalen-1-yl-butan-1-one (102K);
1-benzo[b]thiophen-4-yl-2-bromo-ethanone (102L);
1-(2-benzyloxy-naphthalen-1-yl)-2-bromo-ethanone (102M);
2-bromo-1-naphthalen-1-yl-ethanone (102N)

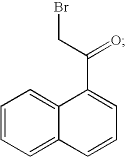

2-bromo-1-(3-methyl-benzo[b]thiophen-2-yl)-ethanone 1020)

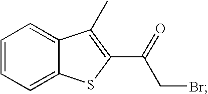

1-biphenyl-4-yl-2-bromo-ethanone (102P)

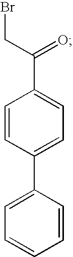

and following the procedures of preparation of Compound 103A above, the following compounds of the formula (103) were prepared:
N-[4-(4-Methoxy-naphthalen-1-yl)-1H-imidazol-2-yl]-acetamide (103B)

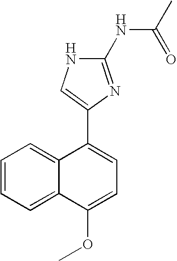

was purified by column chromatography, eluting with 5% methanol in dichloromethane, affording a pale brown solid. Recrystallisation from industrial methylated spirits gave the title compound (0.25 g, 29%) as a beige solid. ¹H NMR (DMSO-D6): 2.05 (3H, s), 3.9 (3H, s), 6.95-7.00 (2H, m), 7.45-7.55 (3H, m), 8.15 (1H, m), 8.65 (1H, m), 11.2 (1H, br s), 11.6 (1H, br s). Mass Spectrum (m/z): 282 (M+H)⁺.
N-[4-(2-Methoxy-naphthalen-1-yl)-1H-imidazol-2-yl]-acetamide (103C)

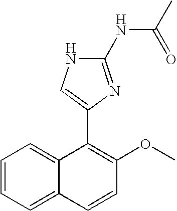

was purified by column chromatography, eluting with 1 to 2% methanol in dichloromethane, affording a peach solid. Recrystallisation from a mixture of ethanol and chloroform gave the title compound (42 mg, 16%). ¹H NMR (DMSO-D6): 2.05 (3H, s), 3.95 (3H, s), 6.95-7.00 (2H, s), 7.45-7.50 (2H, m), 7.55 (1H, d, J=7.55 Hz), 8.15 (1H, m), 8.6 (1H, d, J=7.5 Hz), 11.2 (1H, br s), 11.6 (1H, br s). Mass Spectrum (m/z): 282 (M+H)⁺.
N-(4-Biphenyl-2-yl-1H-imidazol-2-yl)-acetamide (103D)

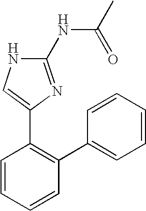

was purified by column chromatography, eluting with 70% ethyl acetate in cyclohexane, affording the title compound (171 mg, 43%) as a cream crystalline solid. ¹H NMR

(CDCl₃): 1.9 (3H, s), 6.4 (1H, br s), 7.25-7.75 (9H, m), 10.4 (1H, br s), 10.6 (1H, br s). Mass Spectrum (m/z): 282 (M+H)⁺.
N-[4-(1-Methoxy-naphthalen-2-yl)-1H-imidazol-2-yl]-acetamide (103E)

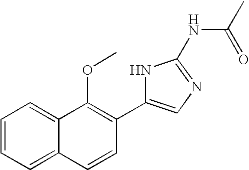

was purified by column chromatography affording an orange solid. Recrystallisation from ethanol gave the title compound (100 mg, 18%) as a white solid. ¹H NMR (DMSO-D6) 2.05 (3H, s), 3.75 (3H, s), 7.35 (1H, s), 7.4 (1H, t, J=7.2 Hz), 7.5 (1H, t, J=7.5 Hz), 7.65 (1H, d, J=8.6 Hz), 7.85 (1H, d, J=8.2 Hz), 8.0 (1H, d, J=8.6 Hz), 8.1 (1H, d, J=8.2 Hz), 11.2 (1H, br s), 11.7 (1H, br s). Mass Spectrum (m/z): 282 (M+H)⁺.
N-[4-(4-Fluoro-naphthalen-1-yl)-1H-imidazol-2-yl]-acetamide (103F)

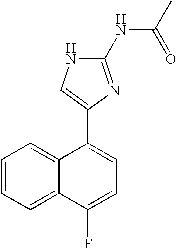

was purified by column chromatography affording a white solid. Recrystallisation from a mixture of hexane and acetone gave the title compound (632 mg, 63%) as a white solid. ¹H NMR (CDCl₃): 2.15 (3H, s), 7.0 (1H, s), 7.15-7.20 (1H, m), 7.45-7.55 (3H, m), 8.15 (1H, m), 8.4 (1H, m), 11.05 (1H, br s), 12.55 (1H, br s). Mass Spectrum (m/z): 270 (M+H)⁺.
N-[4-(7-Bromo-naphthalen-1-yl)-1H-imidazol-2-yl]-acetamide (103G)

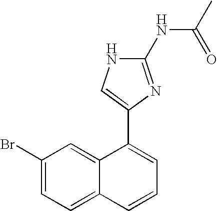

was purified by column chromatography affording the title compound (0.78 g, 26%) as a green solid. ¹H NMR (DMSO-D6) 1.95 (3H, s), 7.15 (1H, d, J=1.5 Hz), 7.5 (1H, d, J=8.1, 7.2 Hz), 7.6 (1H, dd, J=8.8, 2.2 Hz), 7.65 (1H, dd, J=7.2, 1.1 Hz), 7.8 (1H, d, J=8.1 Hz), 7.85 (1H, d, J=8.6 Hz), 9.05 (1H, d, J=2.0 Hz), 11.3 (1H, br s), 11.8 (1H, br s). Mass Spectrum (m/z): 230 (M+H)⁺.
N-[4-(5-Bromo-naphthalen-1-yl)-1H-imidazol-2-yl]-acetamide (103H)

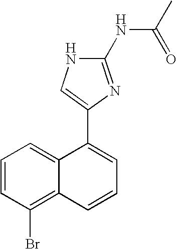

was purified by column chromatography affording a pale brown solid. Recrystallisation from acetone to gave the title compound (150 mg, 15%) as a cream solid. ¹H NMR (DMSO-D6): 2.05 (3H, s), 7.15 (1H, s), 7.4 (1H, m), 7.6 (1H, m), 7.75 (1H, d, J=7.0 Hz), 7.85 (1H, d, J=7.2 Hz), 8.05 (1H, d, J=8.3 Hz), 8.6 (1H, d, J=8.6 Hz), 11.3 (1H, br s), 11.8 (1H, br s). Mass Spectrum (m/z): 330/332 (M+H)⁺.
N-(5-Methyl-4-naphthalen-1-yl-1H-imidazol-2-yl)-acetamide (103I)

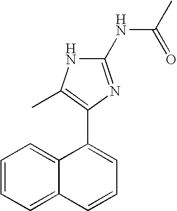

was purified by column chromatography, eluting with 1 to 2% methanol in dichloromethane, affording the title compound (120 mg, 24%) as a cream solid. ¹H NMR (DMSO-D6): 2.05 (3H, s), 2.1 (3H, s), 7.35-7.50 (4H, m), 7.8-7.9 (2H, m), 8.25 (1H, d, J=7.0 Hz), 11.05 (1H, br s), 11.55 (1H, br s). Mass Spectrum (m/z): 266 (M+H)⁺.
N-[4-(2-Methoxy-naphthalen-1-yl)-5-methyl-1H-imidazol-2-yl]-acetamide (103J)

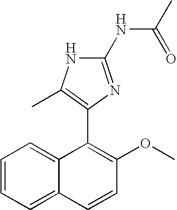

was purified by column chromatography, eluting with 50 and 75% ethyl acetate in dichloromethane, affording the title compound (210 mg, 7%) as an orange solid. ¹H NMR (DMSO-D6): 1.85 (3H, s), 2.0 (3H, s), 3.8 (3H, s), 7.3-7.6 (3H, m), 7.8-8.0 (2H, m), 11.0 (1H, br s), 11.4 (1H, br s). Mass Spectrum (m/z): 296 (M+H)⁺.
N-(5-Isopropyl-4-naphthalen-1-yl-1H-imidazol-2-yl)-acetamide (103K)

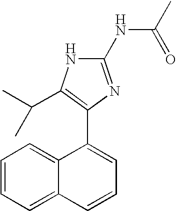

was purified by column chromatography, eluting with 30, 40 and 75% ethyl acetate in cyclohexane, affording the title compound (196 mg, 6%) as a fawn solid. ¹H NMR (DMSO-D6): 1.1 (6H, m), 2.0 (4H, m), 7.35 (4H, m), 7.85-8.05 (3H, m), 11.0-11.3 (2H, m). Mass Spectrum (m/z): 294 (M+H)⁺.
N-(4-Benzo[b]thiophen-4-yl-1H-imidazol-2-yl)-acetamide (103L)

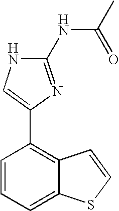

was purified by trituration with diethyl ether affording the title compound (131 mg, 51%) as a green solid. ¹H NMR (DMSO-D6): 2.05 (3H, s), 7.25 (1H, s), 7.3 (1H, t, J=7.8 Hz), 7.65 (1H, dd, J=7.5, 0.7 Hz), 7.7 (1H, d, J=5.5 Hz), 7.8 (1H, d, J=7.9 Hz), 8.15 (1H, d, J=5.5 Hz), 11.2 (1H, br s), 11.7 (1H, br s). Mass Spectrum (m/z): 258 (M+H)⁺.
N-[4-(2-Benzyloxy-naphthalen-1-yl)-1H-imidazol-2-yl]-acetamide (103M)

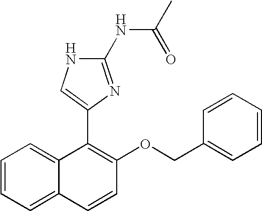

was purified by column chromatography, eluting with 50% ethyl acetate in cyclohexane, affording the title compound (100 mg, 28%) as a pale orange foam. ¹H NMR (DMSO-D6): 2.05 (3H, s), 5.2 (2H, br s), 7.0 (1H, s), 7.25-7.50 (9H, m), 7.75-8.00 (2H, m), 8.3 (1H, d, J=7.9 Hz).
N-(4-Naphthalen-1-yl-1H-imidazol-2-yl)-acetamide (103N)

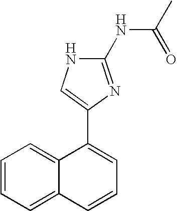

was purified by column chromatography, eluting with 5 and 10% methanol in dichloromethane, affording a dark purple solid. Recrystallisation from ethanol gave the title compound (0.95 g, 22%) as a purple solid. ¹H NMR (DMSO-D6): 2.1 (3H, s), 7.15 (1H, d, J=1.8 Hz), 7.50-7.55 (3H, m), 7.7 (1H, m), 7.8 (1H, d, J=8.0 Hz), 7.9 (1H, m), 8.75 (1H, m), 11.3 (1H, br s), 11.8 (1H, br s). Mass Spectrum (m/z): 252 (M+H)⁺.
N-[4-(3-Methyl-benzo[b]thiophen-2-yl)-1H-imidazol-2-yl]-acetamide (1030)

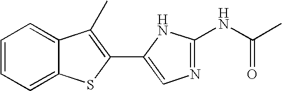

was purified by trituration from dichloromethane affording the title compound (350 mg, 35%). ¹H NMR (DMSO-D6): 2.05 (3H, s), 2.45 (3H, s), 7.05 (1H, s), 7.25 (1H, m), 7.3 (1H, m), 7.7 (1H, d, J=7.9 Hz), 7.8 (1H, d, J=7.7 Hz), 11.35 (1H, br s), 11.85 (1H, br s). Mass Spectrum (m/z): 272 (M+H)⁺.
N-(4-Biphenyl-4-yl-1H-imidazol-2-yl)-acetamide (103P)

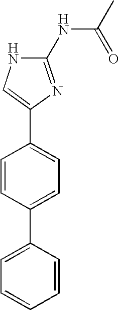

was purified by filtration of the reaction mixture affording the title compound as (2.15 g, 53%) a yellow solid. ¹H NMR (DMSO-DG): 2.05 (3H, s), 7.25-7.30 (2H, m), 7.4 (2H, m), 7.55-7.65 (4H, m), 7.75 (2H, m), 11.2 (1H, br s), 11.6 (1H, br s). Mass Spectrum (m/z): 278 (M+H)⁺.
N-[4-(2-hydroxy-naphthalen-1-yl)-1H-imidazol-2-yl]-acetamide (103Q)

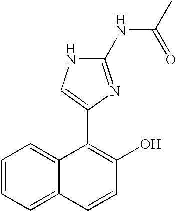

N-[4-(2-Benzyloxy-naphthalen-1-yl)-1H-imidazol-2-yl]-acetamide (Compound 103M, 0.9 g) was dissolved in ethanol (100 mL) and then palladium, 10′ on carbon (250 mg) was added. The mixture was stirred under 1 atmosphere of hydrogen for 48 hours. The mixture was filtered through a pad of hyflo and washed with industrial methylated spirits. The filtrate was concentrated under reduced pressure and the residue was purified by column chromatography to afford N-[4-(2-hydroxy-naphthalen-1-yl)-1H-imidazol-2-yl]-acetamide (300 mg, 44%). ¹H NMR (DMSO-D6): 2.1 (3H, s), 7.1 (1H, d, J=8.8 Hz), 7.25 (2H, m), 7.45 (1H, t, J=7.7 Hz), 7.65 (1H, d, J=9.0 Hz), 7.8 (1H, dd, J=8.0, 1.2 Hz), 8.25 (1H, d, J=8.1 Hz). Mass Spectrum (m/z): 268 (M+H)⁺.
4-(2-Ethoxy-naphthalen-1-yl)-1H-imidazol-2-ylamine (104A)

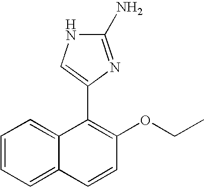

A solution of N-[4-(2-ethoxy-naphthalen-1-yl)-1H-imidazol-2-yl]-acetamide (Compound 103A, 1.4 g), industrial methylated spirits (50 mL), water (10 mL) and concentrated sulfuric acid (1 mL) was heated at 80° C. for 9 hours. After cooling to room temperature, the mixture was basified with a 1′ solution of potassium hydroxide in methanol (200 mL). The solvent was removed under reduced pressure and the residue was partitioned between ethyl acetate and water. The organic phase was washed with water (40 mL), dried over magnesium sulfate, filtered and the solvent removed under reduced pressure to afford 4-(2-ethoxy-naphthalen-1-yl)-1H-imidazol-2-ylamine (0.57 g, 47%) as a brown solid. ¹H NMR (DMSO-D6): 1.25 (3H, t, J=6.9 Hz), 4.1 (2H, q, J=6.9 Hz), 6.65 (1H, s), 7.25-7.40 (3H, m), 7.75-7.80 (2H, m), 8.25 (1H, m). Mass Spectrum (m/z): 254 (M+H)⁺.

### Compounds 104B-104F, 104I and 104N

Similarly, replacing N-[4-(2-ethoxy-naphthalen-1-yl)-1H-imidazol-2-yl]-acetamide with other compounds of the formula (103):
N-[4-(4-methoxy-naphthalen-1-yl)-1H-imidazol-2-yl]-acetamide (103B);
N-[4-(2-methoxy-naphthalen-1-yl)-1H-imidazol-2-yl]-acetamide (103C);
4-biphenyl-2-yl-1H-imidazol-2-ylamine (103D);
N-[4-(1-methoxy-naphthalen-2-yl)-1H-imidazol-2-yl]-acetamide (103E);
N-[4-(4-fluoro-naphthalen-1-yl)-1H-imidazol-2-yl]-acetamide (103F);
N-(5-methyl-4-naphthalen-1-yl-1H-imidazol-2-yl)-acetamide (103I);
N-(4-naphthalen-1-yl-1H-imidazol-2-yl)-acetamide (103N);

and following the procedures of preparation of Compound 104A above, the following compounds of the formula (104) were prepared:
4-(4-Methoxy-naphthalen-1-yl)-1H-imidazol-2-ylamine (104B)

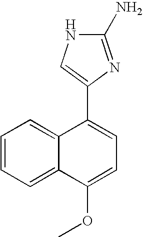

was purified by column chromatography, eluting with 10 to 50% methanol in dichloromethane, affording the title compound (17 mg, 100) as a purple solid. ¹H NMR (CDCl₃): 3.85 (3H, s), 6.4 (1H, s), 7.2-7.4 (4H, m), 7.95 (1H, m), 8.2 (1H, m). Mass Spectrum (m/z): 240 (M+H)⁺.
4-(2-Methoxy-naphthalen-1-yl)-1H-imidazol-2-ylamine (104C)

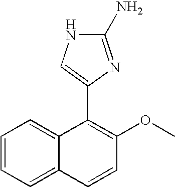

(20 mg, 83%) as a purple/brown solid. ¹H NMR (CDCl₃): 3.85 (3H, s), 6.8 (1H, s), 7.25-7.35 (2H, m), 7.4 (1H, m), 7.75 (2H, m), 8.55 (1H, d, J=8.6 Hz). Mass Spectrum (m/z): 240 (M+H)⁺.
2-Biphenyl-2-yl-1H-imidazol-4-ylamine (104D)

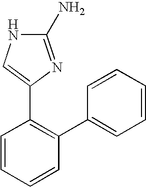

was purified by column chromatography, eluting with 10% industrial methylated spirits in cyclohexane, affording the title compound (51 mg, 44%) ¹H NMR (CDCl₃): 6.0 (1H, s) 7.2-7.3 (8H, m), 7.55 (1H, m). Mass Spectrum (m/z): 236 (M+H)⁺.
4-(1-Methoxy-naphthalen-2-yl)-1H-imidazol-2-ylamine (104E)

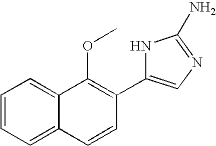

(30 mg, 35%) as a brown solid. ¹H NMR (DMSO-D6): 3.75 (3H, s), 7.1 (1H, s), 7.35 (1H, m), 7.5 (2H, m), 7.6 (1H, d, J=8.6 Hz), 7.8 (1H, d, J=7.9 Hz), 8.0 (1H, d, J=8.3 Hz). Mass Spectrum (m/z): 240 (M+H)⁺.
4-(4-Fluoro-naphthalen-1-yl)-1H-imidazol-2-ylamine (104F)

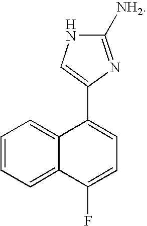

was purified by column chromatography, eluting with 10% triethylamine in ethanol, affording the title compound (80 mg, 19%) as a yellow/brown solid. ¹H NMR (DMSO-D6): 7.15 (1H, s), 7.4-7.5 (2H, m), 7.55 (1H, m), 7.65 (1H, m), 8.0-8.1 (2H, m). Mass Spectrum (m/z): 228 (M+H)⁺.
5-Methyl-4-naphthalen-1-yl-1H-imidazol-2-ylamine (104I)

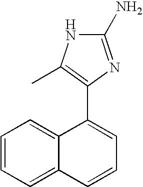

was purified by column chromatography, eluting with 10 methanol in dichloromethane, affording a yellow solid. Recrystallisation from a mixture a acetone and cyclohexane gave the title compound (3.5 mg, 7%) as a yellow solid. ¹H NMR (CDCl₃): 2.0 (3H, s), 7.35-7.45 (4H, m), 7.75 (1H, m), 7.8 (1H, m), 7.95 (1H, m). Mass Spectrum (m/z): 224 (M+H)⁺.
4-Naphthalen-1-yl-1H-imidazol-2-ylamine (104N)

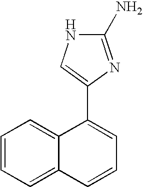

(0.62 g, 91%) as a pink solid. ¹H NMR (DMSO-D6): 5.35 (2H, br s), 6.9 (1H, s), 7.45-7.50 (3H, m), 7.6 (1H, d, J=6.6 Hz), 7.7 (1H, d, J=8.2 Hz), 7.85-7.90 (1H, m), 8.75 (1H, br s). Mass Spectrum (m/z): 210 (M+H)⁺.
4-(7-Bromo-naphthalen-1-yl)-1H-imidazol-2-ylamine (104G)

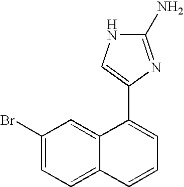

A mixture of N-[4-(7-bromo-naphthalen-1-yl)-1H-imidazol-2-yl]-acetamide (Compound 103G, 0.75 g) and concentrated hydrochloric acid (40 mL) were heated at reflux for 1 hour. On cooling to room temperature a precipitate formed, which was filtered and washed with diethyl ether affording 4-(7-bromo-naphthalen-1-yl)-1H-imidazol-2-ylamine (0.45 g, 61%) as an off white hydrochloric salt. ¹H NMR (DMSO-D6): 7.2 (1H, s), 7.5 (2H, br s), 7.60-7.65 (2H, m), 7.7 (1H, dd, J=8.8, 2.0 Hz), 7.95 (1H, d, J=8.8 Hz), 8.0 (1H, m), 8.15 (1H, d, J=2.0 Hz). Mass Spectrum (m/z): 288/290 (M+H)⁺.

### Compound 104H, 104J, 104K, 104O and 104P

Similarly, replacing N-[4-(7-bromo-naphthalen-1-yl)-1H-imidazol-2-yl]-acetamide with other compounds of the formula (103):
N-[4-(5-bromo-naphthalen-1-yl)-1H-imidazol-2-yl]-acetamide (103H);
N-[4-(2-methoxy-naphthalen-1-yl)-5-methyl-1H-imidazol-2-yl]-acetamide (103J);
N-(5-isopropyl-4-naphthalen-1-yl-1H-imidazol-2-yl)-acetamide (103K);
N-[4-(3-methyl-benzo[b]thiophen-2-yl)-1H-imidazol-2-yl]-acetamide (103O);
N-(4-biphenyl-4-yl-1H-imidazol-2-yl)-acetamide (103P);
   and following the procedures of preparation of Compound 104G above, the following compounds of the formula (104) were prepared:
4-(5-Bromo-naphthalen-1-yl)-1H-imidazol-2-ylamine (104H)

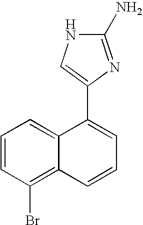

(85 mg, 72′) ¹H NMR (DMSO-D6): 7.2 (1H, s), 7.5 (1H, dd, J=8.6, 7.5 Hz), 7.55 (2H, br s), 7.65-7.75 (2H, m), 7.95 (1H, dd, J=7.5, 0.9 Hz), 8.05 (1H, d, J=8.6 Hz), 8.2 (1H, d, J=8.3 Hz). Mass Spectrum (m/z): 288/290 (M+H)⁺.
4-(2-Methoxy-naphthalen-1-yl)-5-methyl-1H-imidazol-2-ylamine (104J)

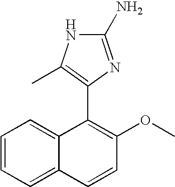

was purified by HPLC, eluting on a gradient of 20 to 80% acetonitrile in water, using ammonium acetate as buffer, affording the title compound (17 mg, 8%) as an off-white acetate salt. ¹H NMR (DMSO-D6): 1.75 (3H, s), 3.8 (3H, s), 7.3 (1H, ddd, J=8.1, 6.8, 1.2 Hz), 7.4 (1H, ddd, J=8.5, 6.8, 1.4 Hz), 7.45 (1H, d, J=8.9 Hz), 7.6 (1H, d, J=8.3 Hz), 7.8 (1H, d, J=8.9 Hz), 7.9 (1H, d, J=8.9 Hz). Mass Spectrum (m/z): 254 (M+H)⁺.
5-Isopropyl-4-naphthalen-1-yl-1H-imidazol-2-ylamine (104K)

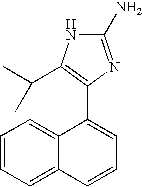

(179 mg, 90%) as a dark foam hydrochloric salt. ¹H NMR (DMSO-D6): 1.1 (6H, d, J=7.0 Hz), 2.6 (1H, m), 7.3 (2H, br s), 7.5-7.6 (5H, m), 7.7 (1H, m), 8.0 (2H, m). Mass Spectrum (m/z): 252 (M+H)⁺.
4-(3-Methyl-benzo[b]thiophen-2-yl)-1H-imidazol-2-ylamine (104O)

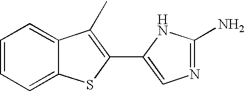

(140 mg, 83%) as a purple solid. ¹H NMR (DMSO-D6): 2.4 (3H, s), 6.85 (1H, s), 7.2 (1H, m), 7.3 (1H, m), 7.65 (1H, d, J=7.7 Hz), 7.75 (1H, d, J=7.7 Hz). Mass Spectrum (m/z) 230 (M+H)⁺.
4-Biphenyl-4-yl-1H-imidazol-2-ylamine (104P)

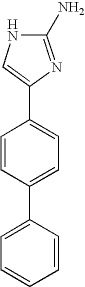

(1.6 g, 83%) as a peach hydrochloric salt. ¹H NMR (DMSO-D6): 7.35 (1H, m), 7.40-7.45 (4H, m), 7.65-7.75 (5H, m). Mass Spectrum (m/z): 236 (M+H)⁺.

### Example 2(b)

### Synthesis of 4-biphenyl-3-yl-1H-imidazol-2-ylamine

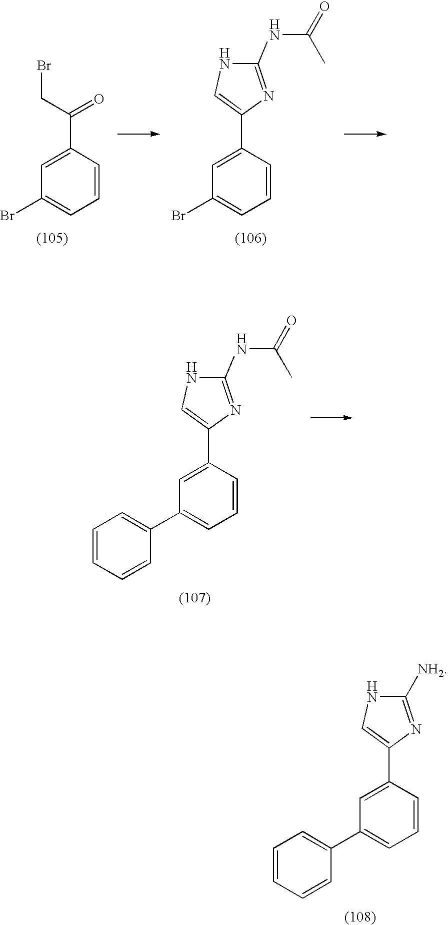 [0441] N-[4-(3-bromo-phenyl)-1H-imidazol-2-yl]-acetamide (106)

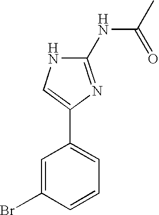

A solution of 2-bromo-1-(3-bromo-phenyl)-ethanone (105, 6.8 g), 1-acetylguanidine (7.4 g) and N,N-dimethylformamide (70 mL) was split equally between 14 microwave vials. These vials were heated at 180° C. and treated with microwave irradiation for 180 seconds. The contents from each of the vials were combined in a round-bottomed flask and the N,N-dimethylformamide was removed under reduced pressure. The brown residue was partitioned between ethyl acetate (100 mL) and water (50 mL). The organic layer was washed water (2×50 mL), dried over magnesium sulfate, filtered and the solvent removed under reduced pressure to afford an orange brown/gum. Purification by column chromatography, elution with 10 to 50% ethyl acetate in cyclohexane, afforded N-[4-(3-bromo-phenyl)-1H-imidazol-2-yl]-acetamide (106)(2.95 g, 40%) as a yellow/green solid. ¹H NMR (DMSO-D6): 1.95 (3H, S), 7.20-7.35 (3H, m), 7.65 (1H, m), 7.85 (1H, m). Mass Spectrum (m/z): 280/282 (M+H)⁺.
N-(4-biphenyl-3-yl-1H-imidazol-2-yl)-acetamide (107)

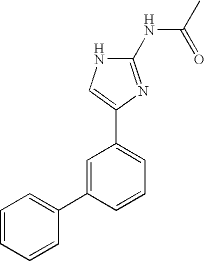

A mixture of N-[4-(3-bromo-phenyl)-1H-imidazol-2-yl]-acetamide (Compound 106, 1.0 g), aqueous solution of cesium carbonate (2M, 7.1 mL), phenylboronic acid (0.65 g), 1,4-dioxane (35 mL) and palladium (0) tetrakis(triphenylphosphine) (0.32 g) was heated at 100° C. for 30 hours. The solvent was removed under reduced pressure and the residue was partitioned between dichloromethane (40 mL) and water (40 mL). The organic phase was washed with water (40 mL), dried over magnesium sulfate, filtered and the solvent removed under reduced pressure to afford a brown solid. Purification by column chromatography, eluting with 40% ethyl acetate in dichloromethane, afforded N-(4-biphenyl-3-yl-1H-imidazol-2-yl)-acetamide (107) (0.21 g, 22%) as a fawn solid. ¹H NMR (DMSO-D6): 2.05 (3H, s), 7.30-7.45 (6H, m), 7.65 (3H, m), 7.95 (1H, m). Mass Spectrum (m/z): 278 (M+H)⁺.
4-biphenyl-3-yl-1H-imidazol-2-ylamine (108)

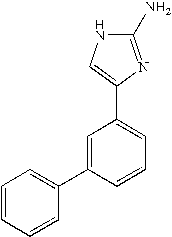

A mixture of N-(4-biphenyl-3-yl-1H-imidazol-2-yl)-acetamide

(107, 0.18 g) and concentrated hydrochloric acid (10 mL) were heated at reflux for 1 hour. The concentrated hydrochloric acid was removed under reduced pressure and the residue was purified by HPLC, eluting on a gradient of 30 to 90% acetonitrile in water, using trifluoroacetic acid as buffer afforded 4-biphenyl-3-yl-1H-imidazol-2-ylamine (108)(33 mg, 22%) as a white trifluoroacetate salt. ¹H NMR (DMSO-D6): 7.35 (1H, m), 7.45-7.50 (4H, m), 7.55-7.65 (4H, m), 7.7 (2H, m), 7.95 (1H, m). Mass Spectrum (m/z): 288/290 (M+H)⁺.

### EXAMPLE 3

### Example 3(a)

### Synthesis of 5-biphenyl-2-yl-oxazol-2-ylamine (203)

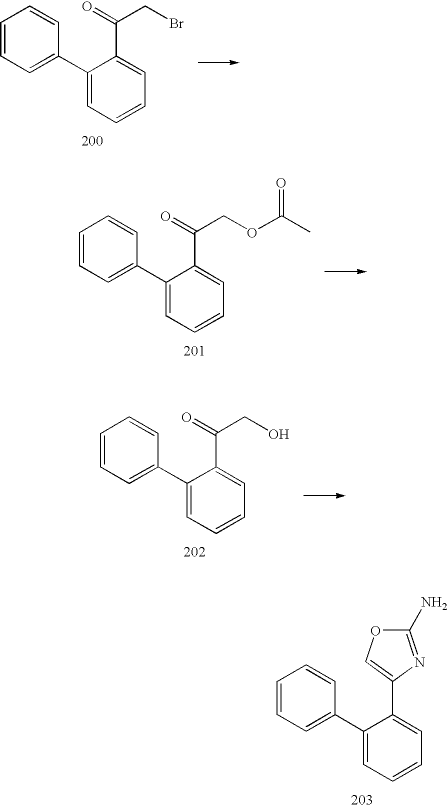 [0449] Acetic acid 2-biphenyl-2-yl-2-oxo-ethyl ester (201)

A mixture of 1-biphenyl-2-yl-2-bromo-ethanone (Compound 200, 2.9 g), N,N-dimethylformamide (60 mL) and sodium acetate (0.87 g), was heated at 90° C. for 16 hours. The N,N-dimethylformamide was removed under reduced pressure and the residue was partitioned between brine (100 mL) and ethyl acetate (100 mL). The aqueous layer was extracted with ethyl acetate (2×50 mL) and the combined organics were dried over magnesium sulfate. The solvent was removed under reduced pressure to give a dark orange gum, which was purified by column chromatography, eluting with dichloromethane, to afford acetic acid 2-biphenyl-2-yl-2-oxo-ethyl ester (2.6 g, 96%) as an orange gum. ¹H NMR (DMSO-D6) 2.0 (3H, s), 4.85 (2H, s), 7.25-7.40 (6H, m), 7.5 (1H, m), 7.6 (1H, m), 7.65 (1H, m).
1-Biphenyl-2-yl-2-hydroxy-ethanone (202)

A mixture of acetic acid 2-biphenyl-2-yl-2-oxo-ethyl ester (Compound 201, 2.6 g), industrial methylated spirits (20 mL) and 1 M hydrochloric acid (15 mL) was heated at reflux for 2 hours. The solvent was removed under reduced pressure and the crude material was partitioned between water (100 mL) and ethyl acetate (100 mL). The aqueous layer was extracted with ethyl acetate (2×50 mL) and the combined organics were washed with a saturated solution of sodium carbonate (50 mL), and dried over magnesium sulfate. The solvent was removed under reduced pressure give a pale yellow oil, which was purified by column chromatography, eluting with dichloromethane, to afford 1-biphenyl-2-yl-2-hydroxy-ethanone (0.83 g, 38%) as a colourless oil. ¹H NMR (DMSO-D6) 4.15 (2H, d, J=5.9 Hz), 5.1 (1H, t, J=5.9 Hz), 7.25-7.55 (9H, m).
5-biphenyl-2-yl-oxazol-2-ylamine (203)

In a microwave vial was placed 1-biphenyl-2-yl-2-hydroxy-ethanone (Compound 202, 0.83 g), cyanamide (0.49 g) and N,N-dimethylformamide (5 mL). The vessel was equipped with a stirrer bar, sealed with a crimped septum cap, and placed in the microwave cavity. The vial was heated at 250° C. for 10 minutes, after this time the vial was allowed to cool to room temperature, and the resultant mixture was concentrated to dryness under reduced pressure. The residue was purified by column chromatography, eluting with tert-butyl methyl ether, to afford 5-biphenyl-2-yl-oxazol-2-ylamine (0.18 g, 19%) as an orange solid. LC/MS System A: Rt=2.45 min, m/z (ES⁺)=237 ((M+H) for C₁₅H₁₂N₂₀). ¹H NMR (DMSO-D6) 6.7 (2H, br s), 7.15 (1H, m), 7.20-7.25 (3H, m), 7.35-7.40 (5H, m), 7.45 (1H, dd, J=7.9, 1.1 Hz).

### Example 3(b)

### Synthesis of 5-biphenyl-3-yl-oxazol-2-ylamines

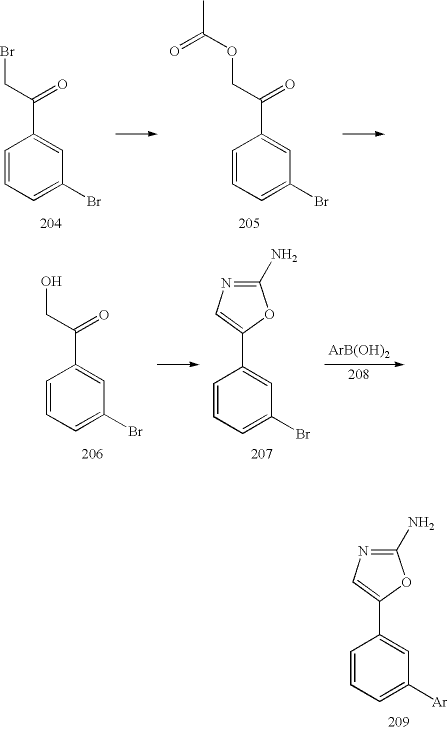 [0456] Acetic acid 2-(3-bromo-phenyl)-2-oxo-ethyl ester (205)

A mixture of 2-bromo-1-(3-bromo-phenyl)-ethanone (Compound 204, 19.1 g), sodium acetate (5.6 g) and N,N-dimethylformamide (250 mL) was heated at 90° C. for 16 hours. The N,N-dimethylformamide was removed under reduced pressure and the residue was partitioned between brine and ethyl acetate. The aqueous layer was extracted with ethyl acetate and the combined organics were dried over magnesium sulfate. The solvent was removed under reduced pressure to give acetic acid 2-(3-bromo-phenyl)-2-oxo-ethyl ester (16.9 g, 96%) as a dark orange. ¹H NMR (CDCl₃) 2.2 (3H, s), 5.25 (2H, s), 7.35 (1H, t, J=7.9 Hz), 7.7 (1H, m), 7.8 (1H, m), 8.0 (1H, m).
1-(3-Bromo-phenyl)-2-hydroxy-ethanone (206)

A mixture of acetic acid 2-(3-bromo-phenyl)-2-oxo-ethyl ester (Compound 205, 16.9 g), industrial methylated spirits (110 mL) and 1 M hydrochloric acid (85 mL) was heated at reflux for 2 hours. The solvent was removed under reduced pressure and the residue was partitioned between water (300 mL) and ethyl acetate (300 mL). The organic layer was washed with water (300 mL) and dried over magnesium sulfate. The solvent was removed under reduced pressure to afford 1-(3-bromo-phenyl)-2-hydroxy-ethanone (13.1 g, 93%) as a yellow solid. ¹H NMR (CDCl₃) 5.25 (2H, s), 7.35 (1H, t, J=7.9 Hz), 7.7 (1H, m), 7.8 (1H, m), 8.0 (1H, m).
5-(3-Bromophenyl)-oxazol-2-ylamine (207)

A solution of 1-(3-bromo-phenyl)-2-hydroxy-ethanone (Compound 206, 13.1 g), cyanamide (7.7 g), and N,N-dimethylformamide (130 mL) was split equally between 26 microwave vials. The vessels were equipped with a stirrer bar, sealed with a crimped septum cap, and placed in the microwave cavity. The vials were heated at 200° C. for 10 minutes, after this time the vials were allowed to cool to room temperature, and the resultant mixtures were combined in a round bottom flask, and concentrated under reduced pressure. The residue was partitioned between ethyl acetate (200 mL) and water (200 mL), the organic layer was washed with brine (200 mL) and the combined organics were dried over magnesium sulfate. The solvent was removed under reduced pressure to give a brown solid, which was purified by column chromatography, eluting with 50% ethyl acetate in dichloromethane, to afford 4-(3-bromophenyl)-oxazol-2-ylamine (3.57 g, 24%) as a beige solid. LC/MS System B: Rt=4.77 min, m/z (ES⁺)=239,241 ((M+H) for C₉H₇BrN₂O). ¹H NMR (DMSO-D6) 6.9 (2H, br s), 7.25-7.30 (3H, m), 7.4 (1H, m), 7.6 (1H, m).
5-Biphenyl-3-yl-oxazol-2-ylamine (209A)

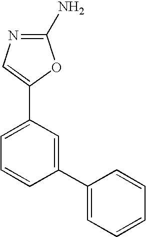

In a microwave vial was placed 4-(3-bromophenyl)-oxazol-2-ylamine (Compound 207, 200 mg), benzeneboronic acid (Compound 208A, 157 mg), palladium (0) tetrakis (triphenylphosphine) (38 mg), 2M cesium carbonate (1.65 mL), and N,N-dimethylformamide (3.0 mL). The vial was heated to 100° C. for 3 minutes, allowed to cool to room temperature and then filtered through a short pad of hyflo. The filtrate was concentrated under reduced pressure and partitioned between water (20 mL) and dichloromethane (20 mL). The organic layer was washed with water (20 mL) and dried over magnesium sulfate. The solvent was removed under reduced pressure to give an orange solid, which was recrystallised from a mixture of industrial methylated spirits and cyclohexane to afford 5-biphenyl-3-yl-oxazol-2-ylamine (70 mg, 35%) as a peach solid. LC/MS System B: Rt=6.06 min, m/z (ES⁺)=237 ((M+H) for C₁₅H₁₂N₂O). ¹H NMR (DMSO-D6) 6.8 (2H, br s), 7.25 (1H, s), 7.35 (1H, m), 7.40-7.45 (5H, m), 7.6-7.7 (3H, m).

### Compounds 209B-209M

Similarly, replacing benzeneboronic acid (208A) with other compounds of formula (208):
3-methylbenzeneboronic acid (208B);
3-hydroxybenzeneboronic acid (208C);
3-cyanobenzeneboronic acid (208D);
2-chlorobenzeneboronic acid (208E);
3-pyridylboronic acid (208F);
2-methoxybenzeneboronic acid (208G);
3-acetylbenzeneboronic acid (208H);
3-(trifluoromethyl)benzeneboronic acid (208I);
4-fluorobenzeneboronic acid (208J);
3,5-dimethylbenzeneboronic acid (208K);
4-ethylbenzeneboronic acid (208L);
3-isopropylbenzeneboronic acid (208M);
   and following the procedures of preparation of Compound 209A above, the following compounds of the formula (209) were prepared:
5-(3′-Methyl-biphenyl-3-yl)-oxazol-2-ylamine (209B)

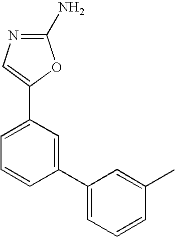

was purified by column chromatography, eluting with 20 to 40% ethyl acetate in dichloromethane, followed by recrystallisation from a mixture of ethyl acetate and cyclohexane to afford the title compound (29 mg, 12%) as a white solid, LC/MS System B: Rt=6.72 min, m/z (ES⁺)=251 ((M+H) for C₁₆H₁₄N₂O), ¹H NMR (DMSO-D6) 2.35 (3H, m), 6.8 (2H, br s), 7.15 (1H, m), 7.25 (1H, s), 7.3 (1H, t, J=7.6 Hz), 7.40-7.45 (5H, m), 7.65 (1H, m).
3′-(2-Amino-oxazol-5-yl)-biphenyl-3-ol (209C)

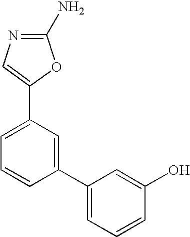

was purified by column chromatography, eluting with 20 to 40% ethyl acetate in dichloromethane, followed by recrystallisation from a mixture of ethyl acetate and cyclohexane to afford the title compound (36 mg, 14%) as an off-white solid, LC/MS System B: Rt=4.89 min, m/z (ES⁺)=253 ((M+H) for C₁₆H₁₄N₂O), ¹H NMR (DMSO-D6) 6.75 (1H, dd, J=8.1, 1.5 Hz), 6.8 (2H, br s), 7.0 (1H, m), 7.05 (1H, m), 7.20-7.25 (2H, m), 7.35-7.40 (3H, m), 7.6 (1H, m), 9.5 (1H, br s).
3-(2-Amino-oxazol-5-yl)-biphenyl-3-carbonitrile (209D)

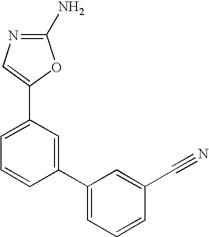

was purified by HPLC using a gradient of 20 to 80% acetonitrile in water at 1% per minute, to afford the title compound (27 mg, 7%) as an off-white solid, LC/MS System B: Rt=5.88 min, m/z (ES⁺)=262 ((M+H) for C₁₆H₁₁N₃O), ¹H NMR (DMSO-D6) 6.85 (2H, br s), 7.25-7.30 (2H, m), 7.45-7.55 (3H, m), 7.65 (1H, t, J=7.8 Hz), 7.75 (1H, m), 7.8 (1H, m), 8.0 (1H, m).
5-(2′-Chloro-biphenyl-3-yl)-oxazol-2-ylamine (209E)

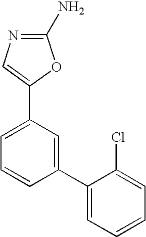

was purified by HPLC using a gradient of 20 to 80% acetonitrile in water at 10 per minute to afford the title compound (0.26 g, 7%) as a white solid, LC/MS System B: Rt=6.70 min, m/z (ES⁺)=271 ((M+H) for C₁₅H₁₁ClN₂O), ¹H NMR (DMSO-D6) 7.3 (1H, m), 7.40-7.45 (4H, m), 7.5-7.6 (6H, m).
5-(3-Pyridin-3-yl-phenyl)-oxazol-2-ylamine (209F)

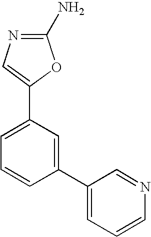

was purified by HPLC using a gradient of 20 to 80% acetonitrile in water at 1% per minute, to afford the title compound (18 mg, 5%) as a white solid, LC/MS System B: Rt=2.59 min, m/z (ES⁺)=238 ((M+H) for C₁₄H₁₁N₃O), ¹H NMR (DMSO-D6) 7.6 (2H, m), 7.8 (1H, m), 7.95-8.00 (2H, m), 8.05 (1H, s), 8.7 (1H, d, J=8.1 Hz), 8.8 (1H, d, J=4.4 Hz), 9.2-9.3 (3H, m).
5-(2′-Methoxy-biphenyl-3-yl)-oxazol-2-ylamine trifluoroacetic acid (209G)

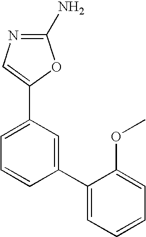

was purified by HPLC using a gradient of 20 to 80% acetonitrile in water at 1% per minute, to afford the title compound (20 mg, 5) as a white solid, LC/MS System B: Rt=6.05° min, m/z (ES⁺)=267 ((M+H) for C₁₆H₁₄N₂O₂), ¹H NMR (DMSO-D6) 3.75 (3H, s), 7.0 (1H, td, J=7.5, 1.1 Hz), 7.1 (1H, dd, J=8.3, 0.9 Hz), 7.25 (1H, dd, J=7.5, 1.8 Hz), 7.30-7.45 (4H, m), 7.6 (2H, m).
1-[3′-(2-Amino-oxazol-5-yl)-biphenyl-3-yl]-ethanone, trifluoroacetic acid (209H)

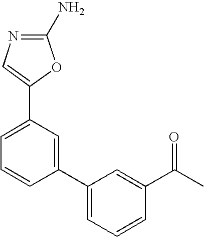

was purified by HPLC using a gradient of 20 to 80% acetonitrile in water at 1% per minute, to afford the title compound (180 mg, 5%) as a white solid, LC/MS System B: Rt=4.96 min, m/z (ES⁺)=279 ((M+H) for C₁₇H₁₄N₂O₂), ¹H NMR (DMSO-D6) 2.6 (3H, s), 7.3 (1H, m), 7.4 (1H, m), 7.55 (2H, m), 7.6-7.7 (3H, m), 7.81 (1H, m), 7.9-8.0 (2H, m), 8.15 (1H, m).
5-(3′-Trifluoromethyl-biphenyl-3-yl)-oxazol-2-ylamine, trifluoroacetic acid (209I)

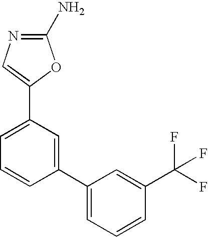

was purified by HPLC using a gradient of 20 to 80% acetonitrile in water at 1% per minute, to afford the title compound (108 mg, 32%) as a white solid, LC/MS System B: Rt=6.67 min, m/z (ES⁺)=305 ((M+H) for C₁₆H₁₁F₃N₂O), ¹H NMR (DMSO-D6) 7.50-7.75 (6H, m), 7.85 (1H, m), 7.95-8.15 (4H, m).
5-(4′-Fluoro-biphenyl-3-yl)-oxazol-2-ylamine, trifluoroacetic acid (209J)

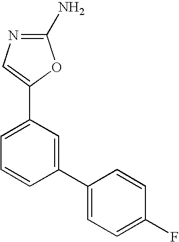

was purified by HPLC using a gradient of 20 to 80% acetonitrile in water at 1% per minute, to afford the title compound (34 mg, 2%) as a white solid, LC/MS System B: Rt=5.68 min, m/z (ES⁺)=255 ((M+H) for C₁₅H₁₁FN₂O), ¹H NMR (DMSO-D6) 7.25-7.30 (2H, m), 7.45-7.55 (3H, m), 7.65-7.75 (4H, m).
5-(3′,5′-Dimethyl-biphenyl-3-yl)-oxazol-2-ylamine, trifluoroacetic acid (209K)

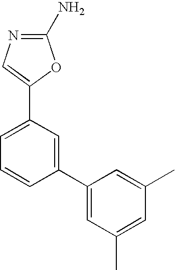

was purified by HPLC using a gradient of 20 to 80% acetonitrile in water at 1% per minute, to afford the title compound (80 mg, 25%) as a white solid, LC/MS System B: Rt=6.61 min, m/z (ES⁺)=265 ((M+H) for C₁₇H₁₆N₂O), ¹H NMR (DMSO-D6) 2.3 (6H, s), 7.0 (1H, s), 7.25 (2H, m), 7.45-7.55 (3H, m), 7.65 (1H, s), 7.7 (1H, m).
5-(4′-Ethyl-biphenyl-3-yl)-oxazol-2-ylamine, trifluoroacetic acid (209L)

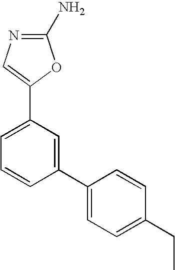

was purified by HPLC using a gradient of 20 to 80% acetonitrile in water at 1% per minute, to afford the title compound (15 mg, 5%) as a white solid, LC/MS System B: Rt=6.66 min, m/z (ES⁺)=265 ((M+H) for C₁₇H₁₆N₂O) ¹H NMR (DMSO-D6) 1.2 (3H, t, J=7.5 Hz), 2.6 (2H, q, J=7.5 Hz), 7.3 (2H, m), 7.35-7.45 (4H, m), 7.55 (2H, m), 7.7 (1H, m).
5-(3′-Isopropyl-biphenyl-3-yl)-oxazol-2-ylamine, trifluoroacetic acid (209M)

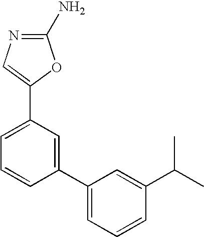

was purified by HPLC using a gradient of 20 to 80% acetonitrile in water at 1% per minute, to afford the title compound (8 mg 2%) as a white solid, LC/MS System B: Rt=6.84 min, m/z (ES⁺)=279 ((M+H) for C₁₈H₁₈N₂O), ¹H NMR (DMSO-D6) 1.2 (6H, d, J=6.8 Hz), 2.9 (1H, m), 7.3 (2H, m), 7.4-7.6 (5H, m), 7.65 (1H, m), 7.75 (1H, m).

### Example 3(c)

### Synthesis of 5-biphenyl-3-yl-oxazol-2-ylamine, trifluoroacetic acid (211)

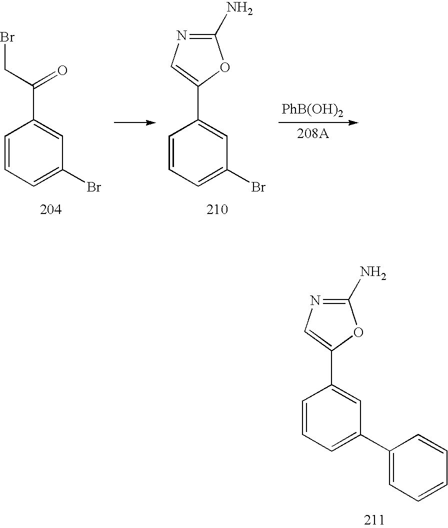 [0490] (3-Bromo-phenyl)-oxazol-2-ylamine (210)

A solution of 2-bromo-1-(3-bromo-phenyl)-ethanone (Compound 204, 6.8 g), urea (4.4 g) and N,N-dimethylformamide (70 mL) was split equally between 14 microwave vials. The vessels were equipped with a stirrer bar, sealed with a crimped septum cap, and placed in the microwave cavity. The vials were heated at 180° C. for 3 minutes, after this time the vials were allowed to cool to room temperature, and the resultant mixtures were combined in a round bottom flask, and the solvent removed under reduced pressure. The residue was partitioned between ethyl acetate (100 mL) and water (50 mL). The organic layer was washed with brine (2×50 mL), dried over magnesium sulfate and the solvent removed under reduced pressure to give a yellow solid. The crude product was purified by column chromatography, eluting with 5 to 30% ethyl acetate in cyclohexane, to afford 5-(3-bromo-phenyl)-oxazol-2-ylamine compound (1.1 g, 9%) as a yellow solid. LC/MS System B: Rt=5.97 min, m/z (ES⁺)=239, 241 ((M+H) for C₉H₇BrN₂O). ¹H NMR (DMSO-D6) 6.7 (2H, br s), 7.25 (1H, t, J=7.9 Hz), 7.35 (1H, ddd, J=7.9, 1.9, 1.1 Hz), 7.55 (1H, ddd, J=7.9, 1.3, 1.1, Hz), 7.75 (1H, t, J=1.9 Hz), 7.9 (1H, s).
5-Biphenyl-3-yl-oxazol-2-ylamine, trifluoroacetic acid (211)

In a microwave vial was placed 5-(3-bromo-phenyl)-oxazol-2-ylamine (Compound 210, 500 mg), benzeneboronic acid (Compound 208A, 378 mg), palladium (0) tetrakis (triphenylphosphine) (97 mg), 2M cesium carbonate (4.2 mL), and N,N-dimethylformamide (3 mL). The vial was heated at 100° C. for 3 minutes, allowed to cool to room temperature and then concentrated under reduced pressure. The residue was partitioned between water (5 mL) and dichloromethane (5 mL). The organic layer was washed with water (5 mL), dried over magnesium sulfate and concentrated under reduced pressure. The crude product was purified by HPLC using a gradient of 20 to 80% acetonitrile in water at 1% per minute, to afford 5-biphenyl-3-yl-oxazol-2-ylamine, trifluoroacetic acid (39 mg, 5%) as a white solid. LC/MS System B: Rt=6.33 min, m/z (ES⁺)=237 ((M+H) for C₁₅H₁₂N₂O). ¹H NMR (DMSO-D6) 6.7 (2H, br s), 7.25-7.65 (8H, m), 7.90 (1H, m), 7.95 (1H, s).

### Example 3(d)

### Synthesis of intermediates 4-(3-bromo-phenyl) 5-methyl-oxazol-2-ylamine (216) and 5-(3-bromo-phenyl)-4-methyl-oxazol-2-ylamine (217)

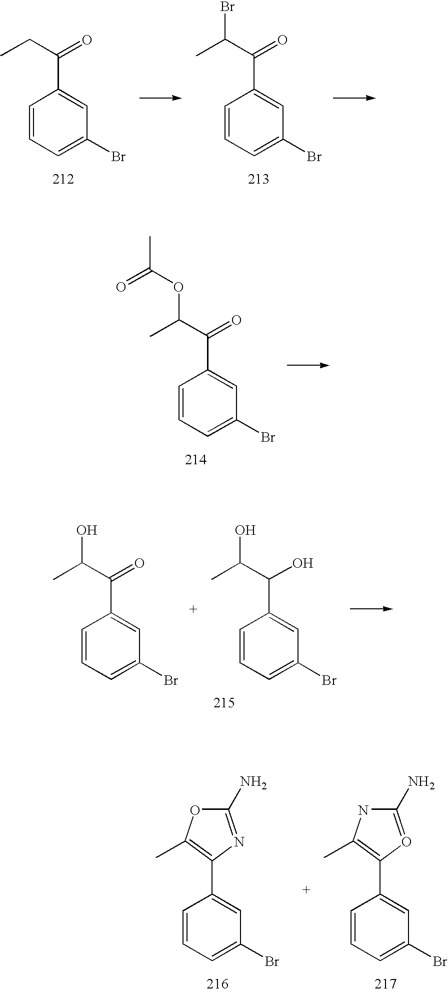 [0495] 2-Bromo-1-(3-bromo-phenyl)-propan-1-one (213)

To a solution of in 1-(3-bromo-phenyl)-propan-1-one (Compound 212, 25.1 g) 1,2-dimethoxyethane (250 mL) at 0° C. was added phenyl trimethylammonium tribromide (47.7 g). The mixture was stirred at 0° C. for 10 minutes and then at room temperature for 2 hours. The mixture was diluted with ethyl acetate (300 mL), washed with water (200 mL), dried over magnesium sulfate and the solvent removed under reduced pressure to afford 2-bromo-1-(3-bromo-phenyl)-propan-1-one (33.6 g, 97%) as an orange oil. ¹H NMR (CDCl₃) 1.85 (3H, d, J=6.6 Hz), 5.2 (1H, q, J=6.6 Hz), 7.35 (1H, t, J=8.0 Hz), 7.7 (1H, ddd, J=8.0, 2.0, 1.0 Hz), 7.9 (1H, ddd, J=8.0, 1.8, 1.0 Hz), 8.1 (1H, t, J=1.8 Hz).
Acetic acid 2-(3-bromo-phenyl)-1-methyl-2-oxo-ethyl ester (214)

A mixture of 2-bromo-1-(3-bromo-phenyl)-propan-1-one (Compound 213, 30.1 g), sodium acetate (8.4 g) and N,N-dimethylformamide (350 mL) was heated at 90° C. for 2 hours. The N,N-dimethylformamide was removed under reduced pressure and the residue was partitioned between water (300 mL) and dichloromethane (300 mL). The organic layer was washed with water, dried over magnesium sulfate and concentrated under reduced pressure to afford acetic acid 2-(3-bromo-phenyl)-1-methyl-2-oxo-ethyl ester (25.1 g, 90%) as a dark orange liquid. ¹H NMR (CDCl₃) 1.5 (3H, d, J=7.0 Hz), 2.1 (3H, s), 5.8 (1H, q, J=7.0 Hz), 7.3 (1H, t, J=7.9 Hz), 7.7 (1H, ddd, J=7.9, 1.8, 1.1 Hz), 7.8 (1H, m), 8.0 (1H, t, J=1.8 Hz).
1-(3-bromo-phenyl)-2-hydroxy-propan-1-one and 1-(3-bromo-phenyl)-1-hydroxy-propan-2-one (215)

A mixture of acetic acid 2-(3-bromo-phenyl)-1-methyl-2-oxo-ethyl ester (Compound 214, 25.1 g), industrial methylated spirits (150 mL) and 1 M hydrochloric acid (120 mL) was heated at reflux for 2 hours. The solvent was removed under reduced pressure and the crude material was partitioned between water (100 mL) and ethyl acetate (100 mL). The organic layer was washed with water, dried over magnesium sulfate and concentrated under reduced pressure to afford a 2:1 mixture of 1-(3-bromo-phenyl)-2-hydroxy-propan-1-one and 1-(3-bromo-phenyl)-1-hydroxy-propan-2-one (18.8 g, 89%) as a dark orange oil. ¹H NMR (CDCl₃) 1.4 (3H, d, J=7.0 Hz), 2.1 (3H, s), 3.6 (1H, d, J=6.4 Hz), 4.3 (1H, d, J=4.2 Hz), 5.0 (1H, d, J=4.2 Hz), 5.05 (1H, m), 7.20-7.45 (5H, m), 7.7 (1H, ddd, J=8.0, 2.0, 1.0 Hz), 7.8 (1H, m), 8.0 (1H, J=1.8 Hz).
4-(3-bromo-phenyl)-5-methyl-oxazol-2-ylamine (216) and 5-(3-bromo-phenyl)-4-methyl-oxazol-2-ylamine (217)

A solution of 1-(3-bromo-phenyl)-2-hydroxy-propan-1-one and 1-(3-bromo-phenyl)-1-hydroxy-propan-2-one (2:1 mixture, Compound 215, 18.8 g), cyanamide (10.4 g) and N,N-dimethylformamide (180 mL) was split equally between 40 microwave vials. The vessels were equipped with a stirrer bar, sealed with a crimped septum cap, and placed in the microwave cavity. The vials were heated at 200° C. for 10 minutes, after this time the vials were allowed to cool to room temperature, and the resultant mixtures were combined in a round bottom flask, and concentrated under reduced pressure. The residue was purified by column chromatography, eluting with 50 to 90% ethyl acetate in dichloromethane to afford 4-(3-bromo-phenyl)-5-methyl-oxazol-2-ylamine (1.4 g, 7%) as an orange solid. LC/MS System B: Rₜ=4.74 min, m/z (ES⁺)=253, 255 ((M+H) for C₁₀H₉BrN₂O), ¹H NMR (DMSO-D6) 2.35 (3H, s), 6.5 (2H, br s), 7.3 (1H, t, J=7.8 Hz), 7.4 (1H, ddd, J=8.0, 2.1, 1.1 Hz), 7.5 (1H, m), 7.7 (1H, t, J=1.8 Hz), then with 5% methanol in dichloromethane to afford 5-(3-bromo-phenyl)-4-methyl-oxazol-2-ylamine (4.1 g, 20%) as a cream solid. LC/MS System B: Rt=4.55 min, m/z (ES⁺)=253, 255 ((M+H) for C₁₀H₉BrN₂O). ¹H NMR (DMSO-D6) 2.15 (3H, s), 6.8 (2H, br s), 7.3-7.4 (3H, m), 7.45 (1H, m).

### Example 3(e)

### Synthesis of 4-biphenyl-3-yl-5-methyl-oxazol-2-ylamines

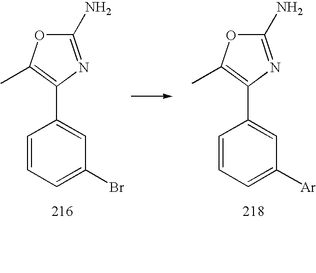 [0504] 4-Biphenyl-3-yl-5-methyl-oxazol-2-ylamine, trifluoroacetic acid (218A)

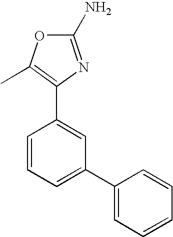

In a microwave vial was placed 4-(3-bromo-phenyl)-5-methyl-oxazol-2-ylamine (Compound 216, 0.2 g), benzeneboronic acid (Compound 208A, 140 mg), palladium (0) tetrakis (triphenylphosphine) (36 mg), 2M cesium carbonate (1.6 mL), and N,N-dimethylformamide (2.4 mL). The vial was heated at 100° C. for 3 minutes, allowed to cool to room temperature and then filtered through a short pad of hyflo. The filtrate was concentrated under reduced pressure and the residue was purified by HPLC using a gradient of 20 to 80% acetonitrile in water at 1% per minute, to afford 4-biphenyl-3-yl-5-methyl-oxazol-2-ylamine, trifluoroacetic acid (22 mg, 8%) as a pink solid. LC/MS System B: Rt=5.41 min, m/z (ES⁺)=251 ((M+H) for C₁₆H₁₄N₂O). ¹H NMR (CDCl₃) 2.4 (3H, s), 7.35 (1H, m), 7.45-7.55 (4H, m), 7.6-7.7 (3H, m), 7.75 (1H, m).

### Compounds 218B and 218C

Similarly, replacing benzeneboronic acid (Compound 208A) with other compounds of formula (208):
3-methylbenzeneboronic acid (Compound 208B); and
2-methoxybenzeneboronic acid (Compound 208G);
   and following the procedures of preparation of Compound 218A above, the following compounds of the formula (218) were prepared:
5-Methyl-4-(3′-methyl-biphenyl-3-yl)-oxazol-2-ylamine, trifluoroacetic acid (218B)

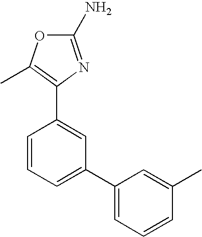

was purified by HPLC using a gradient of 20 to 80% acetonitrile in water at 1% per minute, to afford the title compound (10 mg, 3%) as a pink solid, LC/MS System B: Rt=5.85 min, m/z (ES⁺) 265 ((M+H) for C₁₇H₁₆N₂O), ¹H NMR (DMSO-D6) 2.35 (3H, s), 2.40 (3H, s), 7.15 (1H, d, J=7.5 Hz), 7.35 (1H, t, J=7.5 Hz), 7.45-7.55 (4H, m), 7.6 (1H, m), 7.75 (1H, m).
4-(2′-methoxy-biphenyl-3-yl)-5-methyl-oxazol-2-ylamine, trifluoroacetic acid (218C)

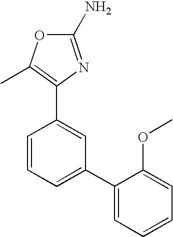

was purified by HPLC using a gradient of 20 to 80% acetonitrile in water at 1% per minute, to afford the title compound (145 mg, 46%) as a purple solid. LC/MS System B: Rt=5.33 min, m/z (ES⁺)=281 ((M+H) for C₁₇H₁₆N₂O₂). ¹H NMR (DMSO-D6) 2.35 (3H, s), 3.75 (3H, s), 7.0 (1H, m), 7.1 (1H, d, J=7.1 Hz), 7.3-7.5 (5H, m), 7.6 (1H, m).

### Example 3(f)

### Synthesis of 5-biphenyl-3-yl-4-methyl-oxazol-2-ylamines

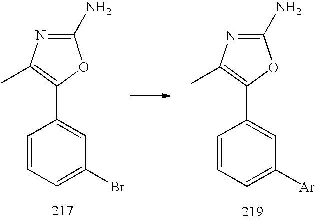 [0512] 5-biphenyl-3-yl-4-methyl-oxazol-2-ylamine, trifluoroacetic acid (219A)

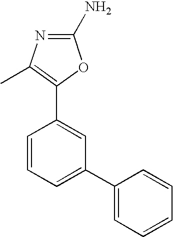

In a microwave vial was placed 5-(3-bromo-phenyl)-4-methyl-oxazol-2-ylamine (Compound 217, 0.2 g), benzeneboronic acid (Compound 208A, 140 mg), palladium (0) tetrakis (triphenylphosphine) (36 mg), 2M cesium carbonate (1.6 mL), and N,N-dimethylformamide (2.4 mL). The vial was heated to 100° C. for 3 minutes, allowed to cool to room temperature and then filtered through a short pad of hyflo. The filtrate was concentrated under reduced pressure and the residue was purified by HPLC using a gradient of 20 to 80% acetonitrile in water at 1% per minute, to afford 5-biphenyl-3-yl-4-methyl-oxazol-2-ylamine, trifluoroacetic acid (208 mg, 71%) as a white solid. LC/MS System B: Rt=5.34 min, m/z (ES⁺)=251 ((M+H) for C₁₆H₁₄N₂O). ¹H NMR (DMSO-D6) 2.3 (3H, s), 7.35 (1H, m), 7.4-7.5 (3H, m), 7.5-7.6 (2H, m), 7.60-7.65 (3H, m).
5-(2′-Methoxy-biphenyl-3-yl)-4-methyl-oxazol-2-ylamine, trifluoroacetic acid (219B)

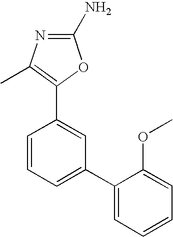

Similarly, replacing benzeneboronic acid with 2-methoxybenzeneboronic acid (Compound 208G) and following the procedures of preparation of Compound 219A above, 5-(2′-methoxy-biphenyl-3-yl)-4-methyl-oxazol-2-ylamine, trifluoroacetic acid (153 mg, 49%) was prepared as a white solid. LC/MS System B: Rt=5.24 min, m/z (ES⁺)=281 ((M+H) for C₁₇H₁₆N₂O₂). ¹H NMR (DMSO-D6) 2.25 (3H, s), 3.75 (3H, s), 7.0 (1H, td, J=7.5, 0.9 Hz), 7.1 (1H, d, J=7.6 Hz), 7.3 (1H, dd, J=7.6, 1.8 Hz), 7.3-7.4 (3H, m), 7.45-7.55 (2H, m).

### Example 3(g)

### Synthesis of 5-(4-methoxy-biphenyl-3-yl)-oxazol-2-ylamines

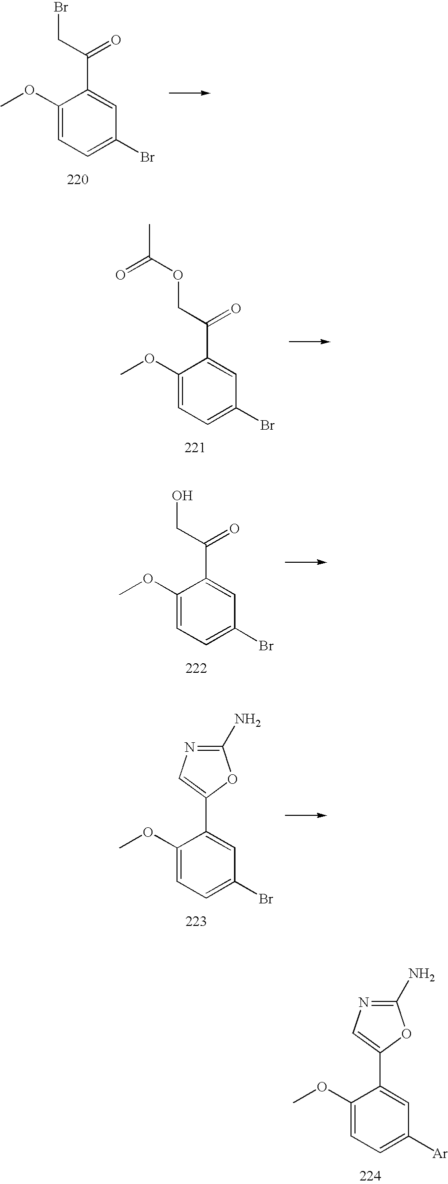 [0517] Acetic acid 2-(5-bromo-2-methoxy-phenyl)-2-oxo-ethyl ester (221)

A mixture of 2-bromo-1-(5-bromo-2-methoxy-phenyl)-ethanone (Compound 220, 10.0 g), sodium acetate (2.7 g) and N,N-dimethylformamide (110 mL) was heated at 80° C. for 2 hours. The N,N-dimethylformamide was removed under reduced pressure and the residue was partitioned between water (100 mL) and dichloromethane (100 mL). The organic layer was washed with water (100 mL), brine (100 mL) and dried over magnesium sulfate. The solvent was removed under reduced pressure to afford acetic acid 2-(5-bromo-2-methoxy-phenyl)-2-oxo-ethyl ester (9.2 g, 99%) as a dark orange oil. ¹H NMR (CDCl₃) 2.15 (3H, s), 3.9 (3H, s), 5.15 (2H, s), 6.85 (1H, d, J=8.8 Hz), 7.55 (1H, dd, J=8.8, 2.6 Hz), 8.0 (1H, d, J=2.6 Hz).
1-(5-Bromo-2-methoxy-phenyl)-2-hydroxy-ethanone (222)

A mixture of acetic acid 2-(5-bromo-2-methoxy-phenyl)-2-oxo-ethyl ester (Compound 221, 9.2 g) industrial methylated spirits (50 mL) and 1 M hydrochloric acid (40 mL) was heated at reflux for 2 hours. The solvent was removed under reduced pressure and the residue was partitioned between water (50 mL) and ethyl acetate (50 mL). The organic layer was washed with water (50 mL) and dried over magnesium sulfate. The solvent was removed under reduced pressure to afford 1-(5-bromo-2-methoxy-phenyl)-2-hydroxy-ethanone (6.5 g, 83%) as a yellow solid. ¹H NMR (CDCl₃) 3.6 (1H, t, J=4.8 Hz), 3.9 (3H, s), 4.7 (2H, d, J=4.8 Hz), 6.85 (1H, d, J=8.9 Hz), 7.6 (1H, dd, J=8.9, 2.6 Hz), 8.1 (1H, d, J=2.6 Hz).
5-(5-Bromo-2-methoxy-phenyl) oxazol-2-ylamine (223)

A solution of 1-(5-bromo-2-methoxy-phenyl)-2-hydroxy-ethanone (Compound 222, 6.5 g), cyanamide (3.3 g) and N,N-dimethylformamide (65 mL) was split equally between 13 microwave vials. The vessels were equipped with a stirrer bar, sealed with a crimped septum cap, and placed in the microwave cavity. The vials were heated at 200° C. for 10 minutes, after this time the vials were allowed to cool to room temperature, and the resultant mixtures were combined in a round bottom flask, and concentrated under reduced pressure. The residue was partitioned between ethyl acetate (100 mL) and water (100 mL). The organic layer was washed with water (100 mL), dried over magnesium sulfate and the solvent removed under reduced pressure to afford 4-(5-bromo-2-methoxy-phenyl)oxazol-2-ylamine (6.6 g, 65%) as a dark orange solid. LC/MS System A: Rₜ=2.26 min, m/z (ES⁺)=269, 271 ((M+H) for C₁₀H₉BrN₂O₂). ¹H NMR (DMSO-D6) 3.85 (3H, s), 6.9 (2H, br s), 7.0 (1H, d, J=8.8 Hz), 7.1 (1H, s), 7.3 (1H, dd, J=8.8, 2.4 Hz), 7.4 (1H, d, J=2.4 Hz).
5-(4-methoxy-biphenyl-3-yl)-oxazol-2-ylamine, trifluoroacetic acid (224A)

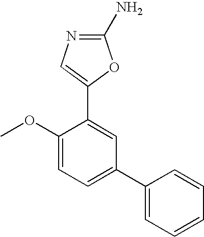

In a microwave vial was placed 5-(5-bromo-2-methoxy-phenyl)oxazol-2-ylamine (Compound 223, 200 mg), benzeneboronic acid (Compound 208A, 136 mg), palladium (0) tetrakis(triphenylphosphine) (34 mg), 2M cesium carbonate (1.5 mL), and dimethylformamide (3 mL). The vial was heated to 120° C. for 3 minutes, allowed to cool to room temperature and then filtered through a short pad of hyflo. The filtrate was concentrated under reduced pressure and the residue was purified by HPLC using a gradient of 20 to 80% acetonitrile in water at 1% per minute, to afford 5-(4-methoxy-biphenyl-3-yl)-oxazol-2-ylamine, trifluoroacetic acid (32 mg, 11%) as a white solid. LC/MS System B: Rt=5.41 min, m/z (ES⁺)=267 ((M+H) for C₁₆H₁₄N₂O₂)

### Compounds 224B and 224C

Similarly, replacing benzeneboronic acid (Compound 208B) with other compounds of formula (208):
2-methoxybenzeneboronic acid (Compound 208G); and
3-methylbenzeneboronic acid (Compound 208B)
   and following the procedures of preparation of Compound 224A above, the following compounds of the formula (224) were prepared:
5-(4,2′-dimethoxy-biphenyl-3-yl)-oxazol-2-ylamine, trifluoroacetic acid (224B)

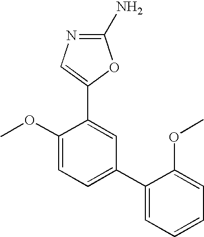

was purified by HPLC using a gradient of 20 to 80% acetonitrile in water at 1′ per minute, to afford the title compound (29 mg, 10%) as a white solid, LC/MS System B: Rt=5.39 min, m/z (ES⁺)=297 ((M+H) for C₁₇H₁₆N₂O₃), ¹H NMR (DMSO-D6) 3.7 (3H, s), 3.9 (3H, s), 7.0 (1H, td, J=7.4, 0.8 Hz), 7.05 (1H, d, J=7.9 Hz), 7.1 (1H, d, J=8.6 Hz), 7.2-7.3 (2H, m), 7.35-7.40 (2H, m), 7.55 (1H, m)
5-(4-Methoxy-3′-methyl-biphenyl-3-yl)-oxazol-2-ylamine, trifluoroacetic acid (224C)

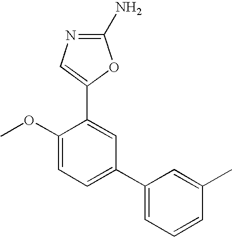

was purified by HPLC using a gradient of 20 to 80% acetonitrile in water at 1% per minute, to afford the title compound (18 mg, 9%) as an off-white solid, LC/MS System B: Rt=2.71 min, m/z (ES⁺)=281 ((M+H) for C₁₇H₁₆N₂O₂), ¹H NMR (DMSO-D6) 2.35 (3H, s), 3.9 (3H, s), 7.1 (1H, d, J=7.2 Hz), 7.2 (1H, d, J=8.6 Hz), 7.3-7.4 (4H, m), 7.55 (1H, dd, J=8.6, 2.4 Hz), 7.65 (1H, d, J=2.2 Hz).

### EXAMPLE 4

### Example 4(a)

### Synthesis of 1-methyl-N⁵-biphenyl-1H-[1, 2,4]triazole-3,5-diamines

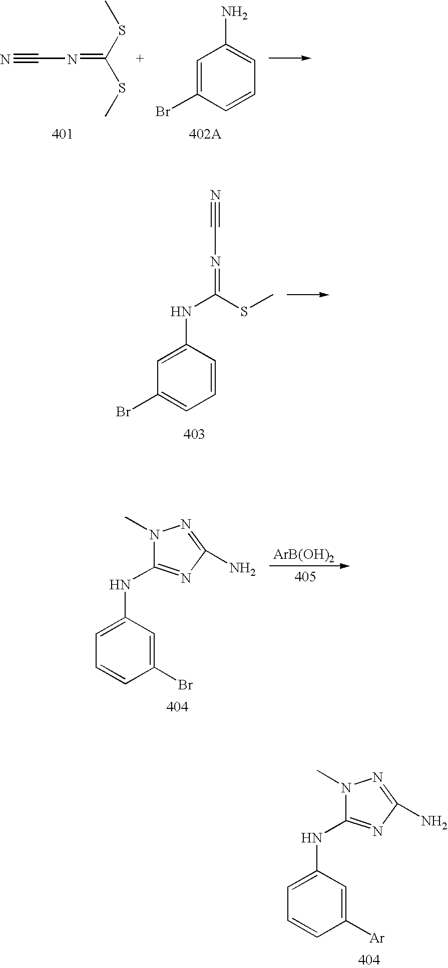 [0531] Methyl N′-cyano-N-(3-bromophenyl) carbamimidothioate (403)

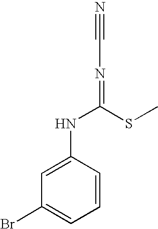

A mixture of dimethyl N-cyanodithioiminocarbonate (401, 10.0 g), 3-bromoaniline (402A, 5.6 g) and pyridine (50 mL) was heated at reflux for 3 hours. The solvent was removed under reduced pressure and the residue was washed with ethanol (200 mL) and diethyl ether (100 mL) to afford the title compound (5.4 g, 61%) as a white solid. ¹H NMR (DMSO-D6) 2.65 (3H, s), 7.3 (1H, t, J=8.0 Hz), 7.40 (1H, m), 7.45 (1H, m), 7.65 (1H, t, J=1.9 Hz).
N⁵-(3-bromo-phenyl)-1-methyl-1H-[1,2,4]triazole-3,5-diamine (404)

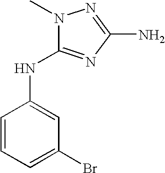

A mixture of methyl N′-cyano-N-(3-bromophenyl) carbamimidothioate (403, 2.7 g), methylhydrazine (0.92 g) and butanol (10 mL) was heated at reflux for 2 hours. The solvent was removed under reduced pressure to give a yellow solid. The solid was recrystallised from ethyl acetate and washed with hexane to afford the title compound (1.5 g, 56%) as a pale yellow solid. LC/MS System B: Rt=2.33 min, m/z (ES⁺)=267/269 ((M+H) for C₉H₁₀BrN₅). ¹H NMR (DMSO-D6) 3.3 (3H, s), 5.05 (2H, br s), 6.95 (1H, m), 7.15 (1H, t, J=8.1 Hz), 7.4 (1H, m), 7.85 (1H, t, J=2.0 Hz), 8.8 (1H, br s).
N⁵-biphenyl-3-yl-1-methyl-1H-[1,2,4]triazole-3,5-diamine (406A)

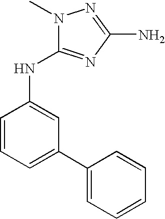

In a microwave vial was placed N⁵-(3-bromo-phenyl)-1-methyl-1H-[1,2,4]triazole-3,5-diamine (404, 250 mg), benzeneboronic acid (Compound 405A, 170 mg), palladium (0) tetrakis(triphenylphosphine) (46 mg), 2M cesium carbonate (1.5 mL), and N,N-dimethylformamide (2 mL). The vial was heated to 100° C. for 3 minutes, allowed to cool to room temperature and the solvent removed under reduced pressure. The residue was dissolved in ethyl acetate and filtered through a short pad of hyflo to remove palladium residues. The filtrate was concentrated under reduced pressure and purified by column chromatography, eluting with 10% ethanol in ethyl acetate, to give a white solid. The solid was recrystallised from a mixture of ethyl acetate and hexane to afford the title compound (46 mg, 19%) as a white solid. LC/MS System B: Rt=2.62 min, m/z (ES⁺)=266 ((M+H) for C₁₅H₁₅N₅). ¹H NMR (DMSO-D6) 3.3 (3H, s), 5.0 (2H, br s), 7.1 (1H, m), 7.25-7.35 (2H, m), 7.4 (2H, m), 7.5-7.6 (3H, m), 7.75 (1H, t, J=1.9 Hz), 8.65 (1H, br s).

### Compounds 406B-406C

Similarly, replacing benzeneboronic acid with other compounds of formula (405):
2-methoxybenzeneboronic acid (Compound 405B);
3-methylbenzeneboronic acid (Compound 405C);
   and following the procedures of preparation of 406A above, the following compounds of the formula (406) were prepared:
N⁵-(2′-methoxy-biphenyl-3-yl)-1-methyl-1H-[1,2,4]triazole-3,5-diamine (406B)

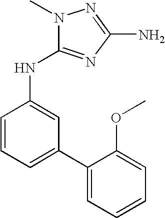

as a white solid (108 mg, 39%); LC/MS System B: Rt=2.57 min, m/z (ES⁺)=296 ((M+H) for C₁₆H₁₇N₅O), ¹H NMR (DMSO-D6) 3.25 (3H, s), 3.7 (3H, s), 4.95 (2H, br s), 6.9 (1H, m), 7.0 (1H, td, J=7.4, 1.0 Hz), 7.05 (1H, m), 7.15-7.25 (2H, m), 7.3 (1H, m), 7.45 (1H, m), 7.55 (1H, t, J=1.9 Hz), 8.6 (1H, br s).
1-methyl-1-(3′-methyl-biphenyl-3-yl)-1H-[1,2,4]triazole-3,5-diamine (406C)

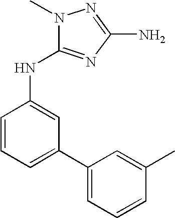

as a white solid (123 mg, 47%), LC/MS System B: Rt=2.81 min, m/z (ES⁺)=280 ((M+H) for C₁₆H₁₇N₅), ¹H NMR (DMSO-D6) 2.35 (3H, s), 3.25 (3H, s), 5.0 (2H, br s), 7.05-7.15 (2H, m), 7.25-7.40 (4H, m), 7.55 (1H, m), 7.7 (1H, t, J=2.0 Hz), 8.6 (1H, br s).

### Example 4(b)

### Synthesis of 1-methyl-N³-biphenyl-1H-[1,2,4]triazole-3,5-diamines and 1-methyl-N⁵-biphenyl-1H-[1,2,4]triazole-3,5-diamines

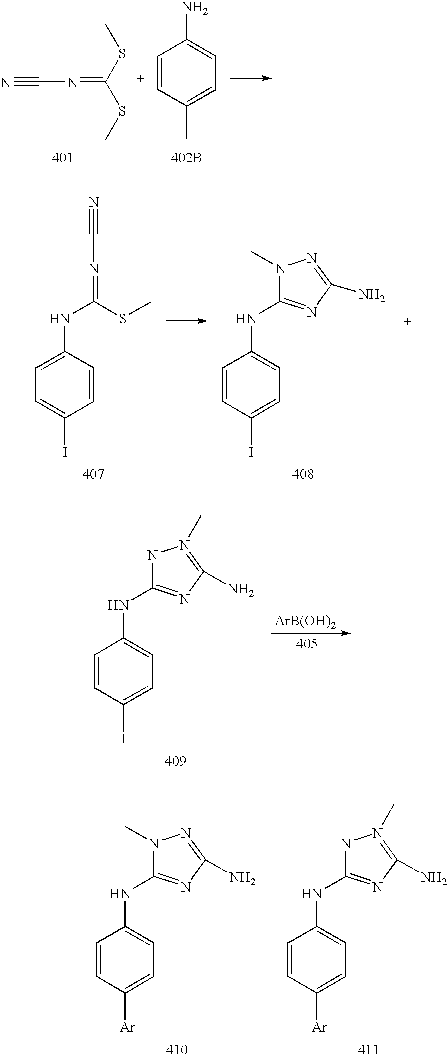 [0543] Methyl N′-cyano-N-(4-iodophenyl) carbamimidothioate (407)

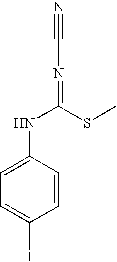

A mixture of dimethyl N-cyanodithioiminocarbonate (401, 10.0 g), 4-iodoaniline (402B, 7.1 g) and pyridine (50 mL) was heated at reflux for 3 hours. The solvent was removed under reduced pressure and the residue was washed with industrial methylated spirits (300 mL) and diethyl ether (100 mL) to afford the title compound (6.9 g, 67w) as a white solid. ¹H NMR (DMSO-D6) 2.65 (3H, s), 7.25 (2H, m), 7.7 (2H, m), 10.1 (1H, br s)
N⁵-(4-iodo-phenyl)-1-methyl-1H-[1,2,4]triazole-3,5-diamine (408) and N³-(4-iodo-phenyl)-1-methyl-1H-[1,2,4]triazole-3,5-diamine (409)

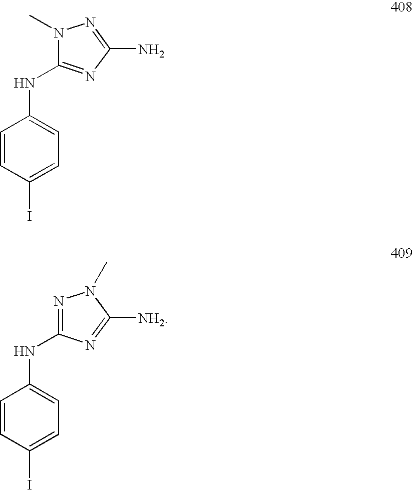

A mixture of methyl N′-cyano-N-(4-iodophenyl) carbamimidothioate (407, 3.0 g), methylhydrazine (0.87 g) and butanol (40 mL) was heated at reflux for 2 hours. The solvent was removed under reduced pressure to give a 2:1 mixture of N-(4-iodo-phenyl)-1-methyl-1H-[1,2,4]triazole-3,5-diamine and N³— (4-iodo-phenyl)-1-methyl-1H-[1,2,4]triazole-3,5-diamine (3.1 g, 100%) as a peach solid. A portion of the solid (100 mg) was purified by HPLC using a gradient of 20 to 80% acetonitrile in water at 1% per minute, to afford N⁵-(4-iodo-phenyl)-1-methyl-1H-[1,2,4]triazole-3,5-diamine trifluoroacetic acid (59 mg, 59%) as a white solid, LC/MS System B: Rt=2.44 min, m/z (ES⁺)=316 ((M+H) for C₉H₁₀IN₅), ¹H NMR (DMSO-D6) 3.45 (3H, s), 7.3 (2H, m), 7.55 (2H, m), 9.0 (1H, br s) and N³-(4-iodo-phenyl)-1-methyl-1H-[1,2,4]triazole-3,5-diamine trifluoroacetic acid (27 mg, 275) as a white solid, LC/MS System B: Rt=2.49 min, m/z (ES⁺)=316 ((M+H) for C₉H₁₀IN₅) ¹H NMR (DMSO-D6) 3.4 (3H, s), 7.25 (2H, m), 7.5 (2H, m), 9.2 (1H, br s).
N⁵-biphenyl-4-yl-1-methyl-1H-[1,2,4]triazole-3,5-diamine trifluoroacetic acid (410A) and N³-biphenyl-4-yl-1-methyl-1H-[1,2,4]triazole-3,5-diamine trifluoroacetic acid (411A)

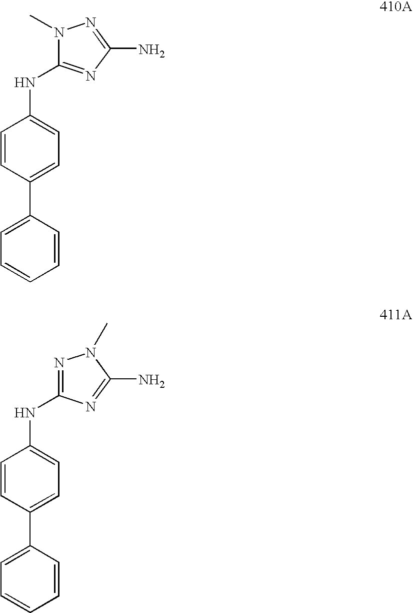

In a microwave vial was placed a 2:1 mixture of N⁵-(4-iodo-phenyl)-1-methyl-1H-[1,2,4]triazole-3,5-diamine and N³-(4-iodo-phenyl)-1-methyl-1H-[1,2,4]triazole-3,5-diamine (408 and 409, 200 mg), benzeneboronic acid (405A, 120 mg), palladium (0) tetrakis(triphenylphosphine) (29 mg), 2M cesium carbonate (1.3 mL), and N,N-dimethylformamide (2.5 mL). The vial was heated to 100° C. for 3 minutes, allowed to cool to room temperature and filtered through a short pad of hyflo to remove palladium residues. The solvent was removed under reduced pressure and the residue was purified by HPLC using a gradient of 20 to 80% acetonitrile in water at 1% per minute, to afford N⁵-biphenyl-4-yl-1-methyl-1H-[1,2,4]triazole-3,5-diamine trifluoroacetic acid (13 mg, 8%) as an off-white solid, LC/MS System B: Rt=2.64 min, m/z (ES⁺)=266 ((M+H) for C₁₅H₁₅N₅), ¹H NMR (DMSO-D6) 3.5 (3H, s), 7.25 (1H, m), 7.4 (2H, m), 7.5-7.6 (6H, m), 9.2 (1H, br s) and N³-biphenyl-4-yl-1-methyl-1H-[1,2,4]triazole-3,5-diamine trifluoroacetic acid (8 mg, 5%) as a white solid, LC/MS System B: Rₜ=2.63 min, m/z (ES⁺)=266 ((M+H) for C₁₅H₁₅N₅) ¹H NMR (DMSO-D6) 3.45 (3H, s), 7.25 (1H, m), 7.35 (2H, m), 7.5-7.6 (6H, m), 9.2 (1H, br s).

### Compounds 410B-410C and 411B-411C

Similarly, replacing benzeneboronic acid with other compounds of formula (405):
2-methoxybenzeneboronic acid (Compound 405B);
3-methylbenzeneboronic acid (Compound 405C);
   and following the procedures of preparation of 410A and 411A above, the following compounds of the formula (410) and (411) were prepared:
N⁵-(2′-methoxy-biphenyl-4-yl)-1-methyl-1H-[1,2,4]triazole-3,5-diamine trifluoroacetic acid (410B)

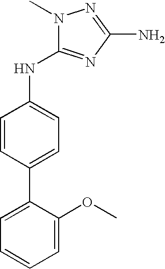

as a yellow solid (125 mg, 26%), LC/MS System B: Rt=2.61 min, m/z (ES⁺)=296 ((M+H) for C₁₆H₁₇N₅O), ¹H NMR (DMSO-D6) 3.5 (3H, s), 3.7 (3H, s), 6.95 (1H, td, J=7.4, 1.1 Hz), 7.05 (1H, m), 7.2-7.3 (2H, m), 7.35-7.45 (4H, m), 9.2 (1H, br s).
N³-(2′-methoxy-biphenyl-4-yl)-7-methyl-1H-[1,2,4]triazole-3,5-diamine trifluoroacetic acid (411B)

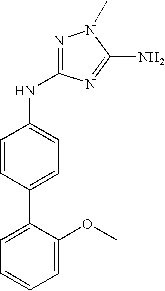

as a pale yellow solid (68 mg, 14%), LC/MS System B: Rt=2.59 min, m/z (ES⁺)=296 ((M+H) for C₁₆H₁₇N₅O), ¹H NMR (DMSO-D6) 3.45 (3H, s), 3.7 (3H, s), 6.95 (1H, td, J=7.4, 1.0 Hz), 7.0 (1H, m), 7.20-7.25 (2H, m), 7.30-7.35 (2H, m), 7.40-7.45 (2H, m), 9.25 (1H, br s).
1-methyl-N⁵-(3′-methyl-biphenyl-4-yl)-1H-[1,2,4]triazole-3,5-diamine trifluoroacetic acid (410C)

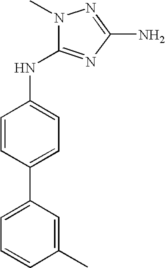

as an off-white solid (17 mg, 4%), LC/MS System B: Rt=2.84 min, m/z (ES⁺)=280 ((M+H) for C₁₆H₁₇N₅), ¹H NMR (DMSO-D6) 2.3 (3H, s), 3.5 (3H, s), 7.1 (1H, m), 7.25 (1H, t, J=7.6 Hz), 7.35-7.40 (2H, m), 7.45-7.55 (4H, m), 9.05 (1H, br s).
1-methyl-N³-(3′-methyl-biphenyl-4-yl)-1H-[1,2,4]triazole-3,5-diamine trifluoroacetic acid (411C)

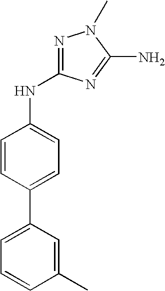

as an off-white solid (20 mg, 4%), LC/MS System B: Rt=2.80 min, m/z (ES⁺)=280 ((M+H) for C₁₆H₁₇N₅), ¹H NMR (DMSO-D6) 2.3 (3H, s), 3.45 (3H, s), 7.05 (1H, m), 7.25 (1H, t, J=7.6 Hz), 7.35 (2H, m), 7.45-7.50 (4H, m), 9.2 (1H, br s).

### Human Cloned 5-HT_{2B} Receptor Binding Assay

The binding affinity of the compounds for human cloned 5-HT_{2B} receptors was determined using the following assay.

CHO-K1 cells expressing cloned 5-HT_{2B} receptor were maintained in Ultra-CHO medium containing 400 μg/ml of G418, 100 U/ml penicillin, 100 μg/ml streptomycin, 2.5 μg/ml fungizone and 1-foetal bovine serum, in 95/5% O₂/CO₂ at 37° C. The cells were harvested using 0.25% trypsin and were centrifuged at 800 rpm for 8 minutes. The cells were homogenised in 50 mM HEPES buffer containing 1 mM disodium EDTA and 1 mM PMSF at pH 7.4, using a Dounce homogeniser (20 strokes). The homogenate was centrifuged at 2280 rpm (1000 g) and 4° C. for 10 minutes, after which the supernatant was removed by decanting. The pellet was re-homogenised as above, and the resulting supernatant removed and combined with that already obtained. The supernatant solution was then centrifuged at 18300 rpm (40000 g) for 10 minutes at 4° C. using a Sorvall centrifuge. The supernatant was removed, and the pellet was re-suspended in 50 mM buffer at pH 7.4 using a Ultra-turrax T25 Polytron, before centrifugation again at 40000 g as above. This wash procedure was repeated, after which the membrane preparation was stored at a concentration of 1 mg/ml at −80° C. until use.

The membranes were thawed rapidly and diluted in assay buffer containing Tris-HCl (50 mM, pH 7.4), ascorbic acid (0.1°) and calcium chloride (4 mM). The membranes were homogenised to resuspend them, prior to adding 10 or 15 μg of membranes to assay wells containing [³H]LSD (1 nM), assay buffer (50 mM Tris, 4 mM calcium chloride and 0.1° ascorbic acid) containing pargyline (10 μM), and the test compounds (1×10⁻¹⁰ to 1×10⁻⁴M). Non specific binding was determined in the presence of 100 μM 5-HT. After 30 minutes incubation at 37° C., the assay mixture was filtered through a combination of GF-C and GF-B filters, pre-soaked in 1% polyethyleneimine, using a Brandel cell harvester, and were washed three times using 50 mM Tris-HCl. Radioactivity retained on the filters was determined by liquid scintillation counting. For each test compound, the concentration that inhibited binding of [³H]LSD by 50% was determined using curve fitting software (Prism). Kd values (concentration of LSD required to occupy 50% of the receptor binding sites at equilibrium) determined from saturation binding studies were then used to calculate inhibition dissociation constants (Ki) using the following equation:

$$Ki = \frac{{IC}_{50}}{1 + ( \frac{Radioligand concentration}{Radioligand Kd} )}$$

The results are shown in table 1 below as pKi values. This approach follows that set out in Kenakin, T. P. Pharmacologic analysis of drug-receptor interaction. Raven Press, New York, 2^{nd} Edition.

Human 5-HT_{2A} and 5-HT_{2C} receptor binding assays

The binding affinity of ligands for human 5-HT_{2A} and 5-HT_{2C} receptors was determined using the following assay. These results were then used to determine the selectivity of the test compounds for 5-HT_{2B} receptors, over 5-HT_{2A} and 5-HT_{2C} receptors.

Membrane preparations from CHO-K1 cells expressing the cloned human 5-HT_{2A} receptor were obtained (Euroscreen). The membranes were thawed rapidly and diluted in assay buffer containing Tris-HCl (50 mM, pH 7.7). The membranes were resuspended by homogenisation, prior to adding 15 μg of membranes to assay wells containing [3H]ketanserin (1 nM), assay buffer (50 mM Tris at pH 7.4) containing pargyline (10 μM), and test compounds (1×10⁻¹⁰ to 1×10⁻⁴M). Non specific binding was determined in the presence of 100 μM mianserin. After 15 minutes incubation at 37° C., the assay mixture was filtered through a combination of GF-C and GF-B filters, pre-soaked in 0.05% Brij, using a Brandel cell harvester, and were washed three times using ice cold Tris-HCl buffer (50 mM). Radioactivity retained on the filters was determined by liquid scintillation counting. For each test compound, the concentration that inhibited binding of [³H]ketanserin by 50% was determined using curve fitting software (Prism). Kd values (concentration of ketanserin required to occupy 50% of the receptor binding sites at equilibrium) determined from saturation binding studies were then used to calculate inhibition dissociation constants (Ki) using the following equation:

$$Ki = \frac{{IC}_{50}}{1 + ( \frac{Radioligand concentration}{Radioligand Kd} )}$$

Membrane preparations from CHO-K1 cells expressing the cloned human 5-HT_{2C} receptor were obtained (Euroscreen). The membranes were thawed rapidly and diluted in assay buffer containing Tris-HCl (50 mM, pH 7.7), ascorbic acid (0.1%) and pargyline (10 μM). The membranes were resuspended by homogenisation, prior to adding 6 μg of membranes to assay wells containing [³H]mesulergine (1 nM), assay buffer (50 mM Tris at pH 7.7 and 0.1% ascorbic acid) containing pargyline (10 μM), and test compounds (1×10⁻¹⁰ to 1×10⁻⁴M). Non specific binding was determined in the presence of 100 μM mianserin. After 30 minutes incubation at 37° C., the assay mixture was filtered through a combination of GF-C and GF-B filters, pre-soaked in 1% bovine serum albumin, using a Brandel cell harvester, and were washed three times using ice cold Tris-HCl buffer (50 mM). Radioactivity retained on the filters was determined by liquid scintillation counting. For each test compound, the concentration that inhibited binding of [³H]mesulergine by 50% was determined using curve fitting software (Prism). Kd values (concentration of mesulergine required to occupy 50% of the receptor binding sites at equilibrium) determined from saturation binding studies were then used to calculate inhibition dissociation constants (Ki) using the following equation:

$$Ki = \frac{{IC}_{50}}{1 + ( \frac{Radioligand concentration}{Radioligand Kd} )}$$

The results are shown in table 1 below as pKi values.

TABLE 1

| Compound | 5-HT_{2B} | 5-HT_{2A} | 5-HT_{2C} |
|---|---|---|---|
| 2A | >6 | <5 | <6 |
| 2E | >6 | <5 | <6.5 |
| 2F | >6 | <5 | <5 |
| 2G | >7 | <5.5 | <6.5 |
| 2H | >6 | <5 | <6 |
| 2I | >6 | <5 | <6 |
| 2J | >7 | <5 | <6 |
| 2K | >6 | <5 | <5 |
| 2M | >6 | <6 | <6 |
| 2N | >6 | <6 | <6 |
| 2O | >6 | <6 | <7 |
| 2Q | >6 | <6 | <6 |
| 2R | >6 | <6 | <6 |
| 2S | >7 | <6 | <6.5 |
| 2T | >6 | <6 | <6 |
| 2U | >6 | <6 | <6 |
| 2V | >6 | <6 | <6 |
| 2X | >6 | <6 | <6 |
| 2Y | >7 | <6 | <6 |
| 2AB | >6 | <6 | <6 |
| 2AC | >6 | <6 | <6 |
| 2AD | >6 | <6.5 | <6 |
| 2AE | >7 | <5 | <6.5 |
| 2AF | >6 | <5.5 | <6 |
| 2AJ | >7 | <6 | <6.5 |
| 2AK | >7 | — | — |
| 2AN | >6 | <5 | <6 |
| 2AP | >6 | <6 | <6 |
| 4B | >7 | <5 | <5 |
| 4C | >6 | <5 | <5 |
| 10A | >7 | <7 | <6.5 |
| 10B | >6 | <5 | <5 |
| 10C | >6 | <6.5 | <6.5 |
| 10D | >6 | <6 | <6 |
| 10E | >7 | <7 | <7 |
| 10F | >7 | <6.5 | <7 |
| 10G | >7 | — | — |
| 10H | >7 | <6.5 | <6.5 |
| 10I | >6 | <5 | <5 |
| 11A | >8 | <6 | <7 |
| 11B | >8 | <6 | <7.5 |
| 12 | >7 | <6 | <6 |
| 16 | >6 | <6 | <6 |
| 103A | >7 | <5.5 | <6 |
| 103B | >6 | <5 | <5 |
| 103C | >7 | <6.5 | <6.5 |
| 103E | >7 | <5 | <6 |
| 103F | >7 | <5.5 | <5.5 |
| 103G | >7 | <6 | <6 |
| 103H | >7 | <6 | <6 |
| 103I | >7 | <6 | <6 |
| 103L | >7 | <5 | <5 |
| 103N | >6 | <5 | <5 |
| 103O | >6 | <5 | <5 |
| 103Q | >6 | <5.5 | <6 |
| 104A | >6 | <6 | <7 |
| 104B | >7 | <5.5 | <5.5 |
| 104C | >7 | <6 | <6.5 |
| 104D | >6 | <6.5 | <6 |
| 104E | >7 | <6 | <7 |
| 104F | >7 | <6 | <6 |
| 104G | >7 | <6.5 | <6.5 |
| 104H | >7 | <6.5 | <7 |
| 104I | >7 | <5.5 | <6 |
| 104J | >7 | <6.5 | <7 |
| 104K | >7 | <7 | <6.5 |
| 104O | >6.5 | <5 | <6.5 |
| 104P | >6.5 | <7 | <6.5 |
| 107 | >6 | — | — |
| 108 | >7 | <6.5 | <7 |
| 209A | >6.5 | <5 | <5 |
| 209B | >6.5 | <5 | <5 |
| 209C | >6 | <5 | <5 |
| 209E | >6.5 | <6 | <5 |
| 209G | >7 | <6 | <6 |
| 209J | >6 | <6 | <6 |
| 209K | >6 | <6 | <6 |
| 209M | >6 | — | — |
| 218A | >6 | <6 | <6 |
| 218B | >6 | — | — |
| 218C | >6.5 | <6 | <6 |
| 219A | >6.5 | <6 | <6 |
| 219B | >7 | <6 | <6 |
| 224A | >6 | <6 | <6 |
| 224B | >6.5 | <6 | <6 |
| 224C | >6 | <6 | <6 |
| 406A | >6.5 | <6 | <6 |
| 406B | >6.5 | <6 | <6 |
| 406C | >7 | <6 | <6 |
| 410C | >6 | <6 | <6 |
| 411A | >6 | <6 | <6 |
| 411B | >6 | <6 | <6 |
| 411C | >6 | <6 | <6 |

Human cloned 5-HT_{2B} cell-based functional assay

The following describes an in vitro functional assay using human cloned 5-HT_{2B} receptors to determine the ability of compounds to block the receptor.

CHO.K1 cells expressing cloned 5-HT_{2B} receptor were maintained In Ultra-CHO medium containing 400 μg/ml of G418, 1000 U/ml penicillin, 100 μg/ml streptomycin, 2.5 μg/ml fungizone, in 95/5% O₂/CO₂ at 37° C. Ultra-CHO medium additionally supplemented with 1% foetal bovine serum was used when seeding the cells and removed after 5 hours. Cells were plated in Costar 96 well white, clear-bottomed plate at a density of 50,000 cells per well and incubated for at least 24 hours in 95/5% O₂/CO₂ at 37° C. before running the assay.

Media was removed from the wells and 200 μl of 4 μM Fluo-4 AM added, this was incubated in a Wallace Victor 2V workstation at 37° C. for 30 minutes. The Fluo-4 AM was then removed from the wells, which were then washed twice with 200 μl of buffer (HBSS without calcium/magnesium/phenol red, 20 mM HEPES, 1 mM Ca²⁺, 1 mM Mg²⁺, 2.5 mM probenecid, pH to 7.4), 180 μl of buffer or test compound was added to the well and incubated for 30 minutes. The Victor 2V injectors were used to inject 20 μl of 5-HT after obtaining 10 0.1-second baseline readings at 535 nm, followed by 150 readings.

All test compounds were aliquoted in 100% DMSO at 10 mM and diluted to 1 mM in 50% DMSO, subsequent dilutions were made using buffer. Buffer was also used to dilute the 5-HT. Data were analysed using Microsoft Excel and GraphPad Prism, with the latter used to produce sigmoidal dose-response curves for each compound. The compound concentration that inhibited the 5-HT response by 50% was taken (IC₅₀-M), and the results are shown in Table 2, as pIC₅₀, being the negative log (to the base 10) of the measured IC₅₀ values.

TABLE 2

| Compound | pIC₅₀ |
|---|---|
| 103C | >7 |
| 103F | >7 |
| 104C | >6 |
| 104J | >7 |
| 219B | >6 |
| 406C | >7 |

1-91. (canceled)

92. A compound of formula I: 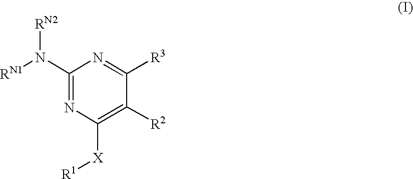
or a salt, solvate and chemically protected form thereof, wherein:
X is O or NH;
R² and R³ are independently selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl;
R¹ is an optionally substituted C₉₋₁₄ aryl group or an optionally substituted bi-C₅₋₇ aryl group;
R^{N1} and R^{N2} are either:
(i) independently selected from H, R, R′, SO₂R, C(═O)R, (CH₂)ₙNR^{N3}R^{N4}, where n is from 1 to 4 and R^{N3} and R^{N4} are independently selected from H and R, where R is optionally substituted C₁₋₄ alkyl, and R′ is optionally substituted phenyl-C₁₋₄ alkyl, or
(ii) together with the nitrogen atom to which they are attached, form an optionally substituted C₅₋₇ heterocyclic group;
with the provisos that when R^{N1}, R^{N2} and R² are H, R³ is methyl, and X is NH, then R¹ is not:

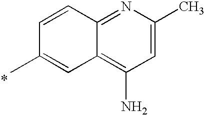

93. The compound according to claim 92, wherein R^{N1} and R^{N2} are both H.

94. The compound according to claim 92, wherein R² is H.

95. The compound according to claim 92, wherein R¹ is an optionally substituted biphenyl group.

96. A compound of formula II: 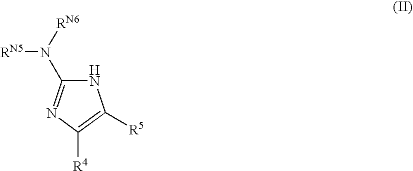
or a salt, solvate and chemically protected form thereof, wherein:
R⁵ is selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl;
R⁴ is an optionally substituted C₉₋₁₄ aryl group or an optionally substituted bi-C₅₋₇ aryl group;
R^{N5} and R^{N6} are either:
(i) independently selected from H, R, R′, SO₂R, C(═O)R, (CH₂)ₙNR^{N7}R^{N8}, where n is from 1 to 4 and R^{N7} and R^{N8} are independently selected from H and R, where R is optionally substituted C₁₋₄ alkyl, and R′ is optionally substituted phenyl-C₁₋₄ alkyl, or
(ii) together with the nitrogen atom to which they are attached, form an optionally substituted C₅₋₇ heterocyclic group;
with the provisos that when R^{N5}, R^{N6} and R⁵ are H, R⁴ is not unsubstituted 1- or 2-naphthyl or unsubstituted 4-phenyl-phenyl
and that when R^{N6} and R⁵ are H, and R^{N5} is acetyl then R⁴ is not unsubstituted 2-naphthyl.

97. The compound according to claim 96, wherein at least one of R^{N5} and R^{N6} is H, and the other is selected from H and C(═O)Me.

98. The compound according to claim 96, wherein R⁵ is H.

99. The compound according to claim 96, wherein R⁴ is an optionally substituted 3- or 4-C₅₋₆ aryl-C₅₋₆ aryl group.

100. A compound of formula IIIa or IIIb: 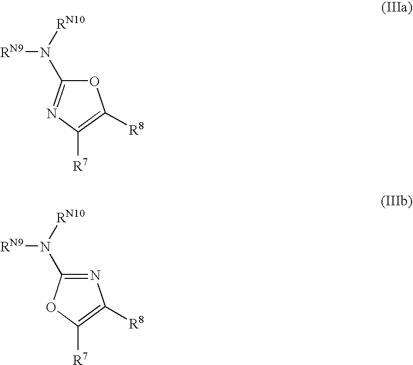
or a salt, solvate and chemically protected form thereof,
wherein:
R⁸ is selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl;
R⁷ is an optionally substituted bi-C₅₋₇ aryl group;
R^{N9} and R^{N10} are either:
(i) independently selected from H, R, R′, SO₂R, C(═O)R, (CH₂)ₙNR^{N11}R^{N12}, where n is from 1 to 4 and R^{N11} and R^{N12} are independently selected from H and R, where R is optionally substituted C₁₋₄ alkyl, and R′ is optionally substituted phenyl-C₁₋₄ alkyl, or
(ii) together with the nitrogen atom to which they are attached, form an optionally substituted C₅₋₇ heterocyclic group;
with the proviso that in formula IIIb, when R^{N9}, R^{N10} and R⁸ are H, R⁷ is not 4-phenyl-phenyl.

101. The compound according to claim 100, wherein R⁸ is selected from H and optionally substituted C₁₋₆ alkyl.

102. The compound according to claim 100, wherein R^{N9} and R^{N10} are independently selected from H and R.

103. The compound according to claim 102, wherein R⁷ is an optionally substituted bi-phenyl group.

104. A compound of formula IVa or IVb: 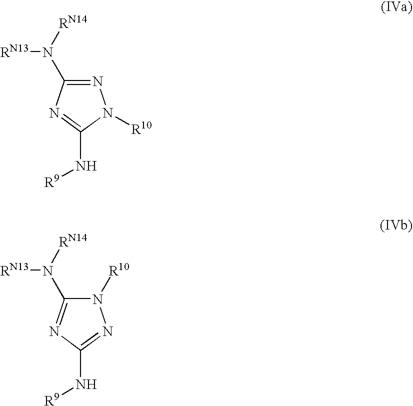
or a salt, solvate and chemically protected form thereof, wherein:
R¹⁰ is selected from the group consisting of H and optionally substituted C₁₋₆ alkyl;
R⁹ is an optionally substituted C₉₋₁₄ aryl group or an optionally substituted bi-C₅₋₇ aryl group;
R^{N13} and R^{N14} are either:
(i) independently selected from H, R, R′, SO₂R, C(═O)R, (CH₂)ₙNR^{N15}R^{N16}, where n is from 1 to 4 and R^{N15} and R^{N16} are independently selected from H and R, where R is optionally substituted C₁₋₄ alkyl, and R′ is optionally substituted phenyl-C₁₋₄ alkyl, or
(ii) together with the nitrogen atom to which they are attached, form an optionally substituted C₅₋₇ heterocyclic group,
with the proviso that when R¹⁰, R^{N13} and R^{N14} are H, R⁹ is not an unsubstituted naphthyl group.

105. The compound according to claim 104, wherein R¹⁰ is selected from H and optionally substituted C₁₋₆ alkyl.

106. The compound according to claim 104, wherein R^{N13} and R^{N14} are independently selected from H and R.

107. The compound according to claim 104, wherein R⁹ is an optionally substituted bi-phenyl group.

108. A method of treating a condition which can be alleviated by antagonism of a 5-HT_{2B} receptor, which method comprises administering to a patient in need of treatment an effective amount of a compound according to claim 92.

109. A method of treating a condition which can be alleviated by antagonism of a 5-HT_{2B} receptor, which method comprises administering to a patient in need of treatment an effective amount of a compound according to claim 96.

110. A method of treating a condition which can be alleviated by antagonism of a 5-HT_{2B} receptor, which method comprises administering to a patient in need of treatment an effective amount of a compound according to claim 100.

111. A method of treating a condition which can be alleviated by antagonism of a 5-HT_{2B} receptor, which method comprises administering to a patient in need of treatment an effective amount of a compound according to claim 104.
